(19) United States
(12) Patent Application Publication
(10) Pub. No.: US 2026/0325004 A1
(43) Pub. Date: Oct. 22, 2026

(54) APPARATUS FOR IN SITU MEASUREMENT OF ELECTRICAL IMPEDANCE OF LUNG TISSUE

(71) Applicants: The Trustees of the Stevens Institute of Technology, Hoboken, NJ (US); The Trustees of Columbia University in the City of New York, New York, NY (US); Vanderbilt University, Nashville, TN (US)

(72) Inventors: Jinho Kim, Jersey City, NJ (US); Gordana Vunjak-Novakovic, New York, NY (US); Meghan Pinezich, New York, NY (US); Seyed Mohammad Mir, Hoboken, NJ (US); Jiawen Chen, Hoboken, NJ (US); Maria Hudock, New York, NY (US); Matthew Bacchetta, Nashville, TN (US)

(73) Assignees: The Trustees of the Stevens Institute of Technology, New York, NY (US); The Trustees of Columbia University in the City of New York, New York, NY (US); Vanderbilt University, Nashville, TN (US)

(21) Appl. No.: 19/182,362

(22) Filed: Apr. 17, 2025

(51) Int. Cl. A61B 5/085; A61B 5/00; A61B 5/0538

(52) U.S. Cl. CPC A61B 5/085; A61B 5/0538; A61B 5/6852; A61B 5/0048

(57) ABSTRACT

Apparatus and methods are disclosed to assess tissue properties, especially those of lung tissues. By measuring impedance in response to electrical stimulation, a variety of properties can be deduced including spatial location of damaged tissue, presence of epithelial cells and function of diseased tissue. To these ends, a probe (10) with electrodes (12, 14, 16, 18) connected to an impedance analyzer (20) was developed for assessing tissue (22) function. By applying voltages and/or currents, different information can be determined. In another embodiment, such a probe can be used for electroporation to evaluate the location of genetic molecules (e.g., RNA, DNA, or gene-editing machinery) and/or encourage their transport across different layers of tissue.

### CROSS-REFERENCE TO RELATED APPLICATION

This application is a continuation in-part of International Application No. PCT/US2023/077227 filed Oct. 18, 2023, which claims priority to U.S. Provisional Patent Application Ser. No. 63/417,641 filed Oct. 19, 2022, the entire disclosures of both of which being incorporated herein by reference.

### STATEMENT REGARDING FEDERALLY SPONSORED RESEARCH

This invention was made with government support under Grant No. 2143620 awarded by the National Science Foundation and Grant No. EB027062 awarded by the National Institutes of Health. The government has certain rights in the invention.

### FIELD OF THE INVENTION

The invention relates to medical instrumentation for taking measurements, and specifically, devices and systems that can allow real-time in situ detection of mucus layer, exogenous gene distribution, biosensing of different molecules and proteins, detection of abnormal tissues such as tumors and fibrotic tissues and diagnosis of respiratory diseases, such as chronic obstructive pulmonary diseases (COPDs), cystic fibrosis (CF), asthma, primary ciliary dyskinesia (PCD), acute lung injury, pulmonary edema, and pulmonary fibrosis.

### BACKGROUND OF THE INVENTION

The human conducting airways are lined by the airway epithelium that consists of multi-ciliated, club, goblet, and basal cells. The airway epithelial layer creates a protective biophysical barrier between the external environment and underlying tissues against inhaled harmful substances, such as pathogens, allergens, chemical gases, or particulates. The protective functions of the airway epithelium include mucociliary clearance, tight junction formation, and antimicrobial secretion. Unfortunately, the airway epithelium is repeatedly injured or diseased, and thus is frequently the prime site for the initiation and progression of many devastating respiratory disorders, such as cystic fibrosis (CF), asthma, chronic obstructive pulmonary diseases (COPD), or primary ciliary dyskinesia (PCD).

The airway epithelial cells lining the luminal surface of the respiratory tract collectively generate protective barrier functions, including mucociliary clearance, intercellular junction formation, and antimicrobial secretion. In particular, the mucociliary flow is a unidirectional surface flow that moves across the airway lumen to ensure clearance of the inhaled materials″. This unidirectional flow is produced in the direction from the distal (D) to proximal (P) lung regions by coordinated, repeated, and polarized beating of cilia of multiciliated cells (FIG. 12A). Polarized arrangement of these cells along the P-D axis, which is known as planar cell polarity (PCP), ensures generation of the unidirectional flow. Tissue-level PCP is established and maintained by Frizzled (Fzd)/Dishevelled (Dvl) and Van Gogh-like (Vangl)/Prickle (Pk) protein complexes that are arranged at the proximal and distal cell membrane domains, respectively (FIG. 12B). Further, tight and adherens junctions formed between epithelial cells play an important role in generation of the defensive epithelial barrier. Transmembrane proteins, such as Zonula occudens-1 (ZO-1) and E-cadherin, promote tight connection between the membranes of adjacent epithelial cells, regulating paracellular permeability.

As the first defensive mechanism of the lung, the epithelial barrier is frequently challenged and injured by inhaled harmful particles, gases, or pathogens, or through physical trauma that occurs during clinical procedures, such as intubation and anastomosis (FIG. 12C). While minor injury can be resolved quickly by intrinsic repair mechanisms of the healthy airway tissue, severely or repeatedly injured epithelium can cause prolonged or permanent failure in repair or reduced level of restoration relative to normal tissue integrity and functions. Disruption in the tissue repair and regeneration pathways and loss of the epithelial cells' ability to restore the defensive barrier could result in aberrant remodeling and structural damage that can further impair epithelial functions. Acute or persistent structural damage induced to the airway tissue is known to degrade the epithelial barrier functions. However, the correlation between the structure and function of the airway epithelium remains largely elusive.

The inhalation of harmful substances can cause substantial damage to the pulmonary epithelium, potentially leading to lung dysfunction. For instance, the aspiration of large amounts of gastric acid triggers inflammation in the lung epithelial cells, ultimately resulting in acute respiratory distress syndrome (ARDS) and pneumonia. ARDS is characterized by severe airway and lung parenchyma injury, with a high mortality rate (40-70%) even with access to intensive care. Therefore, early and precise diagnosis of acid-induced injury, as a risk factor for the development of ARDS, is critical to provide effective treatments.

Different types of bioimpedance measurement techniques have been developed and used extensively to evaluate the structure, content, and function of biological tissues and organs. For example, electrical impedance tomography (EIT) is an imaging modality that can visualize internal organs, such as lung, by mapping electrical properties of the patient's body in a non-invasive and radiation-free manner. Similarly, the percentages of body fat and muscle mass can be estimated indirectly by measuring the bioimpedance across a human body.

Rapid and accurate monitoring and quantification of airway epithelium injury or disease can maximize treatment outcomes by allowing expedited establishment of appropriate treatment modalities. However, there has been a dearth of ways to determine the extent of airway epithelium injury or disease within the respiratory tract of the lung.

In injured and diseased tissues, changes in molecular and cellular compositions, as well as tissue architecture, can lead to alterations in both physiological and physical characteristics. Notably, the electrical properties of tissues, which can be characterized as bioelectrical impedance (bioimpedance), are closely linked to the health and pathological conditions of the tissues. This highlights the role of quantitatively characterizing these electrical properties by improving the accuracy and speed of diagnosis and prognosis.

The lungs are vital organs for gas exchange. However, they are constantly exposed to foreign materials, such as pathogens, allergens, dusts, and pollutants [1,2]. A combination of environmental insults and genetic factors gradually leads to the development and progression of pulmonary acute and chronic conditions. These pathological conditions are associated with deregulation of cells and extracellular matrix (ECM) composition and structure. For example, the airway epithelial cells lining the luminal surface of the respiratory tract are repeatedly injured by inhaled toxic material, leading to chronic obstructive pulmonary diseases (COPDs). Moreover, environmental factors have been linked to damage of alveolar type 2 (AT2) cells and contribute to the development of idiopathic pulmonary fibrosis a progressive diseased marked by chronic inflammation and progressive scarring of the lungs. Different lung injuries, such as injury caused by gastric acid aspiration and chemicals, may eventually lead to acute respiratory distress syndrome (ARDS), a life-threatening condition characterized by accumulation of protein-rich fluids in the alveoli (i.e., pulmonary edema) and disruption of the surfactant layer. In lung cancer, the leading cause of cancer-related deaths worldwide, the ECM microenvironment deposited by tumor cells undergoes significant changes during the remodeling process. Many diagnostic technologies, such as advanced imaging and diagnostic tools, have been developed for lung diseases. However, many of these diseases are often difficult to detect, especially in their early stages, due to their complex pathophysiology. As a result, they are frequently diagnosed only in advanced stages. As such, alternative methods to characterize tissues affected by diseases and injuries to improve diagnostic capabilities and enhance treatment outcomes are desirable.

Earlier studies suggest a strong correlation between the pathological state of tissues, which affects the biological composition and structure of the tissues, and their electrical properties, particularly bioelectrical impedance (i.e., bioimpedance). For example, cancerous tissues typically contain higher water and sodium concentrations and cell necrosis compared to normal tissues, which collectively leads to lower resistance to electrical current (i.e., low bioimpedance). Moreover, changes in bioimpedance have been observed with the progression of fibrosis in tissues. Notably, the bioimpedance of tissues and cells has shown frequency-dependent behavior in response to the applied electrical signal (e.g., alternating current, AC). The cell membrane is highly resistive to low-frequency electrical current; therefore, tissue bio-impedance at low frequencies primarily reflects the extracellular environment. At high frequencies, the electrical signal can penetrate the cell membrane, whereby the bioimpedance is attributable to both intra and extracellular environments. Therefore, measuring bioimpedance across a frequency range can provide valuable information about characteristics of the underlying ECM components, the cell membrane, the intra and extracellular fluids, and the pathological state of tissue.

Tissue bioimpedance is typically measured by injecting an alternating current at different frequencies between two electrode probes to a region of interest and measuring the resulting voltage between the electrodes through which the current is injected. Existing bioimpedance measurement techniques can offer noninvasive, label-free, and real-time assessments to characterize biological tissues and cells. For example, bioelectrical impedance analysis (BIA), also known as electrical impedance spectroscopy (EIS), has been used to estimate body composition, such as the amount of adipose tissue, muscle, bone, and level of fluid in the body. Such data are used by physicians for prognostication, personalized nutrition, fluid therapy, and medication management when caring for their patients. At the cellular level, bioimpedance measurement has been used to monitor the proliferation and differentiation of various cells, such as mesenchymal stem cells, cardiac cells, neural stem cells, as well as monitoring for degree of toxicity and inflammation, real-time attachment and spread of cells onto substrates, cell viability, and cell migration.

Electrical impedance tomography (EIT) has been used as a noninvasive approach to image organs, such as the lung, by measuring their electrical properties externally. An EIT system consists of an array of electrodes, a current-generating device, a multichannel voltage detection system, and an image construction algorithm to convert the measured data into a two-dimensional (2D) tomogram. EIT has been used to measure the bioimpedance of lung regions to assess pulmonary functions. Specifically, EIT provides valuable information regarding lung aeration and volumes during ventilation. Notably, this approach can give insights into detecting recruitment, decruitment, and overdistension of lung regions in response to positive end-expiratory pressure titration strategies, atelectasis, intratidal regional gas distribution on a breath-by-breath basis, and measurement of regional time constants in patients with ARDS. Moreover, several studies have explored the application of EIT in the detection of lung pathologies, such as COPD, cystic fibrosis (CF), asthma, and pulmonary hypertension (PH), and pleural effusion.

### SUMMARY OF THE INVENTION

The present invention relates to a deployable electrode assembly that can be inserted into the respiratory tract during video bronchoscopy for real-time in situ measurements of electrical impedance of the pulmonary liquid lining the airway lumen, such as mucus, and the airway tissue layers, such as epithelium and extracellular matrix (ECM). Measured electrical impedance at local airway regions can be used to diagnose different airway diseases, such as CF, asthma, COPD, and PCD, and monitor disease progression or resolution. The invention addresses the lack of commercial devices and systems that can allow real-time in situ detection of mucus layer, exogenous gene distribution, biosensing of different molecules and proteins, and diagnosis of diseases within the respiratory tract.

For instance, electrical impedance tomography (EIT) is an imaging modality that can allow non-invasive monitoring of internal organs and tissues by quantifying electrical impedance, conductivity, and permittivity of the target tissues and organs. However, EIT is used to visualize internal organs, such as the lungs, based on their electrical properties measured externally. Unlike EIT imaging, the present invention uses electrode probe(s) that can be inserted into the respiratory tract or any other body parts for direct, real-time, and accurate measurements of the electrical properties of local tissue that can be correlated to the injury or disease states of the tissue. The present invention mainly focuses on electrical assessment of airway tissues. It can also be used to evaluate tissues of any other internal organs, such as the liver, gallbladder, or kidney; or hollow tissues or organs, such as the intestines, blood vessels, esophagus, fallopian tubes, stomach, bladder, or inner ear.

The present invention relates to an apparatus that can allow for real-time in situ assessment of the electrical impedance of liquid lining the airway lumen or different tissue layers within the respiratory tract. The apparatus can include a deployable electrode array that can be introduced into the respiratory tract via a video bronchoscope, directly contact the surface of the airway inner lumen in selected regions of the lung and determine the electrical impedance of the pulmonary liquid or tissue layers (FIG. 1).

The underlying connection between tissue electrical properties and lung diseases underscores the value of accurate quantitative assessment of electrical characteristics of lung tissues, which can enhance understanding of diseases for early diagnosis to guide effective treatment. The current literature mostly investigates the bio-impedance of the lungs primarily through placing the electrodes around the thoracic region (e.g., EIT). However, few studies have specifically focused on bioimpedance measurements through direct contact of electrode probes with the lung tissues. This approach can have significant potential for tissue quality evaluation in operating rooms, in situ evaluation of lungs supported in the bioreactor, and development of lung therapeutics. For example, collecting bio-impedance information from direct contact with local tissue regions contact allows surgeons to identify changes in airway tissues during bronchoscopy examination or to obtain regional insight and information about tissue quality during open and laparoscopic lung surgeries. Moreover, bioimpedance measurement can serve as an evaluation tool for the trachea and lungs cultured in bioreactors during different tissue manipulations, such as decellularization and recellularization. Therefore, the present invention aids the evaluation of bioimpedance of ex vivo lung tissues, including trachea and lung parenchyma, by placing the electrode probes onto the surfaces of these tissues (FIGS. 28A-28C). Lungs were explanted from Sprague-Dawley rats and measurements were conducted ex vivo. FIG. 28A is a schematic showing the linear arrangement of electrode probes on lung and trachea tissues. FIG. 28B is a schematic of the system to measure bioimpedance. The probes were attached to XYZ manipulators for precise manipulation. A custom-built camera monitored the arrangement of the probes on the tissue surface. FIGS. 28Ci and 28Cii are schematics showing the side-view images of the probes positions on (i) lung pleura and (ii) trachea tissue surfaces. I: Current. V: Voltage. CC: Current-carrying electrode probe. VS: Voltage-sensing electrode probe.

Specifically, the bioimpedance was characterized under different experimental conditions and in various tissue states. The results indicated that tissue bioimpedance heavily depends on lung tissue ultrastructure, composition, and pathological state. The findings of the study can expand knowledge regarding the correlations between lung pathologies and bioimpedance and increase the diagnostic capabilities for detecting lung tissue impacted by injuries and diseases.

In one embodiment of the present method, electrical properties of a trachea can be measured using an LCR meter (e.g., E4980AL, Keysight). Specifically, electrical resistance (R) and reactance (X) of the tissues can be measured at various alternating current (AC) frequencies, ranging between 20 Hz and 135 kHz. Bioimpedance (Z) of the tissues can be calculated using the acquired R and X values via Z=R+jX. The four-probe measurement technique is beneficial by improving the accuracy of the measurements. For instance, four tungsten electrodes (e.g., SE-T, Lucas Signatone; length: 30 cm; tip diameter: 5 μm) may be used. To place the electrodes at precise locations during measurements, each electrode may be mounted on a micropositioner (e.g. m S-725SLM or S-725SRM, Lucas Signatone; see FIG. 14A). The position of the electrode tip can be monitored using a custom-built microscope, integrated with a 10×LWD objective lens (375-039, Mitutoyo), a tube lens (AC254-100-A-ML, Thorlabs), and a scientific CMOS camera (pco.panda 4.2, PCO). It was confirmed that epithelial injury can be diagnosed in situ by measuring electrical properties of the local airway tissue directly at the injury site.

The Cole model may be used to analyze the measurement results and provides the bioimpedance as:

$$Z ( s ) = R + jX = R ∞ + \frac{{R}_{0} - {R}_{∞}}{1 + {( j ω τ )}^{a}}$$

where R_{∞} is the resistance at infinite AC frequency, R₀ is the resistance at zero AC frequency (i.e., the direct current, DC), ω is the angular frequency, τ is the time constant of the dispersion, and α is an empirical exponent. Through nonlinear curve fitting, the data may be fitted to a semi-circle curve with its center (x₀, y₀) and radius (r₀). To minimize the curve fitting error, the least absolute deviation (LAD) method may be used. One effective fit was determined to be the one that generated the minimal value of the function:

$${f ( {x}_{0} , {y}_{0} , {r}_{0} ) = {\sum }_{i = 1}^{n} \vert \sqrt{( {x}_{i}} - {x}_{0} )}^{2} + {( {y}_{i} - {y}_{0} )}^{2} - {r}_{0} \vert$$

where n is the number of data points. The parameters of the Cole model can then be extracted from the curve. For example, the two points where the curve intersects with the horizontal axis (i.e., resistance axis) represent R₀ and R_{∞}, respectively. α is an empirical exponent that serves as a measure of the position of the circle center below the horizontal axis and can be calculated according to the relationship between α and θ:

$$θ = \frac{π α}{2 {r}_{0}}$$

where θ can be calculated from the geometrical relationship: sin

$$θ = \frac{{R}_{0} - {R}_{∞}}{2 {r}_{0}}$$

τ corresponds to the inverse of the angular frequency at the highest reactance:

$$τ = \frac{1}{2 π {f}_{c}}$$

where f_{c} is the frequency at the highest reactance.

To rationalize the acquired experimental data and identified epithelial structure-function relationships, computational models that were created can numerically simulate and predict the impairment of the epithelial barrier functions. These findings can contribute to development of innovative opto-electrically enabled diagnostic modalities coupled with computational models that can accurately quantify and predict initiation, progression, and resolution of epithelial injury.

For instance, the electric current physics module of COMSOL was used to simulate the bioimpedance of both native and injured airway tissues with varied AC frequencies between 10 Hz and 135 kHz. The tissue model (length: 6 mm, width: 4 mm, and height: 545 μm) can consist of four distinct layers that represent the pulmonary surface liquid (thickness: 15 μm), epithelium (thickness: 30 μm), submucosa (thickness: 200 μm), and cartilage (thickness: 300 μm). In the tissue model with epithelium injury, a small cylindrical volume (diameter: 0.5 mm; height: 29 μm) was incorporated into the epithelium that represents removed tissue from the mucosa. Four circles (diameter: 0.5 μm), each of which represents the tissue regions that are in contact with the electrode, were created near the injury site. The electrical properties of the fluid and tissue layers were obtained from the literature. Fine mesh was created for the simulation and the solution was obtained in frequency domain. Simulation results, such as impedance and volume impedance density, were calculated using existing methods. Briefly, sensitivity (S) was calculated to indicate how much the resistivity of a particular point influences the total impedance according to the following equation for a four-electrode system:

$$S = \frac{\vec{JCC} \cdot \vec{JPU}}{{I}_{cc} {I}_{PU}}$$

where \vec{J_{cc}} and \vec{J_{PU}} are the current density of the current carrying (CC) electrodes and potential pick-up (PU) electrodes, respectively. I_{CC} is the current in the CC electrodes and I_{PU} is the reciprocal current in the PU electrodes. The volume impedance density (VID) can be obtained by multiplying the sensitivity with the complex resistivity (ρ) as shown in the following equation: VID=Sρ to find the contribution from each constituent of the model to the total impedance measured in this model. If the volume impedance density is integrated over all points in the measurand, the result is that the measured impedance Z=∫∫∫JVID dv.

In one aspect, the invention comprises a deployable electrode assembly for real-time in-situ airway impedance measurements whose distal ends are exposed with electrodes that can directly touch the airway luminal surface while its proximal end is connected to an impedance analyzer (FIGS. 1-5). In another embodiment, the present invention pertains to a method of use of this apparatus to determine presence and removal of airway mucus lining the airway lumen (FIGS. 6-7). Another aspect of the present invention is a method of use of this apparatus to determine the spatial distribution of genes across the airway tissue following the delivery of genes, gene delivery vehicles, or gene-editing machinery (FIG. 8). A further aspect of the present invention involves a method of use of this apparatus to maximize internalization and endosomal escape of genes, gene delivery vehicles, or gene-editing machinery into the airway epithelial cells via a closed-loop in situ electroporation and impedance measurement (FIG. 9). In an additional aspect of the present invention, it comprises a method of use of this apparatus to apply electrode biosensors to monitor airway disease in situ through biosensing of bacteria, fungi, virus, microbial biofilms, other pathogens, cell-free DNA, airway edema, matrix protein fragments, and infiltrating immune cells (FIG. 10). Another aspect of the present invention is a method of use of this apparatus to evaluate the quality of donor organs via impedance-based detection of pathologic tissues (FIG. 11). In a not-necessarily-final aspect of the present invention, the inventive device can be coupled with existing endoscopic devices or robotic surgical devices to measure electrical impedance of local tissues.

Measuring the electrical impedance of lung tissue has great potential to be a non-invasive and accurate diagnostic tool for certain lung diseases and injuries. In the clinical setting, electrical impedance tomography (EIT) is frequently used to continuously monitor lung ventilation and perfusion, alveoli recruitment or derecruitment, and alveoli overdistension. The present invention suggests that electrical bioimpedance can be used as an efficient and accurate diagnosis tool for acute acid-induced lung injury (FIGS. 22A-22C and 27A-27I) and pulmonary fibrosis (FIGS. 23A-23D).

In an embodiment, the present invention relates to a method of assessing tissue function or conditions by placing a probe in contact with a tissue of interest, the probe having at least one electrode configured to deliver electrical current to the tissue of interest and at least another electrode configured to take potential measurements of the tissue of interest, delivering electrical current to the tissue of interest via the at least one electrode of the probe, taking potential measurements of the tissue of interest using the at least another electrode of the probe, calculating impedance measurements in response to the potential measurements, and assessing tissue function or conditions based on said impedance measurements. The electrodes may be made of tungsten. The electrodes can be finely positioned on the tissue of interest using a micropositioner device. In this vein, the position of the electrodes relative to the tissue of interest can be aided using a video bronchoscopy device.

In another embodiment of the present invention the assessment of tissue function can involve of monitoring the spatial arrangement of genes or gene-editing machinery in proximity to the tissue of interest. For instance, the delivering of electrical current can involve delivering high frequency current to determine the spatial arrangement of genes and/or gene-editing machinery internalized within cells and/or delivering low frequency current to determine the spatial arrangement of genes and/or gene-editing machinery external to cells. In a similar vein, in lung tissue applications, genes or gene editing machinery can be deposited on the luminal surface of the lung tissue, and electroporation the luminal surface to internalize said genes or gene-editing machinery into cells can follow.

The method tissue of interest is a donor organ. In such applications, the impedance measurements can localize injured or diseased tissue or defects in the tissue of interest. When the tissue of interest is lung tissue, the assessment of a mucus layer in the respiratory tract can be performed. Generally speaking, the present invention can be used to diagnose one or more airway diseases using the impedance measurements.

In yet another embodiment of the present invention, the electrodes can have functionalized surfaces. In these instances, the electrodes can recognize and bind their functionalized surfaces to specific target molecules, proteins, or pathogens. Then the change in electrical properties of the electrode can be measured after such recognizing and binding.

The delivery of electrical current and subsequent measuring can be performed via a frequency sweep of said electrical current. For example, the following frequency ranges can be used for such a frequency sweep: 100 Hz to 300 kHz; 100 Hz to 135 kHz; and 30 Hz to 300 kHz. Alternatively, the electrical current can be direct current.

The measurement parameters may be varied for variable penetration of electrical current into the tissue of interest, effectively varying the depth of penetration of said electrical current and allowing for impedance measurements at different depths. Such parameters can include frequency of the electrical current, spatial arrangement of the electrodes, and size the electrodes.

Another embodiment of the present invention relates to apparatus for evaluating the function or conditions of a tissue of interest that includes a probe with a plurality of electrodes adapted to be placed in direct contact with a tissue of interest, including at least one electrode adapted to deliver electrical current to the tissue of interest and at least another electrode adapted to make potential measurements of the tissue of interest, an impedance analyzer adapted to receive the potential measurements from the probe, wherein the impedance analyzer is adapted to evaluate tissue properties or conditions based on the potential measurements. The apparatus can further include a micropositioner adapted to place the electrodes on the tissue of interest.

In certain applications, the apparatus can be integrated in a video bronchoscopy device or a robotic surgery device. The impedance analyzer can also adapted to determine the presence of defects or diseased tissue in a donor organ. The impedance analyzer can also be adapted to determine the presence or condition of a mucus layer in the respiratory tract. The electrodes may also have surfaces functionalized with molecules, proteins, aptamers or antibodies, such that the electrodes can recognize and bind to specific target molecules, proteins, or pathogens. For example, such specific target molecules can be pathogens associated with airway injury or disease.

Further results showed that rat lung and trachea tissues exhibit a frequency-dependent behavior to alternating current (AC) across the frequency range of 0.1-300 kHz. The bioimpedance of the lung tissue increased with the level of aeration of the lung, which was manipulated by altering alveolar pressure (P_{ALV}: 1-15 cmH2O; bioimpedance level: 1.2-2.8 kΩ; AC frequency: 2 kHz). This increase is mainly because air is electrically nonconductive. The bioimpedance of rat lungs injured via intratracheal aspiration of hydrochloric acid (HCl; volume: 1 mL; AC frequency: 2 kHz) decreased by at least 82% compared to that of healthy control lungs due to accumulation of fluids inside the airspace of the injured lungs. Moreover, using decellularized lung tissues, the contributions of cellular components and tissue extracellular matrix (ECM) on the electrical characteristics of the lung tissues was determined. Specifically, a considerable increase in bioimpedance in fibrotic human lung tissues due to excessive ECM deposition (healthy: 70.8 Ω±10.2Ω, fibrotic: 132.1 Ω±15.8Ω, frequency: 2 kHz) was observed. Overall, the findings enhance understanding of the correlation between electrical properties and pathological lung conditions, thereby improving diagnostic and prognostic capabilities and aiding in the treatment of lung diseases and injuries.

### BRIEF DESCRIPTION OF THE DRAWINGS

For a more complete understanding of the present invention, reference is made to the following detailed description of various embodiments considered in conjunction with the accompanying drawings, in which:

FIG. 1A is a schematic showing a deployable device that can measure the electrical impedance within a local airway in accordance with an embodiment of the present invention, showing the airway impedance measurement device coupled with a bronchoscope and inserted into the respiratory tract.

FIG. 1B is a schematic showing a deployable device that can measure the electrical impedance within a local airway in accordance with an embodiment of the present invention, showing the measurement head of the device contacting the surface of the airway inner lumen to quantify the impedance of local tissue, the electrode array being exposed at the distal end of the device that can directly contact the airway surface during impedance measurement.

FIGS. 2A-2C are a series of photographs showing a prototype of an airway impedance measurement device, showing an impedance measurement system that includes a custom-built electrode assembly (FIG. 2A), a custom-built electrode assembly (FIG. 2B), and a hydrogel-based tissue phantom whose electrical impedance is being measured using the system (FIG. 2C);

FIG. 3 is a series of photographs, showing the impedance measurement system, the custom-built electrical probe, and hydrogel tissue phantoms being tested for impedance measurements, wherein FIG. 3A is a schematic diagram illustrating in situ measurement of electrical impedance across the airway tissue to quantify the mucus layer, in accordance with an embodiment of the present invention, FIG. 3B is a photograph, showing an impedance measurement system including the impedance meter and electrode probes, FIG. 3C is a photograph showing a custom-built electrical probe and FIG. 3D is a photograph showing a hydrogel tissue phantom being tested for impedance measurement, in accordance with embodiments of the present invention;

FIG. 4A is a photograph showing a rat liver being isolated and tested for impedance measurements, in accordance with an embodiment of the present invention;

FIG. 4B is a graph showing impedance measurements of the rat liver of FIG. 4A, while varying the frequency of AC current from 30 Hz to 300 kHz in accordance with an embodiment of the present invention, demonstrating that as the frequency increased, the impedance of the tissue decreased because higher frequency current can pass through the cells more easily;

FIG. 5A is a cross-sectional view of the airway tissue through which injected electrical current is flowing, in accordance with an embodiment of the present invention;

FIG. 5B is an electric diagram illustrating an electrical circuit in accordance with an embodiment of the present invention, wherein R_{PLL} is the resistance of the pulmonary liquid layer, R_{ECF} is the resistance of the extracellular fluid, R_{ICF} is the resistance of the intracellular fluid, R_{ICF-ECM} is the resistance of the intracellular fluid within the extracellular matrix (ECM), C_{CM} is the capacitance of the epithelial cell membrane, and C_{CM-ECM} is the capacitance of the sub-epithelial cell membrane;

FIG. 5C is an equation for calculating overall impedance measured at the airway tissue, in accordance with an embodiment of the present invention, wherein R₀ is the resistance measured with zero frequency (i.e., DC) and R∞ is the resistance measured with infinite frequency AC, wherein α is a correlation factor with a value between 0 and 1 that is empirically determined, where τ corresponds to the inverse of the angular frequency at the highest reactance;

FIG. 6 is a schematic diagram showing determination of the electrical impedance at different tissue depths in accordance with an embodiment of the present invention, wherein tissue penetration of electrical current depends on measurement parameters, such as frequency of injected AC current, spatial arrangement of the electrodes, and size of the electrodes contacting the tissue, and thus, by modulating the measurement parameters, the impedance of the airway tissue at different depths could be determined, for example, by adjusting the distance between electrodes (L), thereby modulating the penetration depth of injected current (D) and allowing impedance measurements at different tissue depths;

FIG. 7A is a schematic diagram illustrating in situ measurement of electrical impedance of the airway tissue to quantify the mucus layer presence or thickness lining the airway lumen surface in accordance with an embodiment of the present invention, showing a deployable electrode assembly can be inserted into the trachea and the impedance of the airway tissue can be measured to identify the presence of the mucus or the thickness of the mucus layer. The electrode can supply electrical current while measuring the voltage to determine the impedance of the airway tissue near the luminal surface;

FIG. 7B is a schematic diagram illustrating in situ measurement of electrical impedance of the airway tissue to quantify the mucus layer presence or thickness lining the airway lumen surface in accordance with an embodiment of the present invention, showing that because a thick mucus layer will increase the impedance of the airway tissue, cleared airway lumen will be indicated by reduced impedance;

FIG. 8A is a schematic diagram depicting a determination of the location of gene (or gene editing machinery) across the airway tissue in accordance with an embodiment of the present invention, illustrating that because DNA or RNA strands used for gene editing are electrically negatively charged, their spatial distribution across the airway tissue can be indicated by alteration in the electrical impedance values measured with varied measurement conditions, such as AC current frequency and probe arrangement;

FIG. 8B is a schematic diagram depicting a determination of the location of gene (or gene editing machinery) across the airway tissue in accordance with an embodiment of the present invention, illustrating that low frequency current (<1 kHz) will tend to travel through the extracellular fluids due to high impedance imposed by the cell membranes, while higher frequency current (>100 kHz) can pass through the cell membranes. Thus, DNA or RNA remaining outside the cells could be detected by impedance measured at low current frequency and those internalized into the cells can be detected by the impedance measured at high frequency electrical current;

FIG. 9A is a schematic diagram illustrating in situ electroporation to promote internalization of genes into epithelial cells, in accordance with an embodiment of the present invention, illustrating that the electrode assembly can be used to provide a pulse of high-voltage electricity (e.g., pulse duration: 150 ms; electric field strength: 200 V/cm) across the airway tissue to facilitate internalization of the genes via electroporation into the internal space of the epithelial cells. In this approach, the genes (or gene editing machinery) can be deposited onto to the airway lumen and are exposed to the electrical pulse for the internalization;

FIG. 9B is a schematic diagram illustrating in situ electroporation to promote internalization of genes into epithelial cells, in accordance with an embodiment of the present invention, illustrating that through the impedance measurement, spatial distribution of the genes can then be estimated;

FIG. 9C is a flow chart illustrating in situ electroporation to promote internalization of genes into epithelial cells, in accordance with an embodiment of the present invention, wherein when needed, the electroporation and subsequent impedance measurement can be repeated to maximize gene internalization;

FIG. 10A is a schematic diagram illustrating in situ biosensing of disease or injury-associated molecules, proteins or pathogens in accordance with an embodiment of the present invention, wherein the electrode can be designed in a way that its surface can be functionalized with molecules or proteins, such as aptamers or antibodies, that can recognize and bind to specific target molecules, proteins, or pathogens associated with airway injury or disease;

FIG. 10B is a schematic diagram illustrating in situ biosensing of disease or injury-associate molecules, proteins or pathogens in accordance with an embodiment of the present invention, illustrating that binding of these molecules, proteins, or pathogens onto the sensor surface can result in changes in the electrical properties of the sensor that can allow diagnosis of airway tissue injury or disease;

FIG. 11 is a schematic showing impedance-based assessment of donor organs in accordance with an embodiment of the present invention, showing that impedance-based assessment of donor organs at the time of organ donation or during ex vivo organ support or reconditioning can more accurately quantify the quality of the organ by using a hand-held electrode probe or a wearable probe array. For example, locations of the pulmonary edema, fibrotic or diseased tissues, or tissue integrity in donor lungs or any other organs, such as the liver, can be determined by locally measuring the electrical impedance. In donor lungs, the airspace of the lung filled with fluid (i.e., edema) will display decreased impedance compared with air-filled regions because fluid is more electrically conductive than air. Fibrotic tissue will display increased impedance due to their dense tissue that is more resistant to the electrical current;

FIG. 12A is a schematic diagram showing the epithelial barrier of an airway established by polarized, coordinated arrangement of airway epithelium, illustrating unidirectional mucociliary flow generated by cilia beating;

FIG. 12B is a schematic diagram illustrating a polarized arrangement of the airway epithelium along the proximal and distal axis via a planar cell polarity signal pathway (PCP);

FIG. 12C is a schematic of epithelial barrier disruption caused by airway tissue injury;

FIG. 13 is a photograph illustrating induction of localized acute physical injury to a rat trachea epithelium ex vivo, specifically showing an experimental setup to generate the airway tissue injury wherein localized epithelial injury was induced by gently compressing the tissue with the tip of a thin metal probe (diameter: 500 microns);

FIG. 14A is a schematic illustrating an experimental setup used to measure bioimpedance of the airway tissue in accordance with an embodiment of the present invention;

FIG. 14B is a schematic of the cross-sectional view of a rat trachea with a four-probe bioimpedance measurement system;

FIG. 14C is an equivalent electrical circuit diagram for a rat trachea;

FIG. 14D Is a Cole-Cole plot illustrating various parameters associated with FIG. 14C;

FIG. 15A is a pair of bright field images showing the luminal surfaces of native and injured trachea tissues;

FIGS. 15B-15D are graphs illustrating resistance (15B), reactance (15C) and impedance (15D) of both native and injured trachea tissues measured with different alternating current (AC) frequency values in accordance with an embodiment of the present invention;

FIG. 15E is a complex impedance plot of native and injured tracheas in accordance with embodiments of the present invention;

FIGS. 16A-16F relate to a computer simulation of bioimpedance of injured and native airway tissues, showing a 3D computer model of injured trachea used for the computer simulation (16A), a plot showing simulated values of normalized impedance (Z) of native and injured trachea with respect to AC frequency (FIG. 16B), a simulated volume impedance density (VID) profile of injured (FIG. 16C) and native (FIG. 16D) trachea tissue layer with AC frequency of 10 Hz and 135 kHz, respectively, and graphs showing VID values determined via the simulations at 10 Hz (FIG. 16E) and 135 kHz (FIG. 16F);

FIG. 17A is a table showing Cole functions with calculated parameters of a Cole-Cole bioimpedance model in accordance with an embodiment of the present invention;

FIG. 17B is a cross-sectional schematic of simulated trachea geometry;

FIG. 18A is an electric potential profile of a trachea;

FIG. 18B is a graph showing quantified potential difference (ΔV) between two voltage pick-up electrodes from simulation in accordance with an embodiment of the present invention;

FIG. 19 is a profile of an electrical field (E) generated by simulation in accordance with an embodiment of the present invention;

FIG. 20A is a cross-section profile of current density (J) obtained via simulation of both native and injured trachea at frequency of 10 Hz and 135 kHz in accordance with an embodiment of the present invention;

FIG. 20B is a graph showing quantified current density along the middle lines of FIG. 20A in both native and injured trachea at frequencies of 10 Hz and 135 kHz;

FIG. 20C is a top view of simulated current density in accordance with the simulation of FIG. 20A;

FIG. 21A is a graph illustrating a pressure-volume (P-V) loop relationship, in accordance with an embodiment of the present invention, wherein impedance was measured at three different lung airway pressures (i. 1 cmH2O; ii. 5 cmH2O, and iii. 15 cmH2O);

FIG. 21B is a series of photographs of rat lungs being tested for impedance measurement at different levels of lung airway pressure, in accordance with an embodiment of the present invention;

FIG. 22A is a photograph showing acid-injured ex vivo rat lungs, in accordance with an embodiment of the present invention;

FIG. 22B is a photograph of injured lungs in accordance with an embodiment of the present invention;

FIG. 22C is a plot illustrating impedance measurements taken in select regions using an AC current of 2000 Hz in accordance with an embodiment of the present invention;

FIGS. 23A-23B illustrate electrical impedance measurements of healthy and fibrotic human lung tissue;

FIGS. 23C-23D illustrate mechanical stiffness measurements of healthy and fibrotic human lung tissue;

FIGS. 24A-24D illustrate electrical impedance measurements of an ex vivo rat trachea;

FIGS. 25A-25D illustrate impedance measurements of native and decellularized ex vivo rat lung tissues;

FIGS. 26A-26D illustrate electrical impedance measurements of native and decellularized ex vivo rat trachea tissues in accordance with an embodiment of the present invention;

FIG. 27A is a schematic of the lung with a four-probe bioimpedance measurement system, wherein I: Current source, V: voltage meter, CC: current-carrying electrode, PU: pickup electrode;

FIG. 27B is a photograph of rat lung with measurement electrodes;

FIG. 27C is a plot of impedance (Z) versus frequency for lungs with different airway pressures (peak inspiratory pressures (PIP)) in accordance with an embodiment of the present invention;

FIG. 27D is a Cole-Cole plot (reactance (X) versus Resistance (R)) for lungs with different PIP;

FIG. 27E is a schematic showing intratracheal instillation of HCL (concentration: 0.1N) into the lungs;

FIG. 27F are photographs of healthy and injured lungs with different levels of injury;

FIG. 27G is an impedance versus frequency plot for healthy and injured lungs;

FIG. 27H is a Cole-Cole plot for healthy and injured lungs;

FIG. 27I is a pair of histological images of health and acid-injured lungs;

FIGS. 28A-28C are a schematic overview of bioimpedance measurement of ex vivo lung tissues, including lung parenchyma and trachea;

FIGS. 29A-29H illustrate the effect of electrode probes' spacing on bioimpedance measurement of ex vivo rat lung;

FIG. 30 shows bioimpedance measurement of healthy and fibrotic human lung tissue, wherein bioimpedance-elastic modulus correlation for healthy and fibrotic lungs is shown at frequency of 2 kHz;

FIGS. 31A-31C is a series of schematics showing RC circuit measurement using a Keysight E4980AL-032 LCR meter, showing a resistor (R) circuit (31A), a parallel RC circuit (31B) and measured and theoretical impedance (31C), wherein f: frequency; R: resistance; C: capacitance;

FIGS. 32A-32B are schematics of a setup for bioimpedance measurement of ex vivo lung tissues;

FIGS. 33A-33E show the effect of electrode probes' spacing on the bioimpedance of lung parenchyma tissue;

FIGS. 34A-34I illustrate the effect of electrode probes' spacing on bioimpedance measurement of gelatin-based hydrogels with different thickness levels;

FIGS. 35A-35G illustrate the effect of lung internal pressure on bioimpedance of lung parenchyma tissue;

FIG. 36. illustrates phase angles of lung parenchyma tissue at different P_{ALV} levels across the frequency spectrum (range: 100 Hz 300 kHz); φ: phase angle;

FIGS. 37A-37E are histological assessments of acid-injured ex vivo rat lung;

FIGS. 38A-38B show bioimpedance assessments of acid-injured ex vivo rat lungs, wherein measured phase angles (φ) for lung tissue treated with different HCl volumes across the frequency range of 0.1 kHz-300 kHz (38A) and Cole-Cole equation and parameter values calculated for native and acid-injured lungs (38B) can be seen;

FIGS. 39A-39B show bioimpedance measurement of ex vivo rat trachea tissue, wherein resistance (39A) and reactance (39B), and phase angles (39C) of the rat trachea at anterior (cartilage-rich) and posterior (muscle-rich) sides across the frequency range between 0.1 kHz and 300 kHz can be seen;

FIGS. 40A-40B show results for a computer-simulated electrical current flow in tracheal tissue;

FIGS. 41A-41C show bioimpedance measurements of native and decellularized rat lung tissue sections, wherein resistance (41A), reactance (41B), and phase angle (41C) are shown for native and decellularized rat lung sections (thickness: ~3 mm) across the frequency range between 0.1 kHz and 300 kHz;

FIGS. 42A-42B are H&E images of decellularized tracheas;

FIGS. 43A-43F are bioimpedance measurements of native and decellularized rat trachea, showing resistance-frequency plots for anterior (43A) and posterior (43B) sides of native and decellularized trachea tissue, reactance-frequency plots for anterior (43C) and posterior (43D) sides of native and decellularized trachea tissues, and phase angle-frequency plots for anterior (43E) and posterior (43F) sides of native and decellularized trachea tissues;

FIGS. 44A-44D show the role of ECM in bioimpedance of lung tissues;

FIGS. 45A-45B show the effect of sample thickness on four-probe impedance measurements, wherein schematics of current flow in two-dimensional (2D; 45A) and three-dimensional (3D; 45B) samples are shown; and

FIG. 46 is a table showing electrical resistance, reactance, and bioimpedance of lung tissues reported in the literature.

### DETAILED DESCRIPTION OF THE EXEMPLARY EMBODIMENT

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto.

Further, it should be noted that, as recited herein, the singular forms “a”, “an”, “the”, and “one” include the plural referents unless otherwise stated. Additionally, the terms “comprises” and “comprising” when used herein specify that certain features are present in that embodiment, however, this phrase should not be interpreted to preclude the presence or addition of additional steps, operations, features, components, and/or groups thereof.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

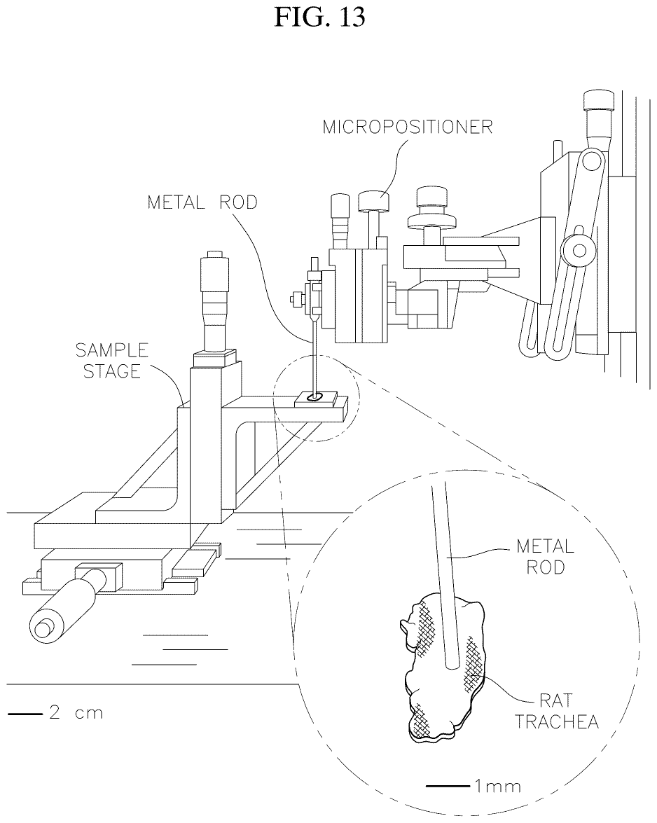

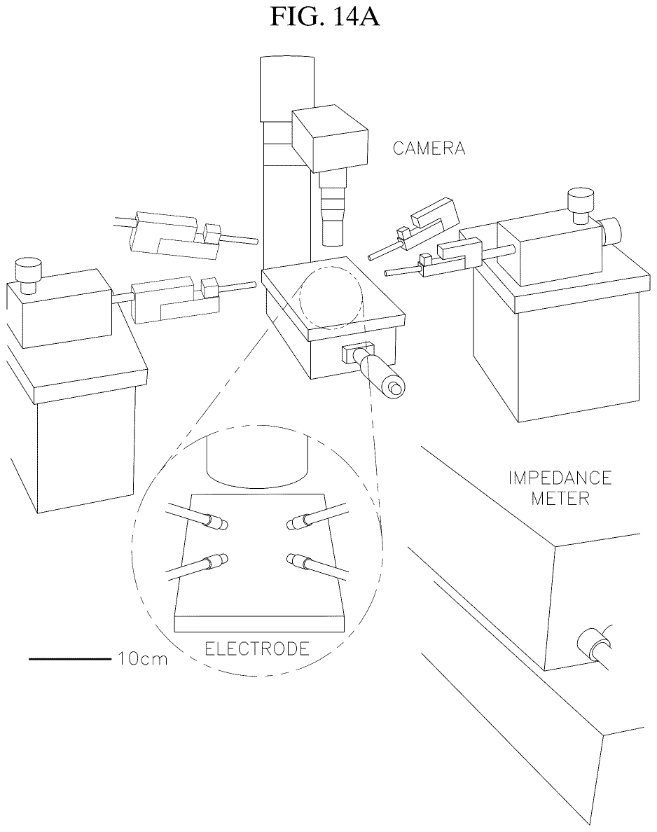

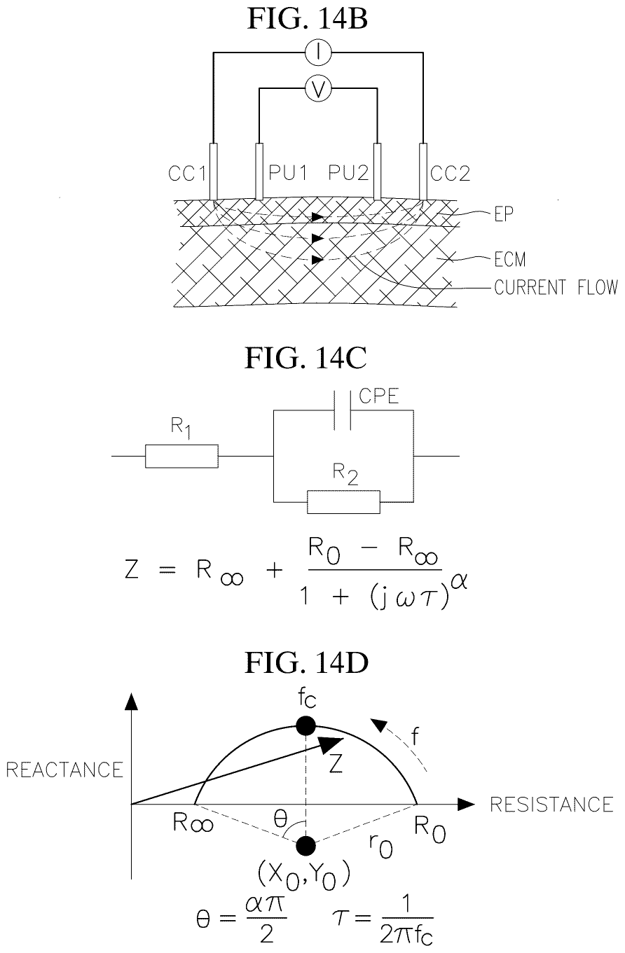

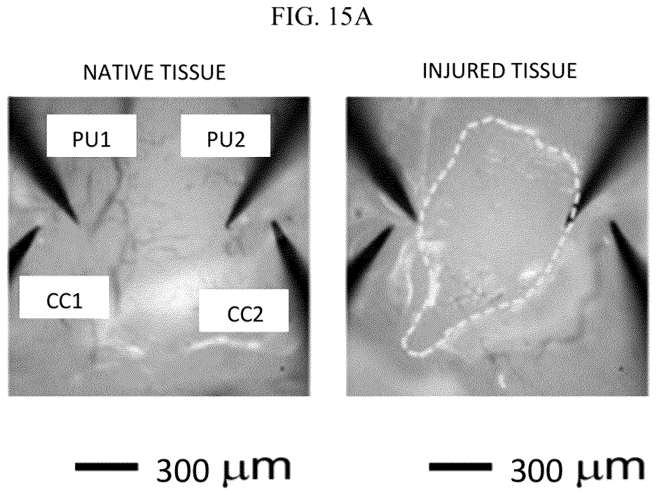

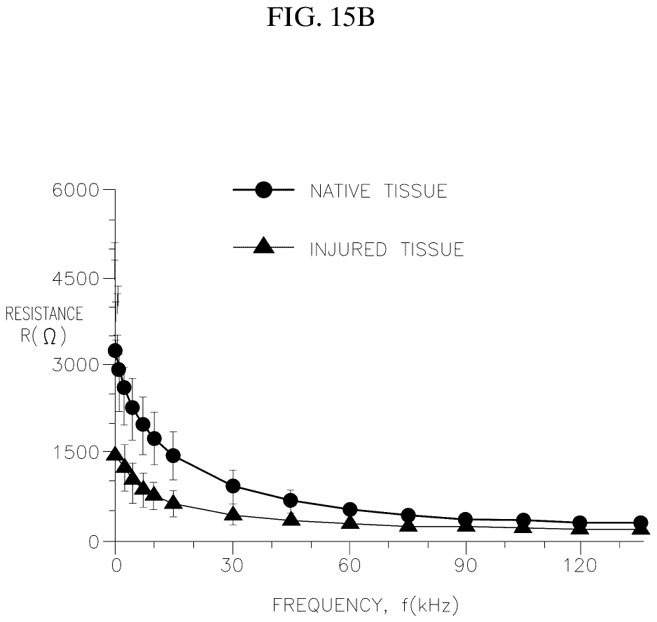

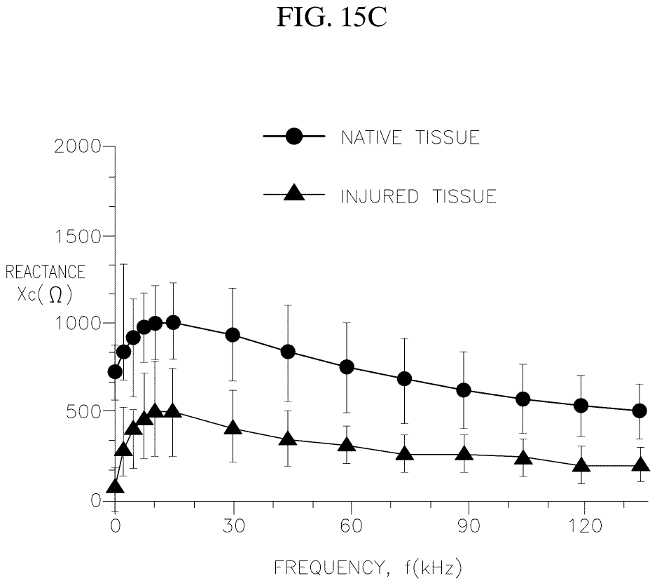

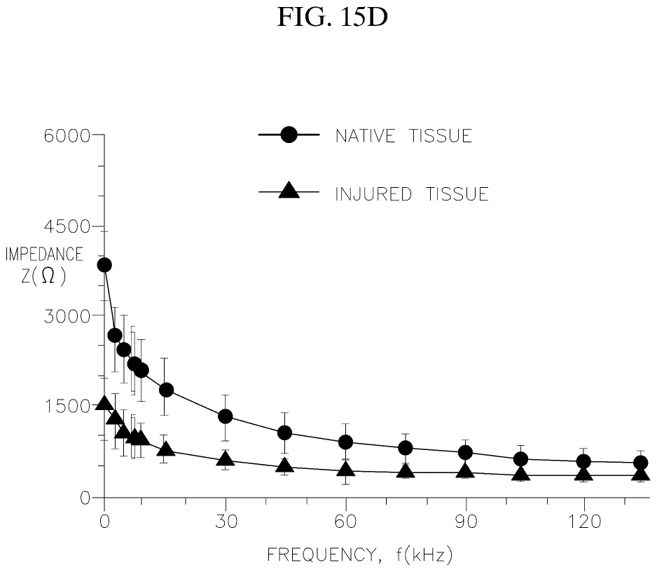

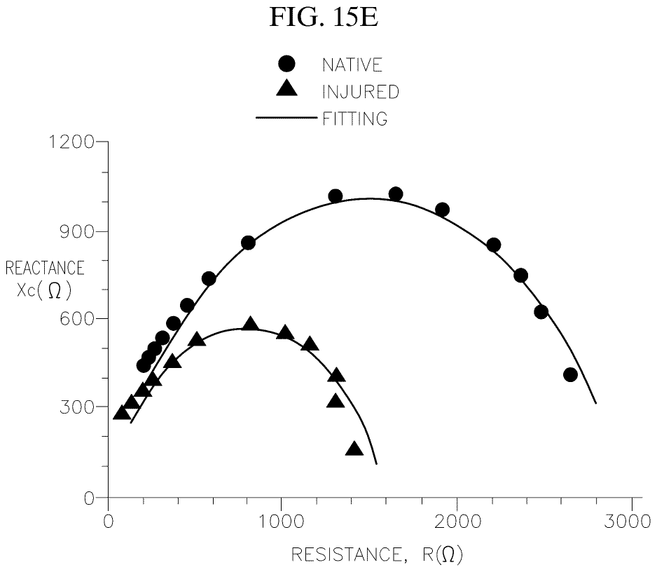

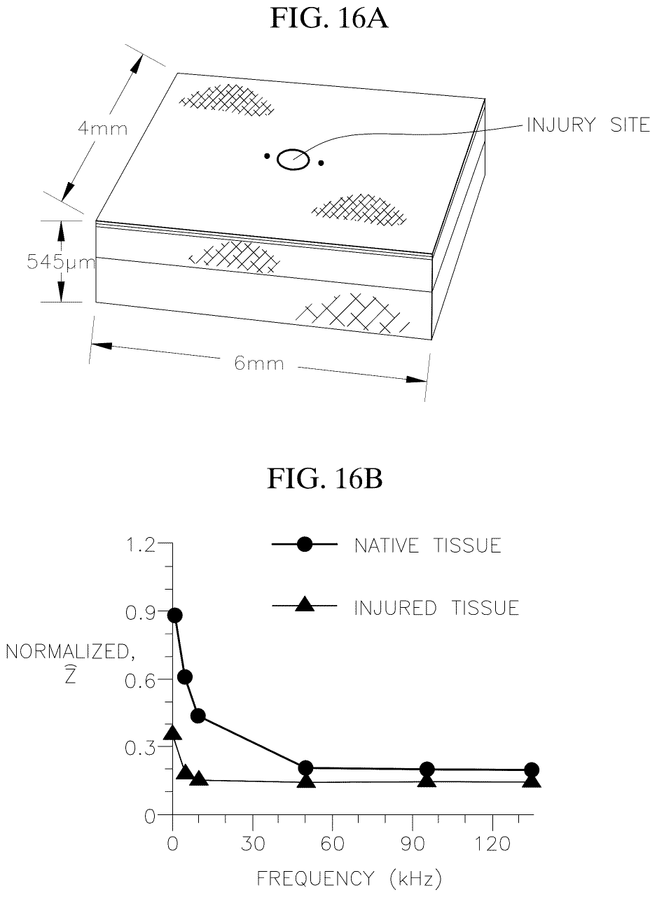

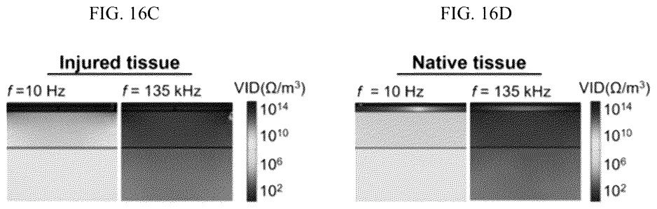

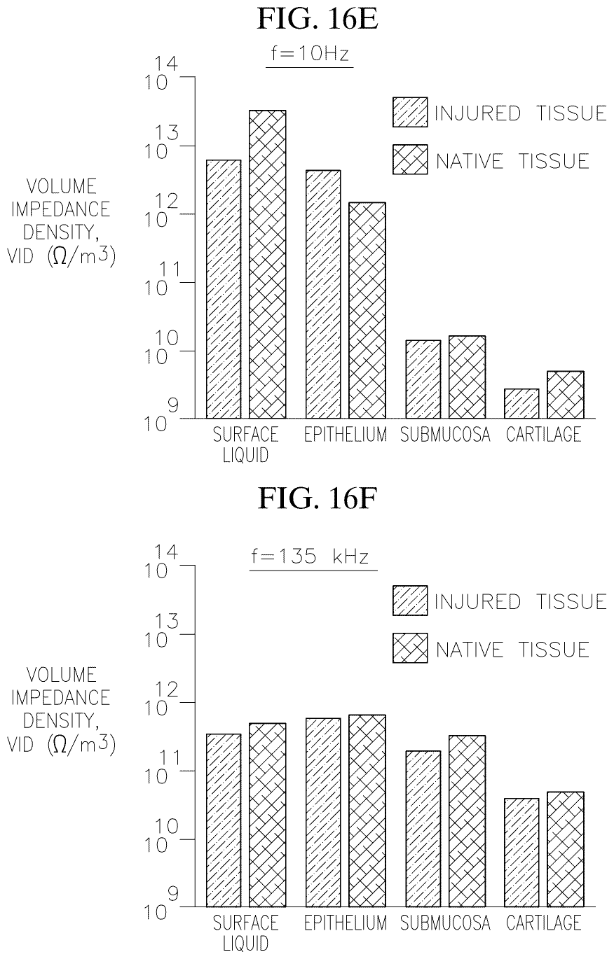

The present invention was developed by investigating acute physical injury generated in the airway epithelium that affects the intrinsic protective functions of the epithelial barrier and whether the functional disruption can be measured directly at the injured tissue regions. Specifically, the impact of injury-induced degradation of the mucociliary clearance and tight/adherens junction formation were quantified, which are the two most essential barrier functions of the airway epithelium. To quantitatively investigate the changes of these epithelial functions following acute injury, in situ particle tracking and bioimpedance measurement techniques (FIGS. 14A-14D and 15A-15E) were combined with mathematical modeling and computer simulation (FIGS. 16A-16F). To enhance reliability of the outcomes, freshly isolated rat tracheal tissues were tested that retained the intrinsic airway tissue architecture and functions, including unidirectional ciliary activities, physiologic cell diversity and distribution, and the native tissue matrix components. Localized acute physical injury was created in a selected region of the isolated tracheal tissues to determine the correlation between structure and function in the airway epithelium with improved spatio-temporal resolution of the measurements. The numerical analyses conducted facilitated elucidation of the structure-function correlation of the airway epithelium by allowing interpretation of the experimentally obtained results.

Notably, the tissue-wise particle tracking revealed that the localized tissue injury substantially degraded the ability of the airway mucosa to transport its surface fluid as the speed of the flow reduced by nearly 60% at the injured tissue regions. Further, the injury resulted in disorientation of the surface flow as the unidirectionality of the flow reduced by at least 10% near the central location of the injury site. Computer simulation of the injury-induced fluid movements showed that the spatial variation of the flow speed near the injury site is likely due to the heterogeneous pressure distribution resulting from the absence of the functioning cilia at the injured tissue. These findings are consistent with a pathological condition where airway tissue regions with substantially impaired mucociliary clearance are vulnerable to subsequent injury and infection due to increased time taken for the inhaled particles or pathogens to be removed from the respiratory tract.

While the flow speed and directionality reduced, transport of the surface fluid could persist at the tissue regions devoid of ciliated cells. This is because the fluid moves as a continuous medium due to intermolecular attractive forces between the fluid molecules. Because of the physical connection between the fluid molecules across the airway surface, the hydrodynamic energy produced by repeated cilia beating at the neighboring intact tissue region can propagate through the fluid due to energy conservation, leading to fluid movement at the injured region. The hydrodynamic crosstalk between the fluids at different locations was clearly visualized via particle tracking at the interface between the intact and injured tissue regions. Due to substantial differences between these two regions in their flow speed and directionality, the surface flow moving over the intact tissue surface caused movement of the fluid from the injury site toward the intact region in the direction perpendicular to the P-D axis of the airway. Propagation of hydrodynamic energy within a fluid is heavily influenced by the fluid properties, such as viscosity, surface tension, and density. Therefore, the maximum size of the injured tissue surface that neighboring cilia can exert their effects on is expected to be dependent on the physical characteristics of the airway surface fluid.

Visualization of the mucociliary flow and cilia motion can be achieved via advanced imaging modalities, in particular optical coherence tomography (OCT). OCT imaging, however, can only provide the cross-section views of tissues or fluids and thus is not suitable for monitoring the two-dimensional (2D) mucociliary flow across large airway surfaces. On the other hand, the present invention can be used in conjunction with a microparticle tracking method, which has been extensively used in the mucociliary-related studies, because of its proven utility in visualization of fluid movements with high spatial and temporal resolution. Tracking the movement of individual microparticles simultaneously along the 2D surface of the injured airway lumen allows for rapid assessment of the impact of localized tissue damage on the mucociliary flow. Further, assessment of the fluid interactions between the injured and intact tissue regions, which can be difficult to visualize using OCT imaging, can be realized via the in-situ particle tracking approach implemented in conjunction with embodiments of the present invention.

It has also been shown that disruption of the tight/adherens junctions between the epithelial cells caused by physical tissue damage can be quantified by measuring the electrical properties of the local airway tissue (FIGS. 14A-14D and FIGS. 15A-15E). Specifically, they show the experimental setup used to measure bioimpedance of the airway tissue (14A), bright field images showing the luminal surfaces of native and injured trachea tissues (15A) wherein the dotted line indicates the injured tissue region. CC1 and CC2: current carrying electrode 1 and 2, PU1 and PU2: voltage pickup electrodes 1 and 2. FIG. 14B is a schematic of the cross-sectional view of the rat trachea with a four-probe bioimpedance measurement system. I: Current source, V: voltage meter, EP: epithelium, ECM: extracellular matrix. FIG. 14C is an equivalent electrical circuit diagram of the rat trachea, known as the Cole model. R1 and R2: resistor 1 and 2, CPE: constant phase element, Z: impedance, R0: resistance at zero frequency, R1: resistance at infinite frequency, a: an empirical exponent (range: 0 and 1), s: characteristic time constant, R₁=R₀, R2=R₀−R_{∞}. FIG. 14D is a Cole-Cole plot that graphically shows different parameters. fc: Frequency corresponding to the maximum reactance. FIGS. 15B-15D are graphs plotting different parameters, including resistance R (FIG. 15B), reactance Xc (FIG. 15C), and impedance Z (FIG. 15D) of both native and injured trachea tissues measured with different alternating current (AC) frequency values (range: 250 Hz-135 kHz). FIG. 15E is a Complex impedance plot of native and injured tracheas. The solid lines represent the best fit to the measured data for the extraction of Cole parameters.

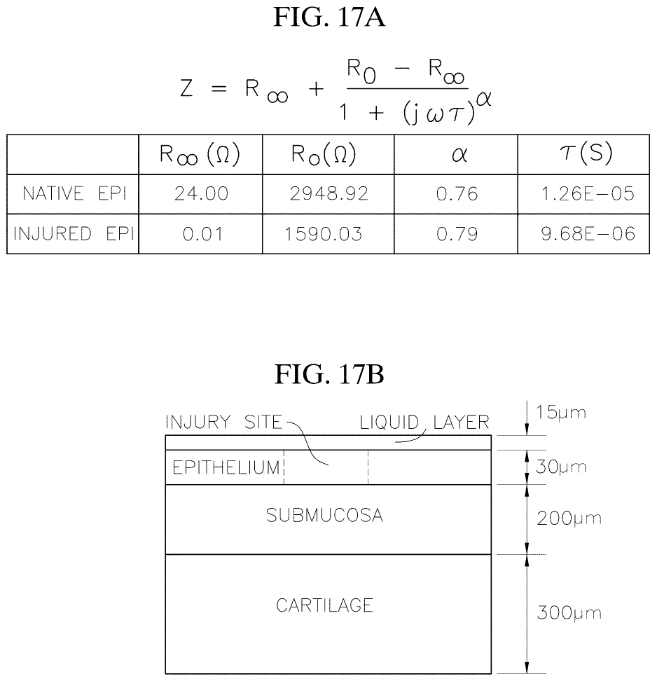

Notably, it has been determined that the acute tissue injury resulted in reduced bioimpedance using the custom-built measurement platform in combination of the mathematical analysis and computer simulation (FIGS. 16A-16F). Loss of the physically protective and electrically resistant epithelial barrier is likely the main contributor of the lowered magnitude of the bioimpedance of the injured airway tissues compared with that of native tissues. Consistent with prior studies, experimental and simulation results show that the magnitude of the electrical resistance of both native and injured tissues decreased with increased AC frequency because the dielectric property of the cell membrane hampered propagation of AC current with lower frequency across the cells. In particular, the simulation results show that the electrical current can propagate into deeper tissue regions when the resistant epithelial layer was removed, suggesting the reduced bioimpedance measured at the injured airway surface is likely due to diffusive distribution of the electrical potential across the larger volume of the airway tissue. Since measuring the electrical characteristics of each tissue layer is very challenging because of their thin geometry (FIG. 17B), the computer model developed can mathematically predict the electrical response of individual tissue components, providing additional quantitative information about the structural integrity of the airway tissue.

The present invention achieves correlation of airway tissue injury with locally measured bioimpedance of affected tissue with an injury size as small as 500 μm. Significantly, the measurement and simulation outcomes suggest that the in situ bioimpedance quantification, when coupled with conventional bronchoscopy, could offer insights into mechanisms of airway epithelial destruction or serve as a diagnostic tool to detect airway injury and disease.

Airway epithelial injury and subsequent impairment of the epithelial barrier functions can be caused by various reasons, such as inhalation injury, trauma, and surgery. Accordingly, the extent of the tissue damage and functional impairment can drastically vary depending on the cause, type, and severity of the injury. In accordance with the present invention, instead of modeling an explicit pathologic condition, physical damage to the isolated airway mucosal tissues was induced to recapitulate the structural and functional disruption in the epithelium that nearly all pathologies of airway injury have in common with varying degrees. The strong correlation between structural damage and functional degradation of the airway epithelium quantified opto-electrically and computationally may facilitate further developments that focus on pathophysiology of specific airway injury models in accordance with embodiments of the present invention. For example, an inhalation injury model can be generated by intratracheally administering acidic fluids to recapitulate epithelial disruption with a larger surface area of pathologic tissue. Such a model can be used to study disruption, repair, or regeneration of the epithelial barrier with respect to severity of the inhalation injury.

Overall, the present invention leverages the relationship between the structural integrity and protective barrier functions of the airway epithelial layer. The in-situ flow visualization and simulation achieved via tissue-wise particle tracking and computational modeling can collectively allow rapid analysis of the transport phenomena of the airway surface fluid. Direct measurements, coupled with computer simulation, of the electrical properties of both healthy and injured airway tissues can promote label-free, minimally invasive detection and quantification of airway tissue injury. Collectively, the present invention offers innovative tissue monitoring tools and computational models that can facilitate both fundamental and translational developments associated with diagnosis and treatment of airway tissue injury.

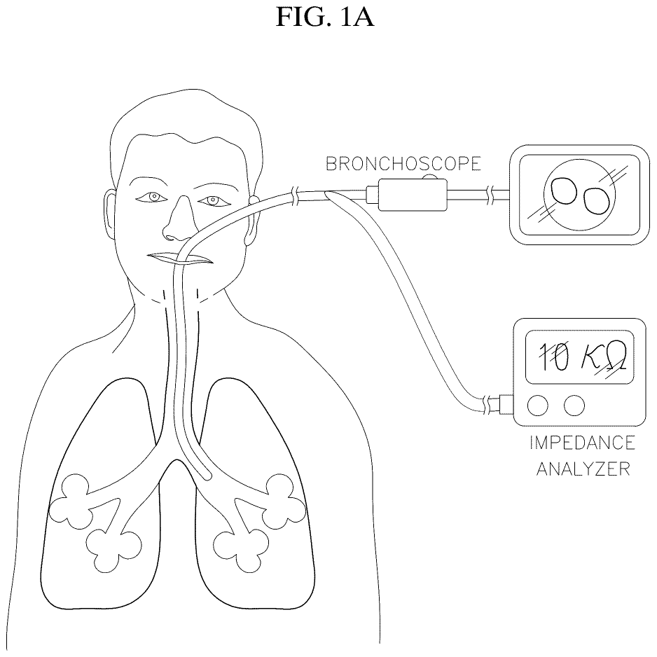

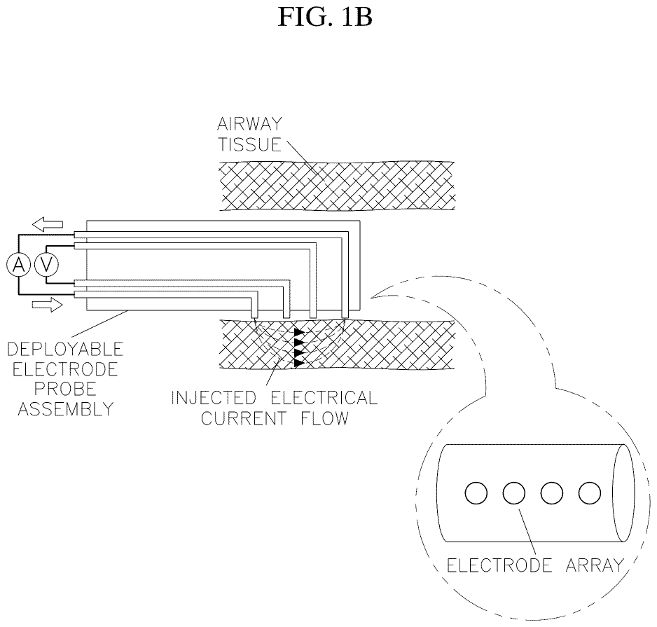

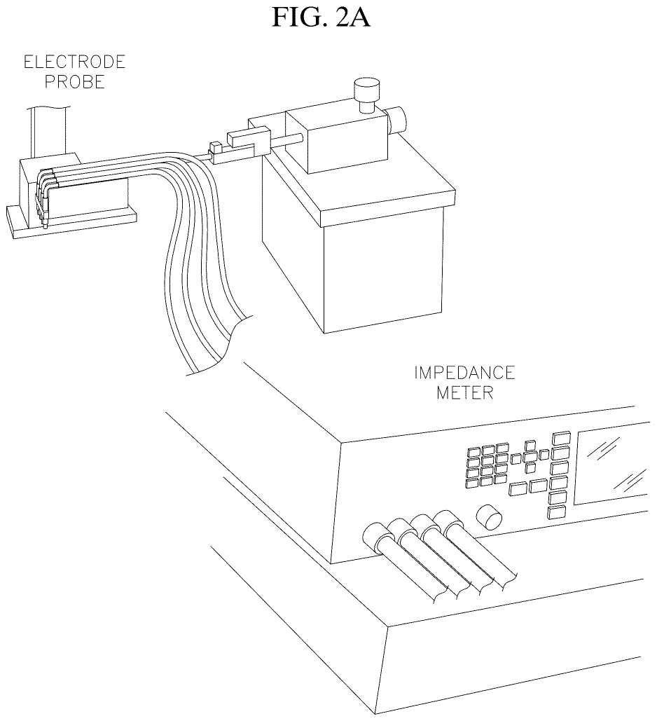

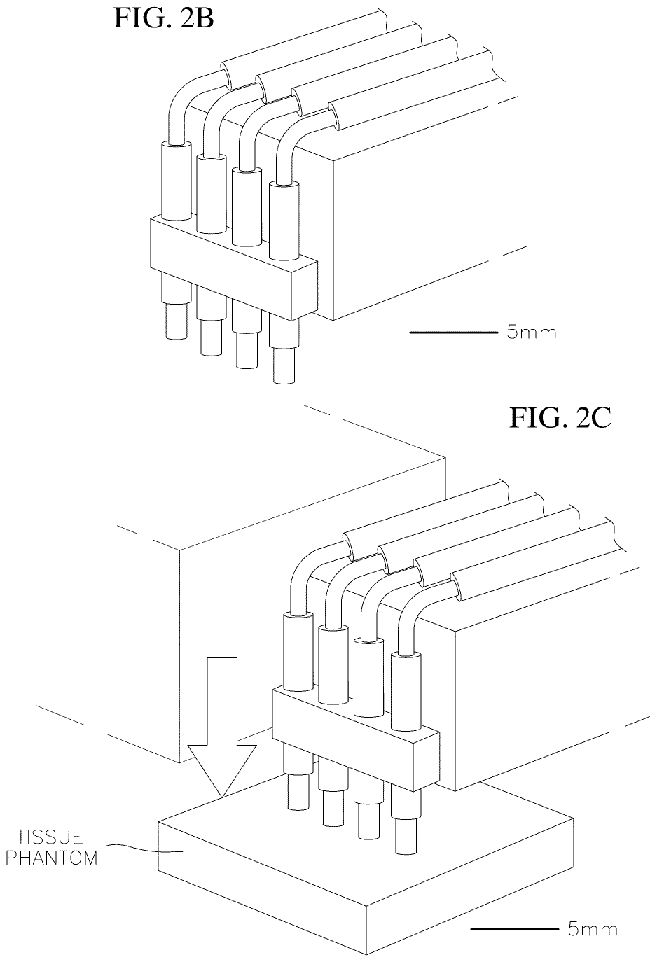

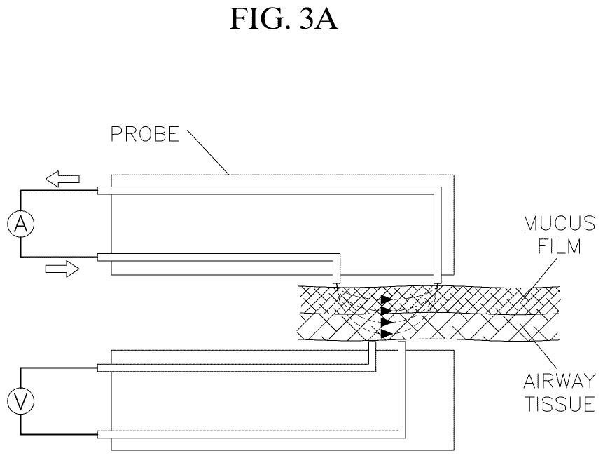

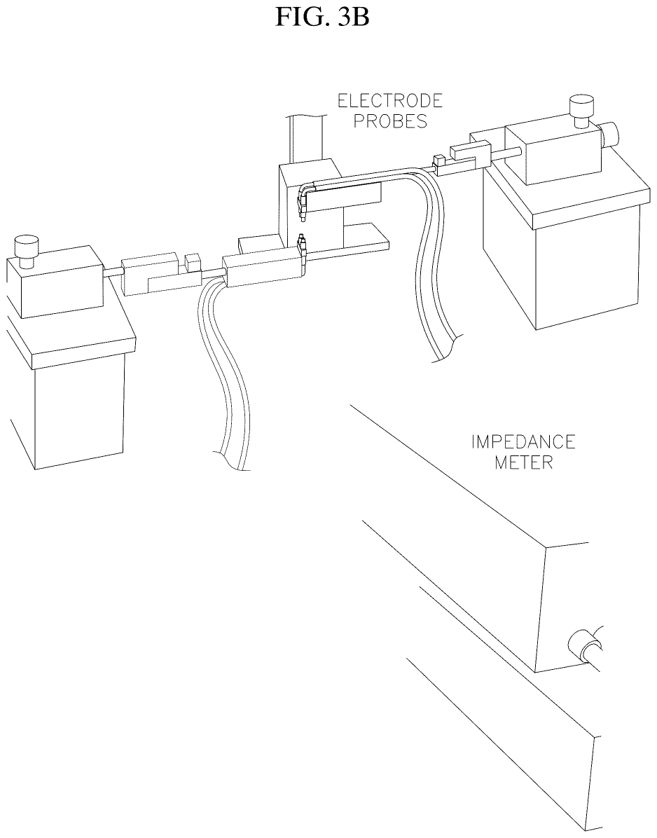

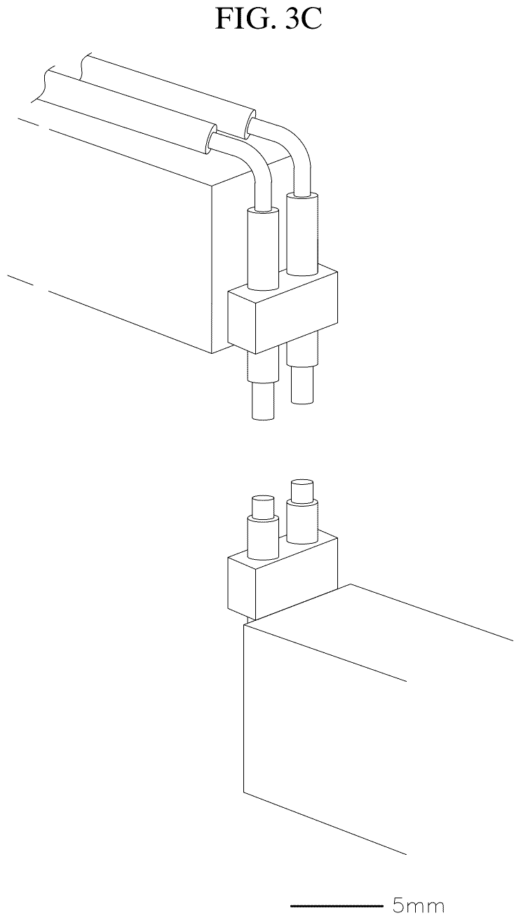

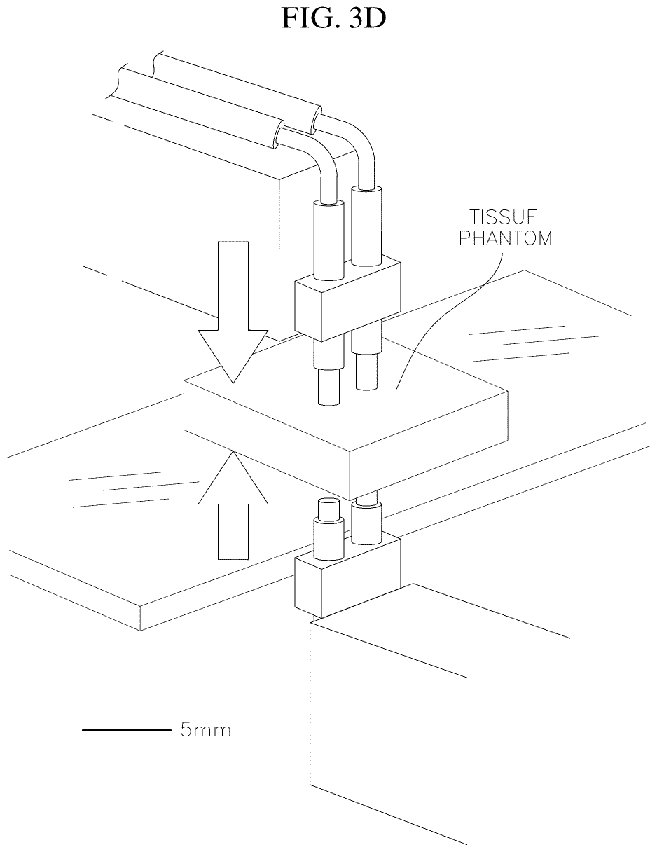

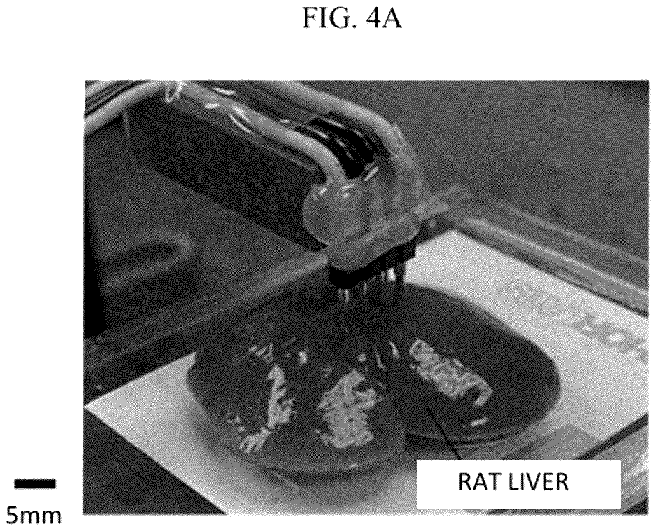

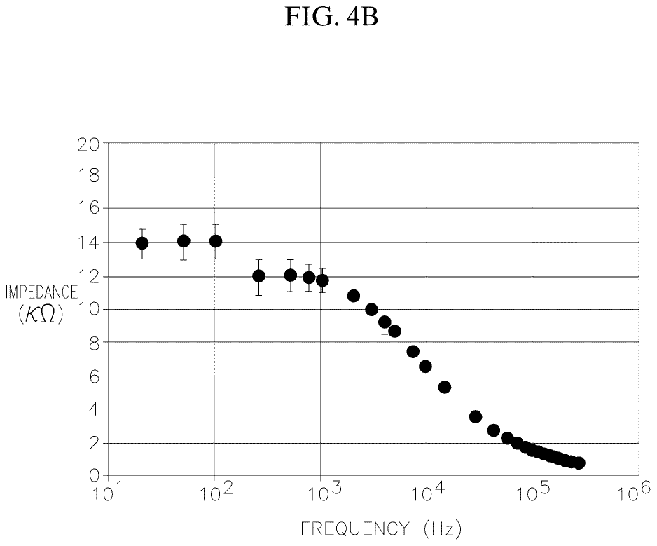

Referring now to FIG. 1A, for the in situ electrical impedance measurements, a deployable electrode assembly can be inserted intratracheally into the respiratory tract of the patient's lung via a video bronchoscope (see FIG. 1A). The distal end of the electrode assembly is integrated with an array of electrodes that can directly contact the surface of the airway lumen for impedance measurements in a localized manner. The proximal end of the device may be connected to an impedance meter to determine the local airway impedance values. The electrode array is arranged in a way that it can inject electrical current into the airway tissue or pulmonary liquid while measuring the electrical impedance (see FIG. 1B). A prototype of the deployable electrode assembly and impedance measurement system (see FIGS. 2A and 2B) includes four electrodes at the distal end which are connected to an impedance meter (e.g., Keysight E4980A). The system can be used to determine the impedance of different tissue phantom (see FIGS. 2C, 3D) and biological tissues in vitro (see FIGS. 4A-4B, 15A-15E, 21A-27I). The impedance measurements could be also performed by applying the electrical current to the luminal surface and measuring the voltage from the outer airway tissue. Such an arrangement could be useful for measuring impedance of in vitro-cultured airway or other biological tissues (FIG. 3).

It was investigated whether the localized damage created in the airway epithelium can be detected via measurement and analysis of the electrical property at the injury site (FIGS. 14A-14D). To this end, a measurement platform was created that allowed for the quantification of bioelectrical impedance (i.e., bioimpedance) of the airway tissue (FIG. 14A). This platform utilizes the four-point measurement approach, in which four electrode probes directly contact the airway tissue to measure the electrical properties of the tissue. During the measurements, alternating electrical current (AC) with various frequencies (range: 20 Hz 135 kHz) and 100 μA of its maximum amplitude was injected into the tissue using two current carrying electrodes (CC1 and CC2), while two voltage pick-up electrodes (PU1 and PU2) were measuring the potential distribution at the measurement site (FIG. 14B). The data acquired was then analyzed using the Cole model that describes the biological tissue as a combination of two resistors (R1=R∞ and R2=R0-R∞) and a constant phase element (CPE) (FIGS. 14C, 14D).

More specifically, the hypothesis that disruption of the airway epithelium leads to reduced bioimpedance of the airway tissue measured at the airway lumen was tested (FIGS. 15A-15E). To test this, a focal epithelial injury was created using a metal rod, and the four electrodes were placed near the injury site (FIG. 15A). The measurement results showed that the magnitude of both resistance (R) and reactance (Xc) measured at the injured tissue were noticeably lower than those measured at the native tissue region (FIGS. 15B, 15C). The magnitude of the reactance increased with increased AC frequency (from 0Ω to ~500Ω in injured tissue; from 0Ω to ~1,000Ω in native tissue) and then gradually decreased as the frequency further increased after a certain threshold frequency (fc: ~10 kHz in injured tissue; ~15 kHz in native tissue) (FIG. 15C). The bioimpedance (Z) of both injured and native airway tissues reduced gradually as the AC frequency increased (FIG. 15D). Each data set acquired from injured and native tissue was fitted to a curve by the least absolute deviation (LAD) method, from which the Cole parameters were extracted (FIG. 15E; FIG. 17A). Notably, the resistance values were measured for DC current (R₀) and infinite AC current (R_{∞}), and the time constant (r) acquired from the injured tissue were smaller than those acquired from native tissue, which is likely due to damaged airway epithelial barrier at the injury site.

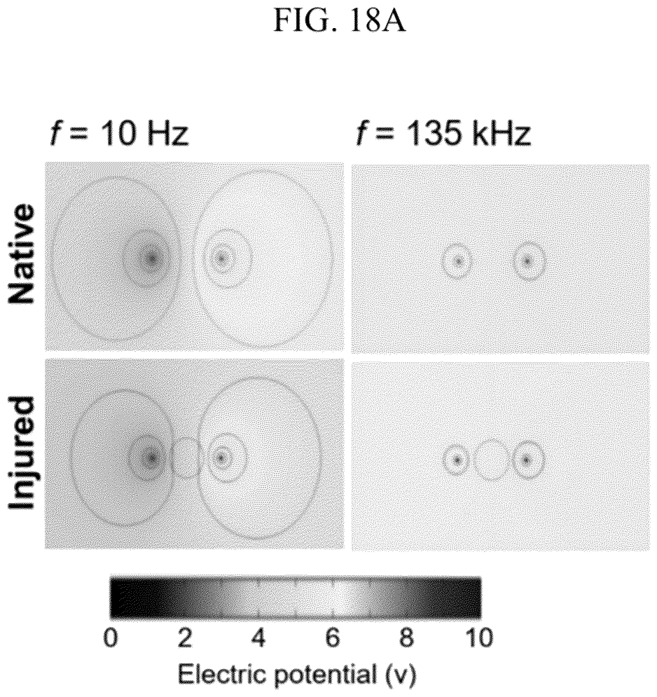

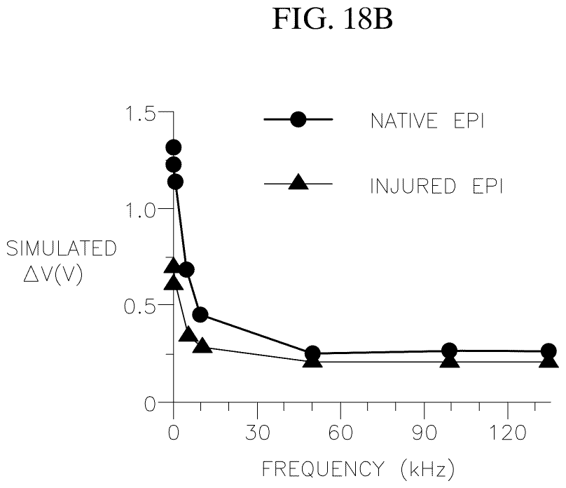

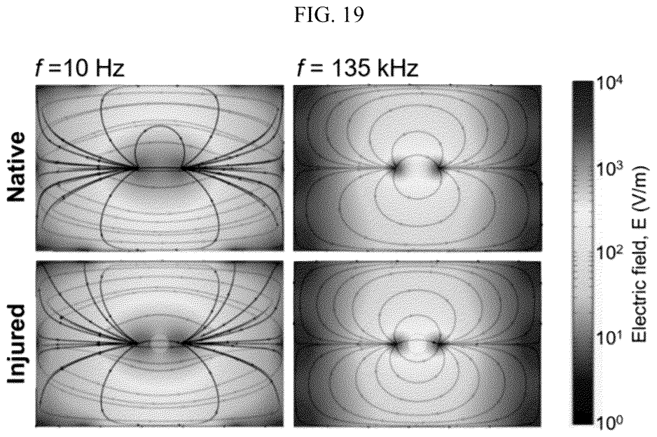

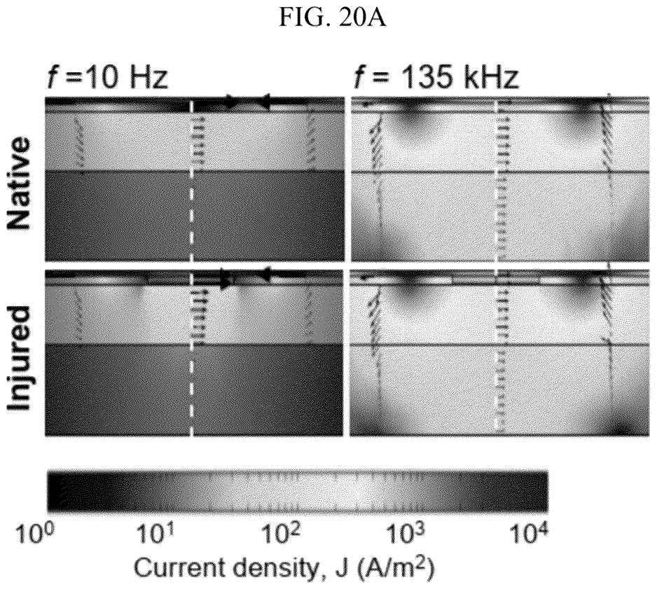

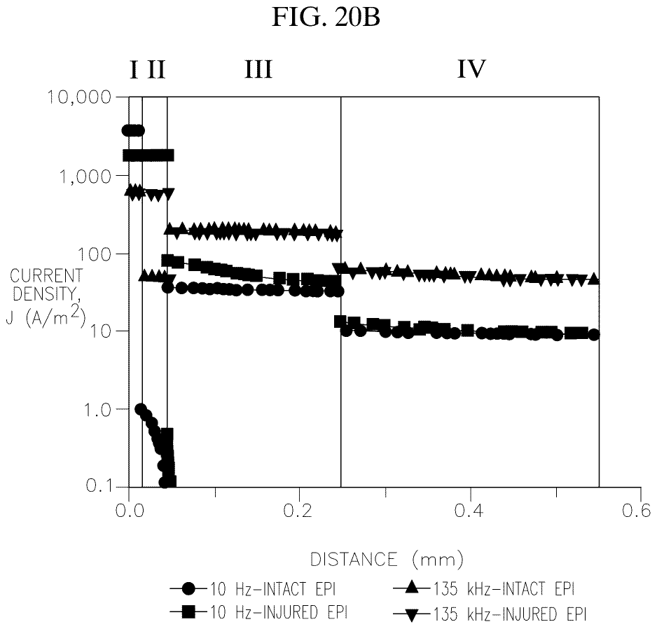

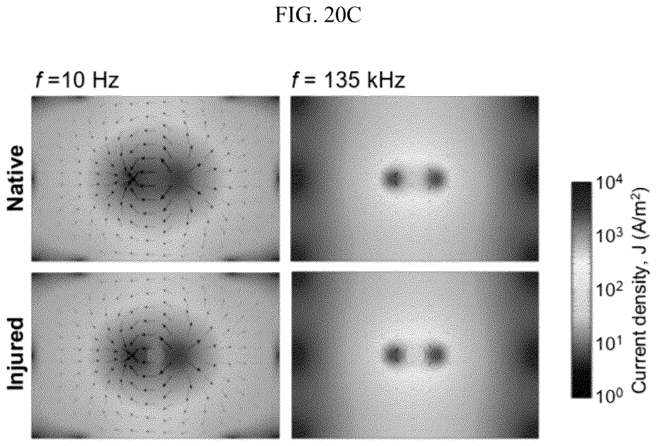

To rationalize the contribution of the airway epithelial injury in the alteration of the electrical property of the airway tissue, a numerical simulation of the electrical current propagation through both injured and native airway tissues was conducted (FIGS. 16A-16F). The airway tissues were modeled as a composite of tissue layers that consisted of the epithelium, submucosa, and cartilage. Further, the computer model included a thin layer of pulmonary surface liquid (thickness: 15 μm) deposited on top of the epithelial layer (FIG. 16A; FIG. 17B). The simulation results showed that the potential difference (ΔV) (FIGS. 18A-18B) and normalized bioimpedance (Z′) (FIG. 16B) between the voltage pick-up electrodes (PU1 & PU2) decreased with increased AC frequency. Further, these values measured at the injured tissue were generally smaller than those measured from intact regions, which were consistent with experimental results (FIGS. 15A-15E). Contour plots of the simulated results showed that the distribution of the electric field (E) (FIG. 19) and current density (J) (FIGS. 20A-20C) throughout the tissues were substantially affected by the AC frequency supplied to the tissue. While the electrical current propagates through the superficial tissue near the epithelium at lower AC frequency, the current can penetrate deeper tissue regions as it travels through the tissue at higher AC frequency.

To investigate relative contribution of each tissue layer in the electrical measurement outcomes, volume impedance density (VID) of different tissue layers was computed in both injured and native airway tissues (FIGS. 16C-16F). Since VID is defined as the magnitude of the bioimpedance per unit tissue volume, it can provide information about the relative role of different tissue layers in resistance to the electrical current. At low AC frequency (e.g., 10 Hz), the VID value in the surface liquid was three to four orders of magnitude greater than that in the submucosa or cartilage layer in both injured and native tissues (FIG. 16E). The VID value of the surface liquid in injured tissue (6.58×1012 Ω/m3) at 10 Hz was much smaller than that in the native tissue (3.36×1013 Ω/m3), due to easier penetration of electrical current through the damaged epithelium than the intact epithelium. The difference of VID between the superficial and distal tissue regions became less significant at higher AC frequency (e.g., 135 kHz), suggesting low AC frequency could generate more robust and reliable electrical readouts when characterizing the tissue injury that occurs at the superficial airway tissue layers (FIG. 16F).

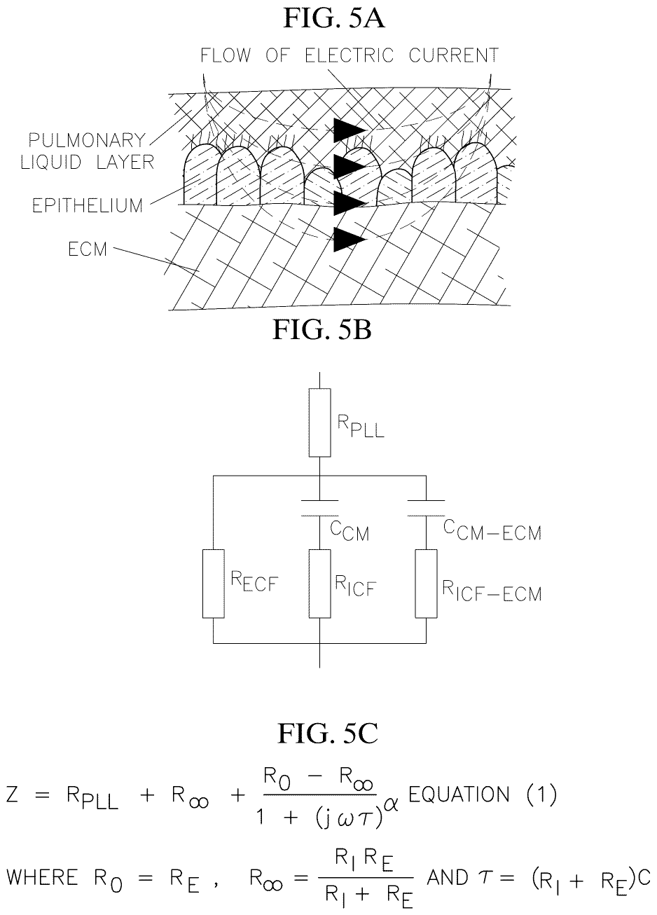

Using a similar system, isolated rat liver was used for impedance measurements while varying the frequency of AC current from 30 Hz to 300 kHz. As the frequency increased, the impedance of the tissue decreased because higher frequency current can pass through the cells more easily (FIG. 4). The injected electrical current can propagate through the pulmonary liquid and tissue layers (FIG. 5A). The flow of electrical current can be represented by an electric circuit that includes the resistance of pulmonary liquid layer (RPLL), the resistance of the extracellular fluid (RECF), the resistance of the intracellular fluid (RICF), the resistance of the intracellular fluid within ECM (RICF-ECM), the capacitance of the epithelial cell membrane (CCM), and the capacitance of the cell membrane within the ECM (CCM-ECM) (FIG. 5B). With reference to FIG. 5C, the impedance (Z) of the airway tissue coated with mucus film can be calculated using Equation (1), where R0 is the resistance measured with zero frequency (i.e., DC) and R∞ is the resistance measured with infinite frequency AC. As the equation indicates, the total tissue impedance measured will be proportional to the resistance of the mucus layer.

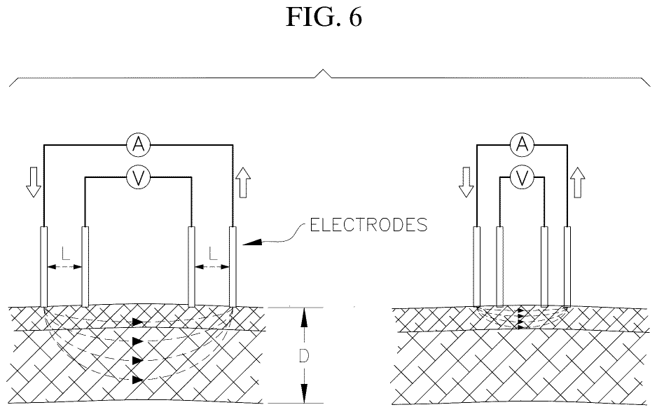

The tissue penetration depth of injected electrical current depends on measurement parameters, such as the frequency of the alternating current (AC), spatial arrangement of the electrodes, and size of the electrodes contacting the tissue. Thus, by modulating the measurement parameters, the impedance of the airway tissue at different depths could be determined. For example, by adjusting the distance between electrodes (L), the penetration depth of injected current (D) can be modulated, thereby allowing impedance measurements at different tissue depths (FIG. 6).

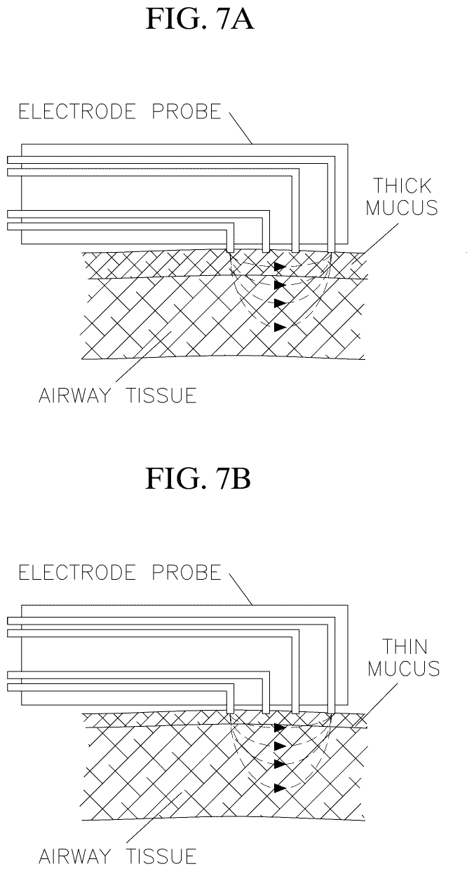

The airway impedance measurement device can be used to (i) monitor airway health and disease or (ii) as a tool to facilitate therapeutic delivery of cargo, including gene therapy. For example, the device can be used to determine the presence or absence of the mucus layer lining on the airway lumen of the CF patient's lung. Because a thick mucus layer will increase the overall impedance of the airway tissue, the airway lumen with the mucus film removed will be indicated by reduced impedance (FIGS. 7A-7B).

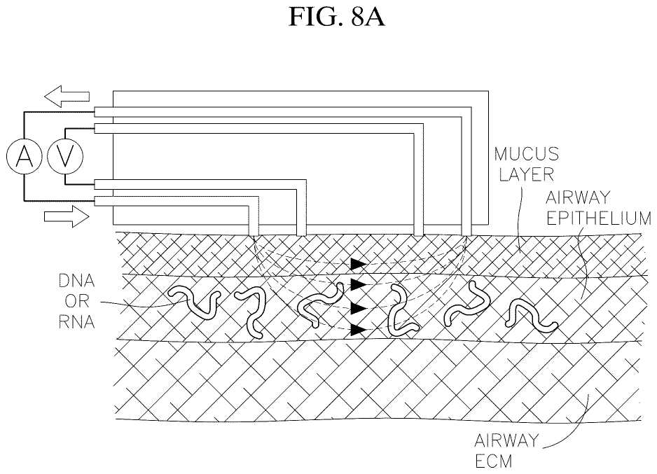

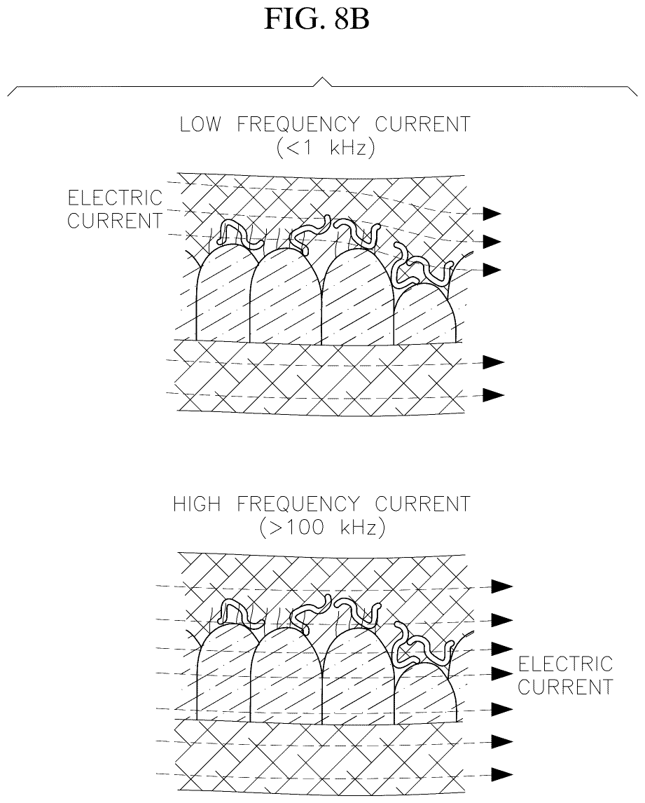

Furthermore, by modulating the measurement parameters, such as AC frequency and electrode configuration (FIG. 5), the location of the gene (or gene editing machinery) within the airway tissue could be determined (FIG. 8A). Because DNA or RNA strands used for gene editing are electrically negatively charged, their spatial distribution across the airway tissue can be indicated by alteration in the electrical impedance values measured with varied measurement conditions, such as AC current frequency and probe arrangement (FIG. 8A). Low frequency current (<1 kHz) will tend to travel through the extracellular fluids due to high impedance imposed by the cell membranes, while higher frequency current (>100 kHz) can pass through the cell membranes. Thus, DNA or RNA remaining outside the cells could be detected by impedance measured at low current frequency and those internalized into the cells can be detected by the impedance measured at high frequency electrical current (FIG. 8B).

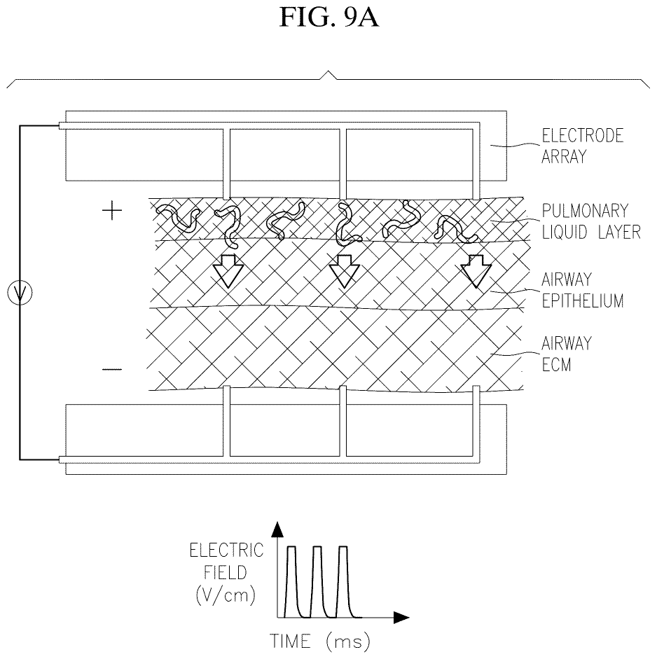

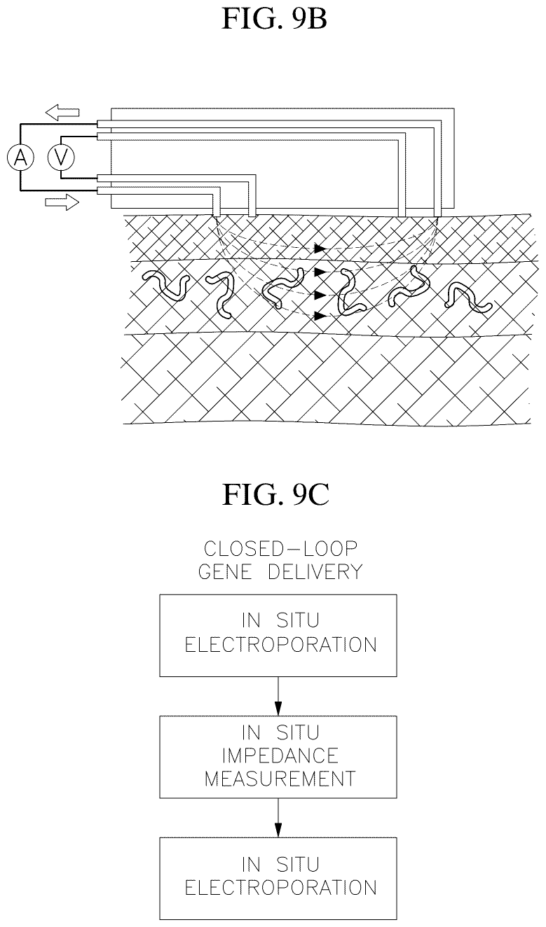

Referring to FIG. 9A, the electrode assembly can be used to provide a pulse of high-voltage electricity (e.g., pulse duration: 150 ms; electric field strength: 200 V/cm) across the airway tissue to facilitate internalization of the genes via electroporation into the internal space of the epithelial cells. In this approach, the genes (or gene editing machinery) can be deposited onto to the airway lumen and are exposed to the electrical pulse for the internalization (FIG. 9A). Via the impedance measurement, spatial distribution of the genes can then be estimated (FIG. 9B). When needed, the electroporation step can be repeated to maximize gene internalization (FIG. 9C).

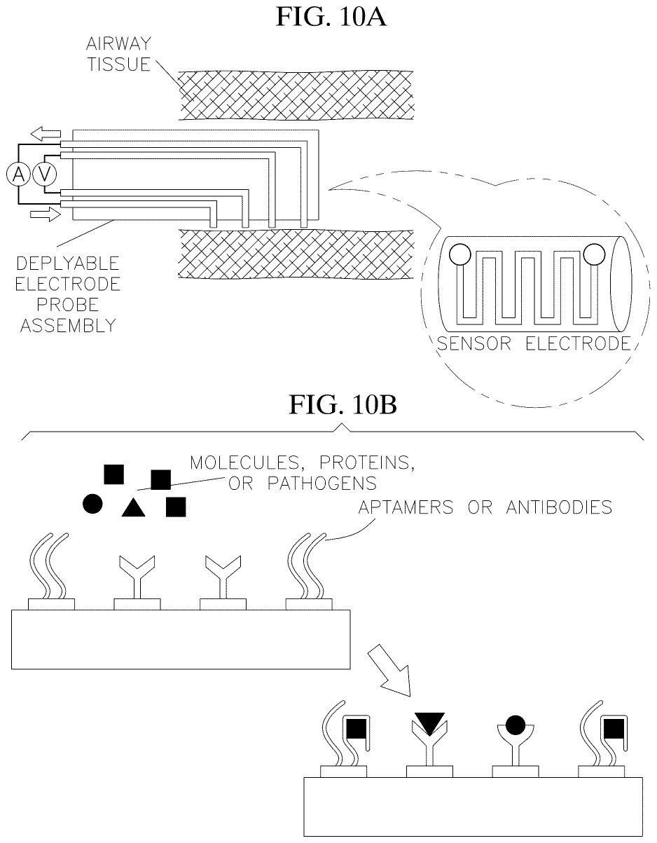

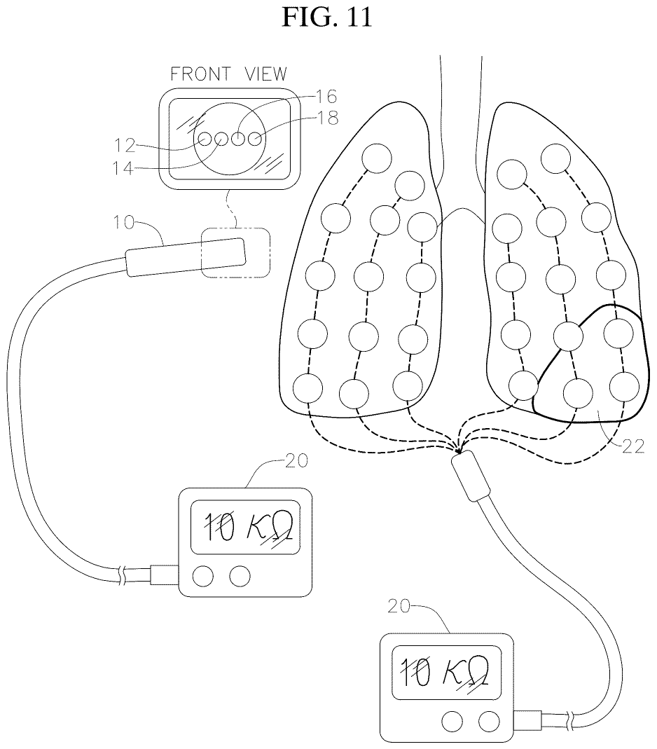

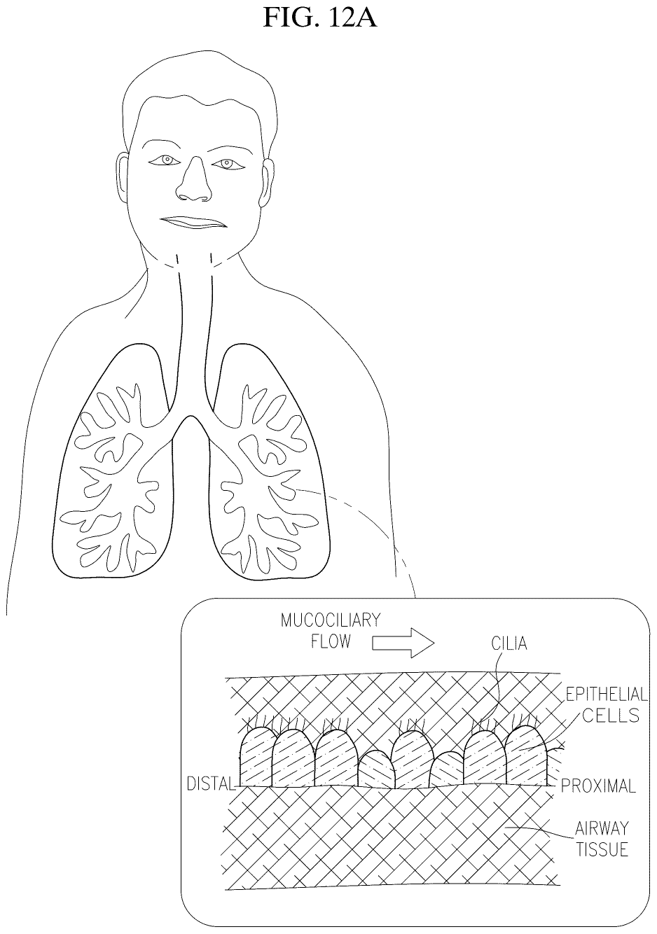

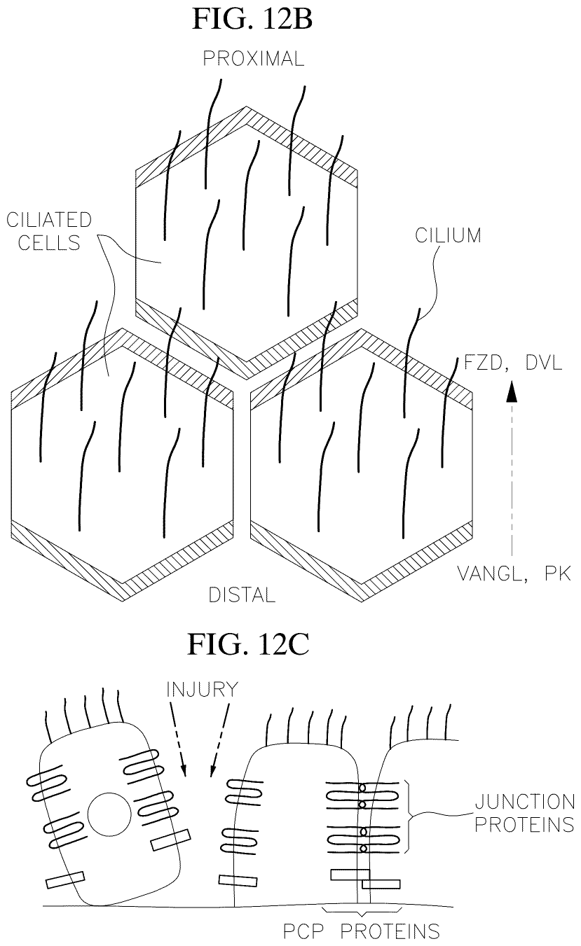

Additionally, charged biological material (e.g., bacteria, biofilms, cell-free DNA, matrix protein fragments, etc.) can be present in the airway and may correlate with disease state or severity. Therefore, the electrode biosensor system described above can also enable the detection and assessment of these markers of disease state. Furthermore, the electrode can be designed in a way that its surface can be functionalized with molecules or proteins, such as aptamers or antibodies, that can recognize and capture specific target molecules, proteins, or pathogens associated with airway injury or disease. Binding of these molecules, proteins, or pathogens onto the sensor surface can result in changes in the electrical properties of the sensor that can allow diagnosis or prognosis of airway tissue injury or disease (FIG. 10).

Also, the impedance-based assessment of donor organs at the time of organ donation or during ex vivo organ support or reconditioning can more accurately quantify the quality of the organ by using a hand-held electrode probe or a wearable probe array. For example, locations of the pulmonary edema, fibrotic or diseased tissues, or tissue integrity in donor lungs or any other organs, such as the liver, can be determined by locally measuring the electrical impedance. In donor lungs, the airspace of the lung filled with fluid (i.e., edema) will display decreased impedance compared with air-filled regions because fluid is more electrically conductive than air. Fibrotic tissue will display increased impedance due to the fact that its dense tissue is more resistant to the electrical current (FIG. 11). FIG. 11 illustrates probe 10 with electrodes 12, 14, 16, 18 connected to impedance analyzer 20, in accordance with embodiments of the present invention to measure injured, damaged or diseased tissue 22.

### Example 1

Lungs were isolated from Sprague-Dawley rats (SAS SD Rats; Charles River Laboratories; body weight: 200-250 g). All animal experiments were approved by the Institutional Animal Care and Use Committee (IACUC) at Stevens Institute of Technology. Briefly, animals were euthanized, and lungs were isolated and rinsed with 1□PBS prior to the bioimpedance measurements. Then, rat lungs were connected to a small animal ventilator (PhysioSuite, Kent Scientific) through the endotracheal cannula and ventilated with a tidal volume of 7 mL/kg at a rate of 70 breaths per minute (bpm). To induce injury in the lungs caused by acid, different volumes (1, 3, and 5 mL) of hydrochloric acid (HCl) solution (0.1 N, pH level: 1.5) were intratracheally instilled into the lung via a 10 mL syringe connected to the trachea cannula. After each HCL instillation, 0.5 ml of air was injected through tubing to ensure that HCl had reached the distal parts of the lungs. The impedance values were measured with the four-probe method with alternate current (AC) using an LCR meter (Keysight Technologies). The impedance analyzer was set to apply a sinusoidal current of 100 mA as the electrical excitation for all the measurements. The frequency sweep measurements were done in the frequency range from 100 Hz to 300 kHz, and resistance (R, real part of impedance) and reactance (X, imaginary part of impedance) were collected. The impedance (Z) values were calculated from resistance and reactance values using the following equations:

Z=R+jX and Z□R□X(□)

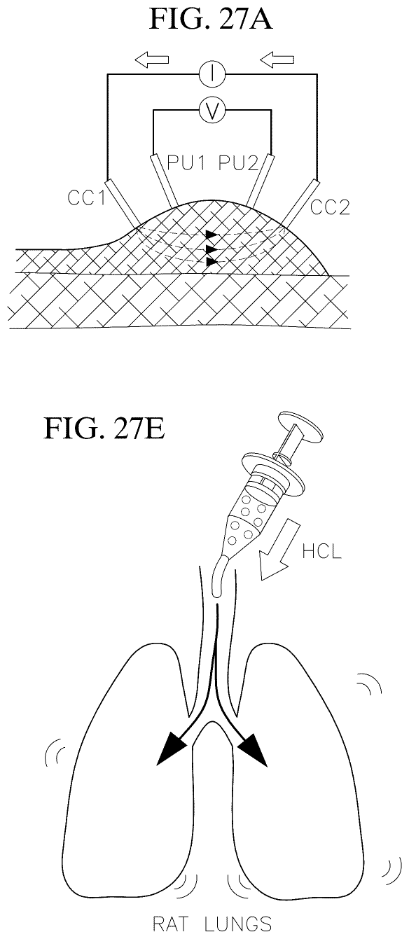

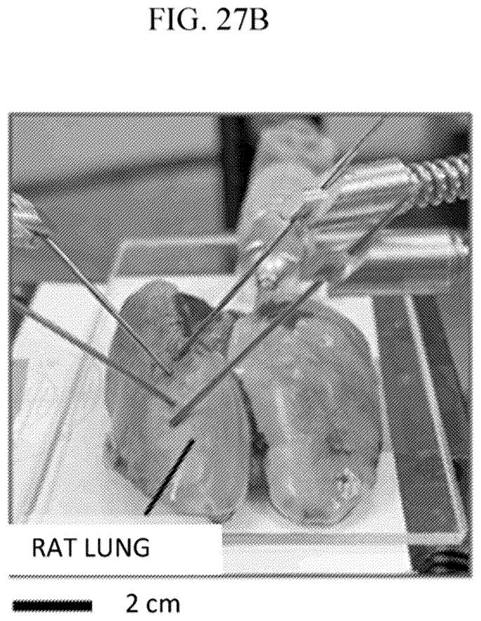

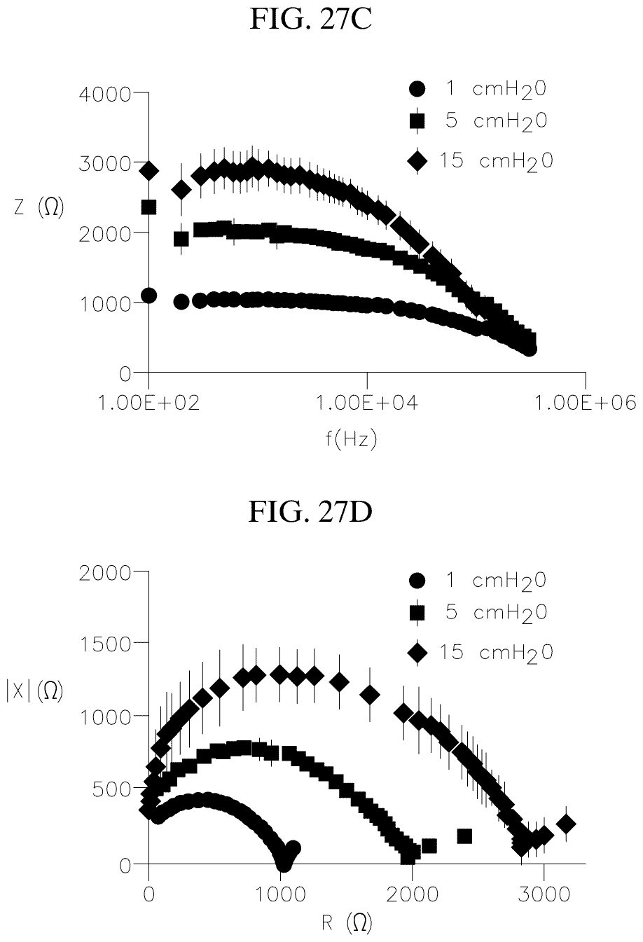

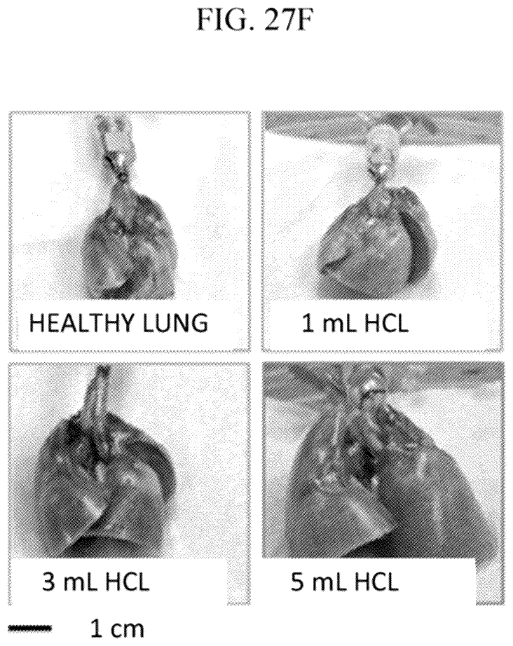

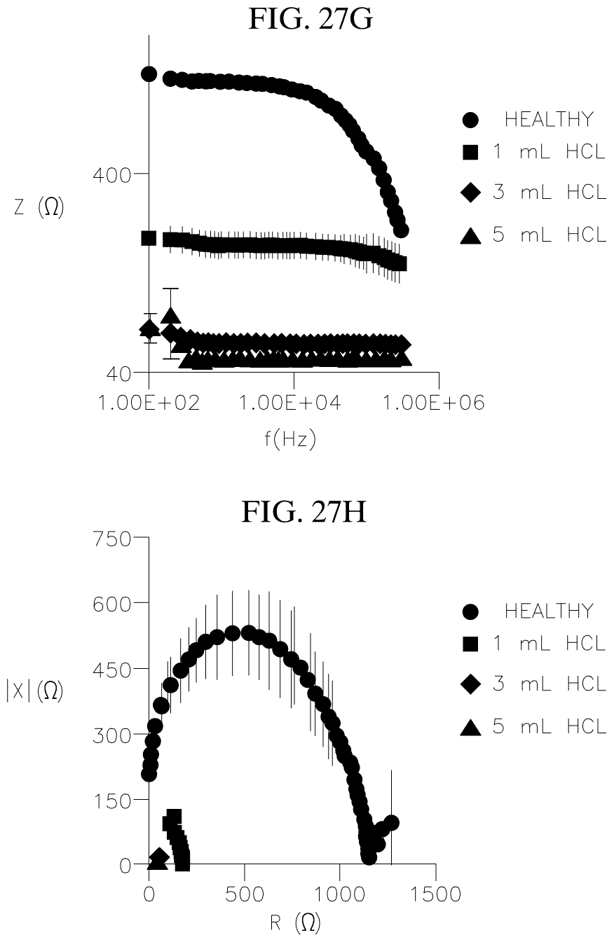

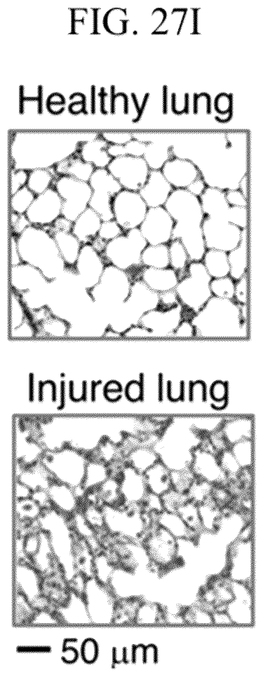

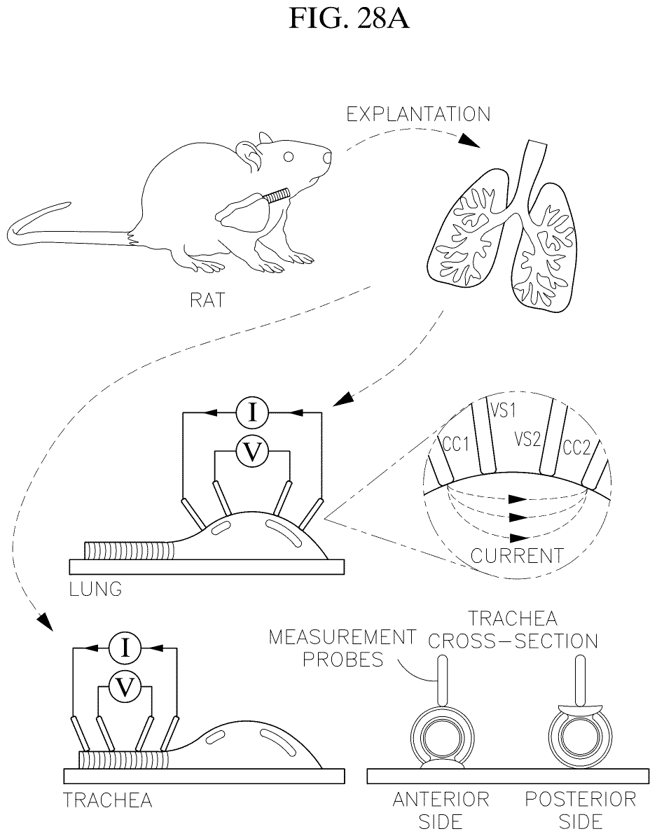

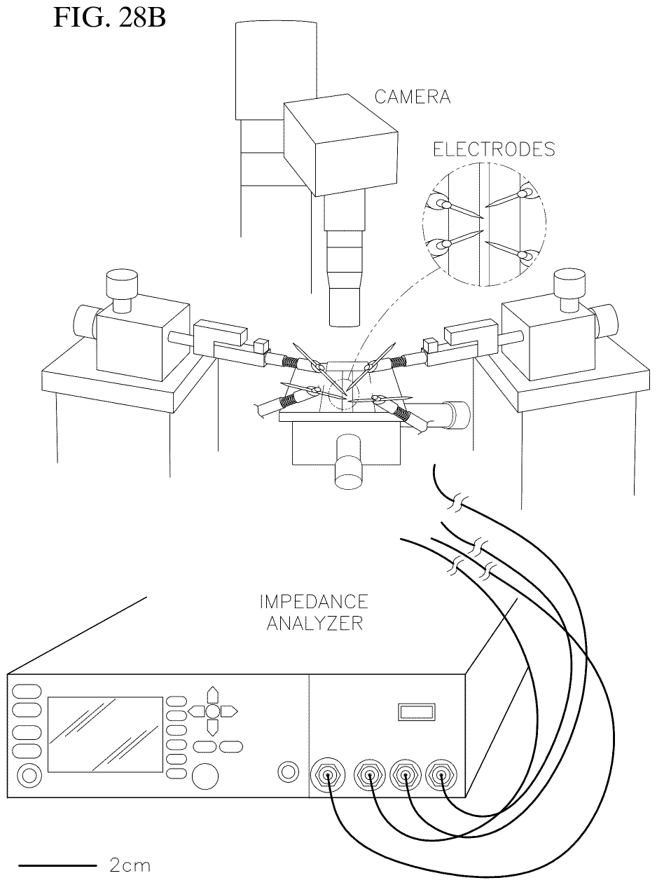

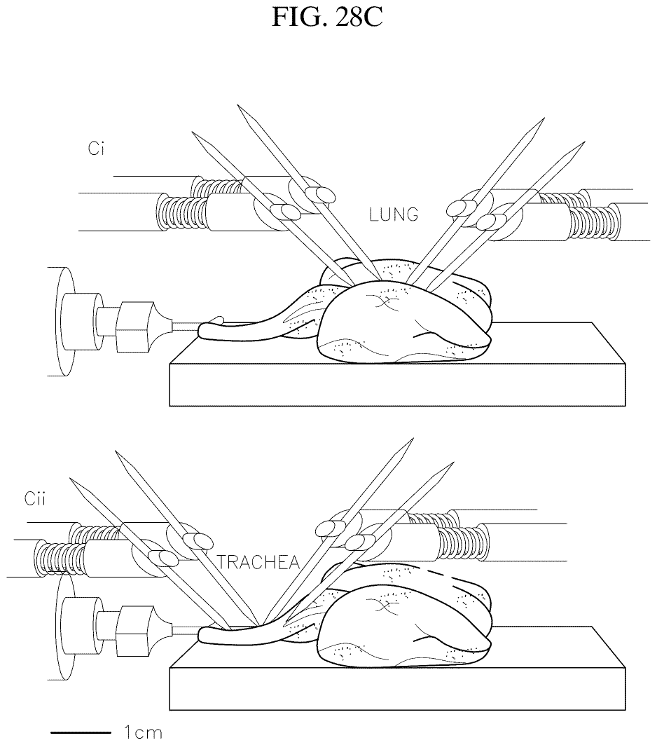

To assess the functionality of the bioimpedance measurement setup, the electrical impedance of the rat lungs ex vivo was evaluated at with different peak inspiratory pressures (PIP: 0, 5, and 15 cmH2O) (FIGS. 27A-27D). An alternating electrical current (AC) with various frequencies (range: 100 Hz-300 kHz) and a maximum amplitude of 100 μA were injected into lung tissue using two current-carrying electrodes (CC1 and CC2), while two voltage pickup electrodes (PU1 and PU2) measured the voltage at the measurement site (FIGS. 27A, 27B). The frequency-sweep curves showed the same pattern for electrical impedance with the change in frequency (FIG. 27C). Moreover, the impedance was increased with increasing the lung's internal pressure due to stretching of the lung tissue and narrowing of the electron/ion conductive pathways with increasing PIP (FIGS. 27C, 27D).

It was further investigated whether the electrical impedance of the lung could be altered by creating an acid-induced lung injury model in the lungs (FIGS. 27E-27I). Intratracheal instillation of HCL solution (concentration: 0.1N), a commonly used solution to model gastric aspiration-induced lung injury, was achieved to create the pathological condition (FIGS. 27E, 27F). These results indicated that HCL-induced injury led to a decrease in the electrical impedance of the lung (FIGS. 27G-27H). The reduction of electrical impedance in injured lungs was connected to the replacement of air with liquid in air space and an increase in the alveolar septa thickness due to tissue dissociation, which collectively provide more conductive pathways for electron/ion mobility. Notably, histological images of the injured lungs (5 mL HCL) confirmed excessive accumulation of liquid and debris in the alveolar airspace and increased thickness of the alveolar septa (FIG. 27I).

Overall, it was shown that bioimpedance assessment of lung tissue can be used as a diagnostic tool for minimally invasive detection and quantification of pathological lung tissue, such as acid-induced lung injury.

### Example 2: Bioimpedance Measurement of Ex Vivo Rat Lungs at Different Airway Pressures

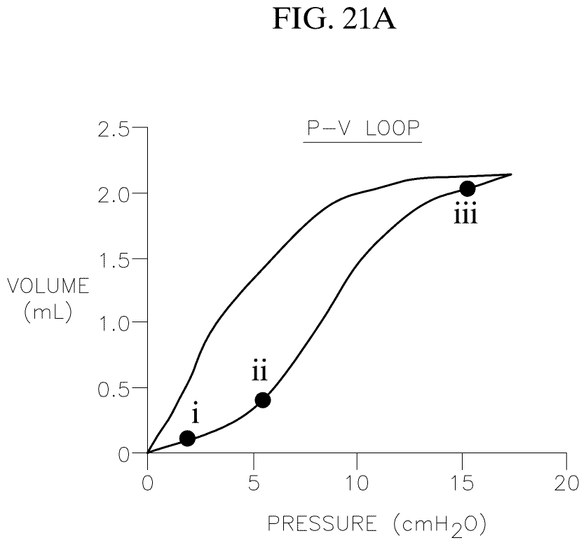

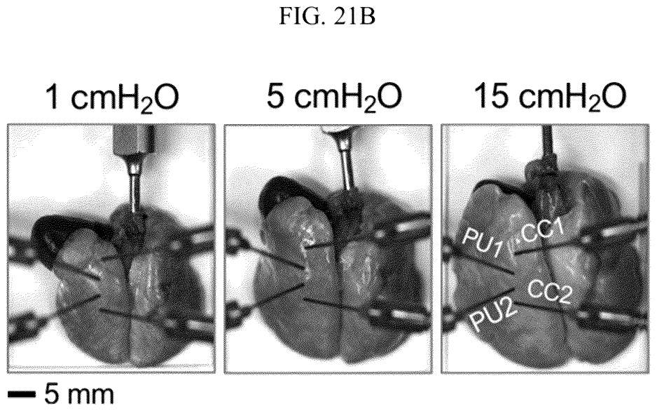

The electrical impedance of the ex vivo lungs at different airway pressure levels was measured (FIG. 21A). The airway pressure was maintained at constant levels (1, 5, 15 cmH2O) without ventilating the lungs to minimize motion-induced measurement error and prevent tissue damage that could result from uncontrolled contact of the measurement electrodes with the lung surface (FIG. 21B). An alternating electrical current (AC) with various frequencies (range: 100 Hz-300 kHz) and a maximum amplitude of 100 μA were injected into lung tissue using two current-carrying electrodes (CC1 and CC2), while two voltage pickup electrodes (PU1 and PU2) measured the voltage at the measurement site (FIG. 21B). The measured electrical impedance (Z) of the lungs at different airway pressure levels followed the same pattern with the change in frequency. Furthermore, the impedance increased with an increase in the lung's airway pressure at all frequencies (FIGS. 27C, 27D). As the lung pressure increases, the internal lung volume increases, causing stretching of the lung tissue at the alveoli septa, which narrows the conductive pathways for electrons/ions, consequently increasing electrical impedance values. Notably, the presence of more air in the alveolar airspace (acting as an electrical insulator) and stretched lung tissue (thinner alveolar septa) creates fewer conducting electrical pathways between measurement electrodes.

### Example 3: Bioimpedance Measurement of Acid-Injured Pathological Lung Tissues

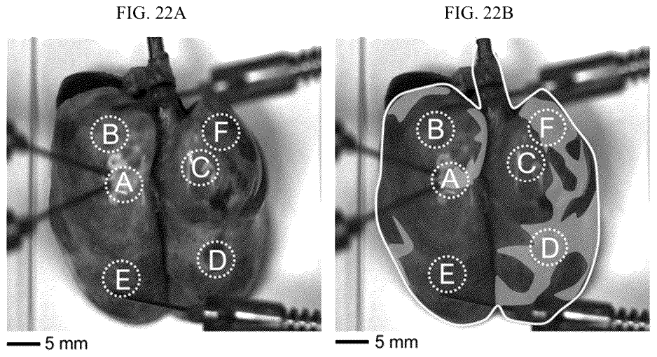

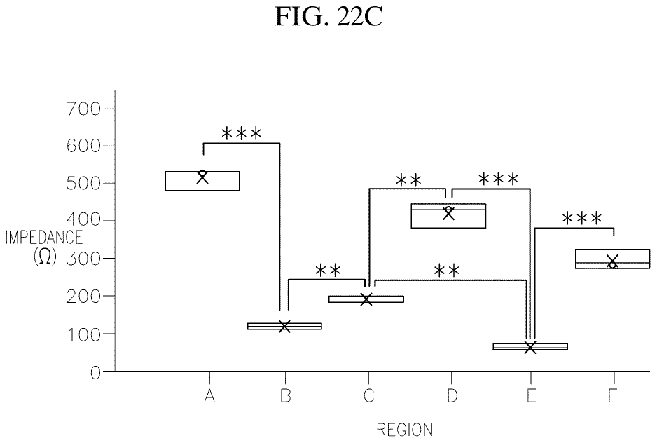

It was further investigated whether the electrical impedance of the lung could be altered by creating an acid-induced lung injury model in the lungs (FIGS. 27E-27I). Intratracheal instillation of hydrochloric acid (HCL) solution, a commonly used solution to model gastric aspiration-induced lung injury, was achieved to create the pathological condition (FIG. 27E). To this end, different volumes (1 and 5 mL) of HCl solution (0.1 N, pH level: 1.5) were intratracheally instilled into the lungs via a 10 mL syringe connected to the trachea cannula (FIG. 27F). After each HCL instillation, 0.5 ml of air was injected through tubing to ensure that HCl had reached the distal parts of the lungs. Histological images (Hematoxylin & Eosin, DAPI) of the acid-injured lungs (5 mL HCL) confirmed excessive accumulation of liquid and debris in the alveolar airspace and increased thickness of the alveolar septa (FIG. 27I). Additionally, quantification of histological images showed decreased alveolar airspace. The results indicated that HCL-induced injury led to a decrease in the electrical impedance of the lung (FIG. 27G, 27H). The reduction of electrical impedance in injured lungs was connected to the replacement of air with liquid in the airspace and an increase in the alveolar septa thickness due to tissue dissociation, which collectively provide more conductive pathways for electron/ion mobility. Further, the electrical impedance of different regions of acid-injured lungs treated with 2 mL HCL solution (0.1 N, pH level: 1.5) was measured (FIGS. 22A-22C). Random regions (A, B, C, D, E, and F) were selected for impedance measurements (FIGS. 22A, 22B). The injured regions were identified by the change in the color of lung tissue, being primarily affected by the HCL solution. Collectively, the results showed lower impedance values of acid-injured regions (B, C, E) compared to non-injured regions (A, D, F) in the lungs (FIGS. 22A-22C), highlighting the functionality of the bioimpedance measurements in detecting the acid-injured pathological tissues. FIG. 22A shows injury achieved through intratracheal instillation of 2 mL of 0.1 N HCL into the lungs. The injured regions appear nightingale-brown due to tissue dissociation in the presence of HCL. FIG. 22B showed indications of non-injured and injured tissue. Random regions (A, B, C, D, E, and F) were selected for impedance measurements. FIG. 22C The shows the lower impedance values of acid-injured regions compared to non-injured regions in the lungs.

### Example 4: Bioimpedance Measurements of Human-Derived Healthy and Fibrosis Lung Sections

Idiopathic pulmonary fibrosis (IPF) is a chronic and progressive lung disease with an unknown cause, characterized by progressive tissue scarring at the lower respiratory system, irreversible loss of lung function due to fibrosis, and median survival of 3-5 years after diagnosis. In IPF, excessive fibrillar collagen and elastin deposition occurs in the ECM of the interstitial lung tissue during ECM remodeling, which triggers the events causing lung fibrosis. Early diagnosis of IPF can improve treatment outcomes in patients. However, due to the similarity of IPF symptoms with other pulmonary conditions and the complexity of the disease, there are still no reliable diagnostic or prognostic biomarkers for IPF.

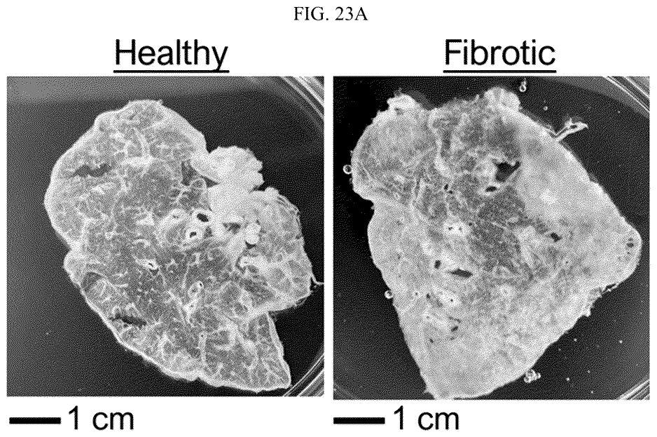

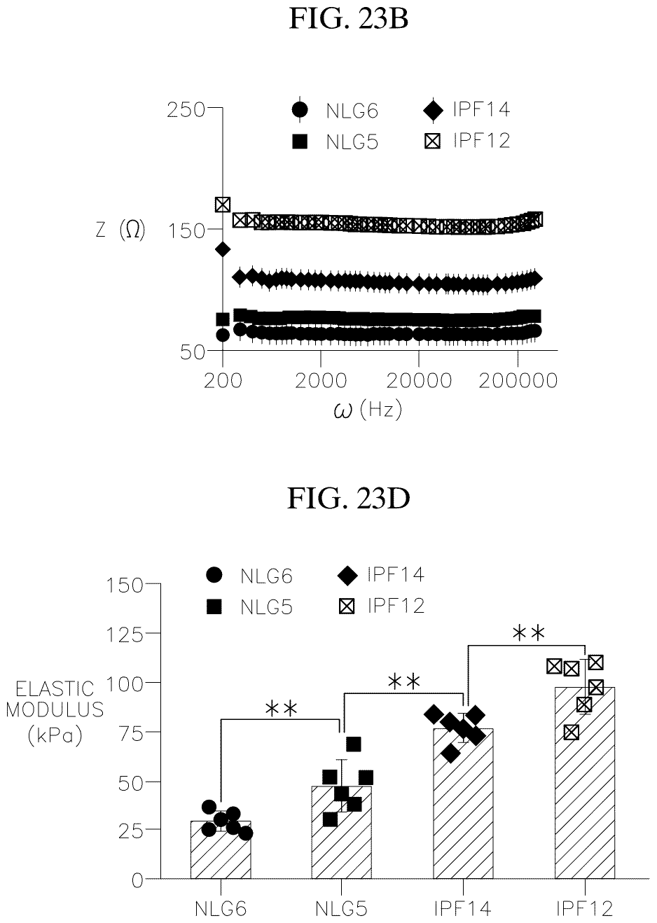

Since the resistance (R) reflects the ECM structure and composition, and the reactance (X) is correlated with the dynamic performance of the cellular membrane, the electrical impedance of human-derived healthy and fibrotic lung sections was assessed to investigate the role of tissue fibrosis on the lung electrical impedance (FIG. 23A). The photographs show tissue sections (thickness: 2 mm) from healthy and fibrotic lungs. The lungs were procured from the New York Organ Donor Network (NYODN) under a protocol approved by the Institutional Review Board at Columbia University. The lungs were from two healthy donors with no history, diagnosis, or evidence of smoking and lung diseases (labeled by NLG5 and NLG6) and two patients with end-stage lungs diagnosed as idiopathic pulmonary fibrosis (IPF) lungs by transplant surgeons (labeled by IPF12 and IPF14). The lung tissues were sectioned into 2 mm-thick slices using a vibratome device. The sections were kept in 2×PBS for 15 min and decellularized with a series of decellularization CHAPS solutions (8 mM) at 4° C. on an orbital shake. To measure the impedance of the lung sections, the sections were cut in rectangle shapes (2 cm height×1 cm width×2 mm thickness), and the measurements were conducted at room temperature.

The electrical impedance values showed higher electrical impedance in fibrotic tissues compared to healthy lungs at all frequencies (FIG. 23B). Electrical Impedance was measured while varying the frequency of alterative current from 30 Hz to 300 kHz. The results show the electrical impedance increases when the lung tissue becomes fibrotic. The increased electrical impedance of fibrotic lungs was connected to the increased tissue stiffness of these lungs due to the deposition of collagen and elastin in the fibrotic tissue. Further, the mechanical properties of healthy and fibrotic tissues were analyzed with a tensile testing platform (FIG. 23C). FIG. 23C is a photograph of a tensile testing setup for measuring the stiffness of healthy and fibrotic human lung sections. The elastic modulus (E) measurement at low strain showed higher stiffness in fibrotic tissues compared to healthy lung tissues, which may be correlated with the augmented deposition of collagen and elastin in fibrotic tissues (FIG. 23D).

### Example 5: Bioimpedance Measurements of Ex Vivo Rat Trachea

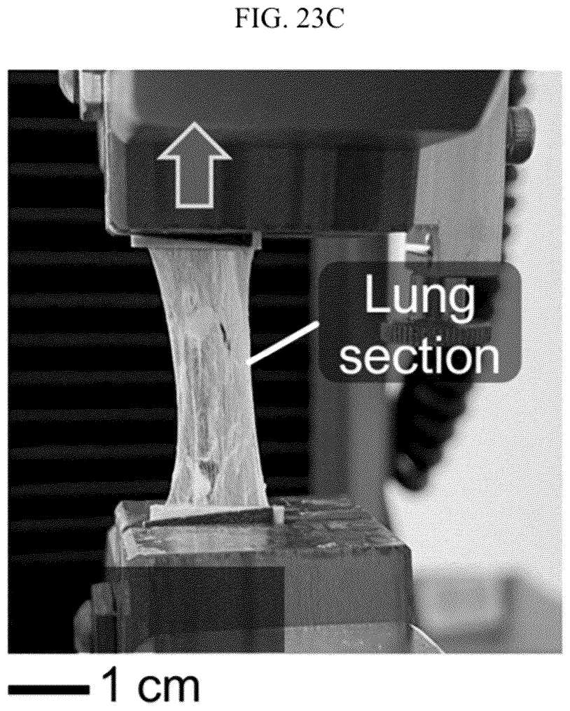

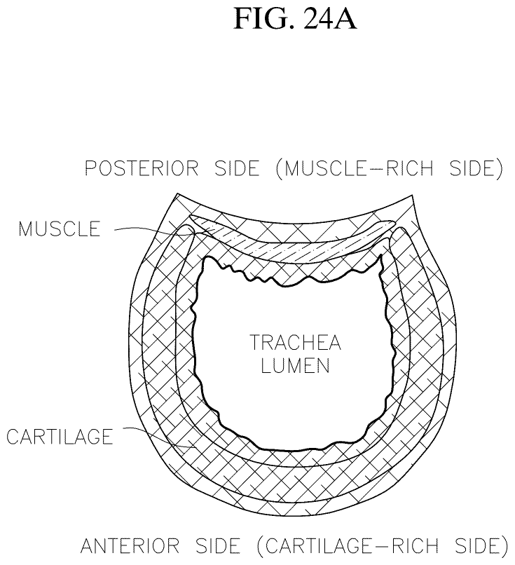

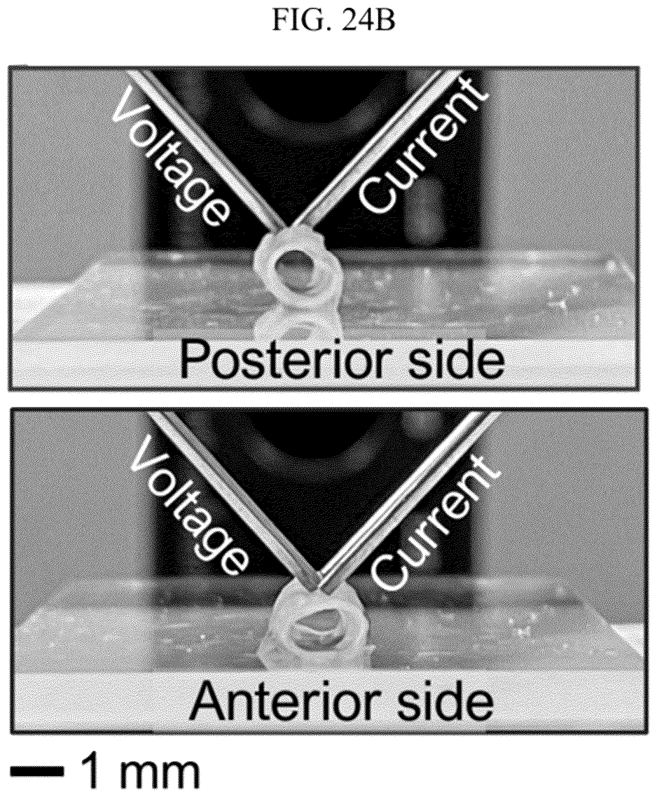

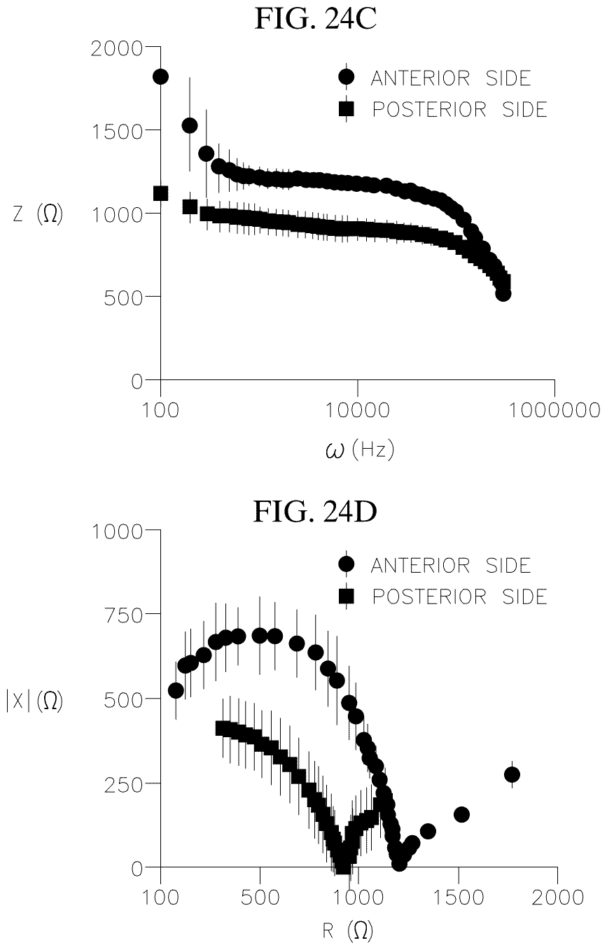

In a previous study, the change in bioimpedance of airway tissue after creating a localized injury on the epithelial layer of the airway was investigated. It was found that disruption of airway epithelium leads to reduced electrical impedance measured at the airway lumen due to local disruption of epithelial cells, the main contributors to the hindrance of alternate current flow at low frequencies. Given that the trachea is composed of a C-shaped cartilaginous ring (anterior part) and a muscle tissue through which the trachea is connected to the esophagus (exterior part), the question of whether this composition variation across the trachea cross section could be detected by measuring electrical impedance with the present measurement setup (FIG. 24A) was investigated. FIG. 24A is a schematic showing a cross section of the trachea. The posterior side of the trachea is attached to the esophagus and is mainly composed of muscle tissue. The anterior side of the trachea is predominantly composed of cartilaginous C-shaped rings.

To this end, the impedance on the trachea's exterior surface was measured by positioning the electrodes on muscle (anterior) and cartilaginous (posterior) parts of the trachea (FIG. 24B). The photographs of FIG. 24B show the measurement electrodes in contact with trachea tissue. Alternative current with varying frequency of 100 Hz to 300 kHz was injected into anterior and posterior sides of the trachea, and the voltage as measured at both sides. The measurements showed that the overall bioimpedance (Z) of the anterior part was higher than the posterior part in most frequencies (FIGS. 24C, 24D). FIG. 24C shows impedance measured at different frequencies. The results show that cartilage-rich parts have higher electrical impedance compared to the muscle-rich parts. FIG. 24D shows Cole-Cole plots for the anterior and posterior sides of the trachea (Z: impedance. R: resistance, X: reactance, ω: frequency). The results are consistent with the findings in the literature, where cartilage tissue has less electrical conductivity (higher electrical resistivity) than muscle tissue.

### Example 6: Bioimpedance Measurements of Decellularized Ex Vivo Rat Lung and Trachea

In tissue engineering and regenerative medicine, “decellularization” is a process by which the cellular components are removed from the tissue to create biological scaffolds. The purpose of decellularization is to remove all the immunogenic components from the tissue to prevent immunoreaction with newly seeded cells. Ideally, the decellularized scaffolds can be seeded with patient-sourced stem/progenitor cells and produce a transplantable, bioengineered tissue.

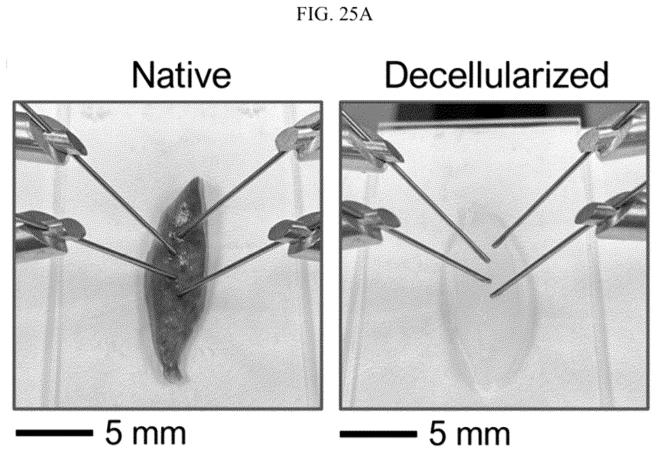

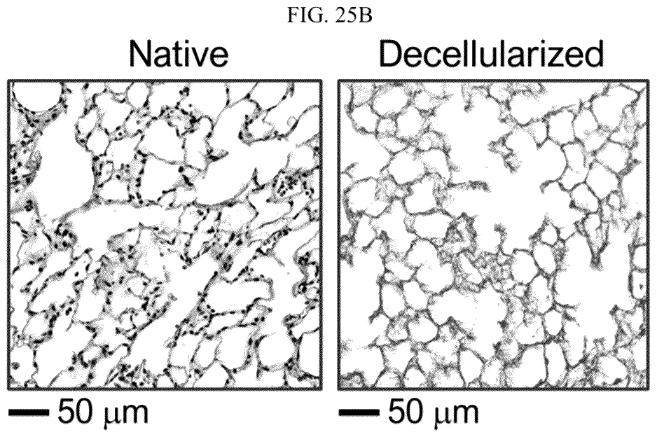

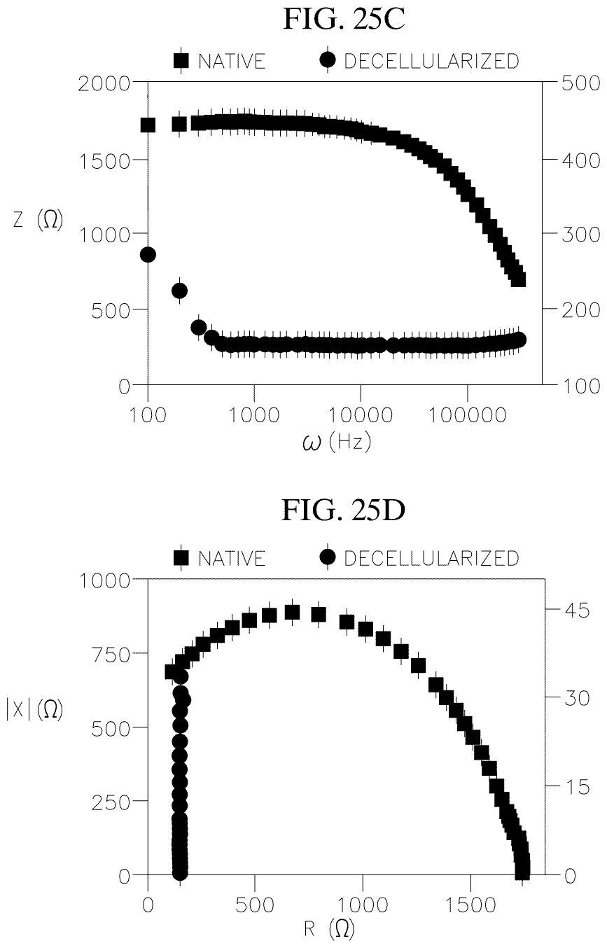

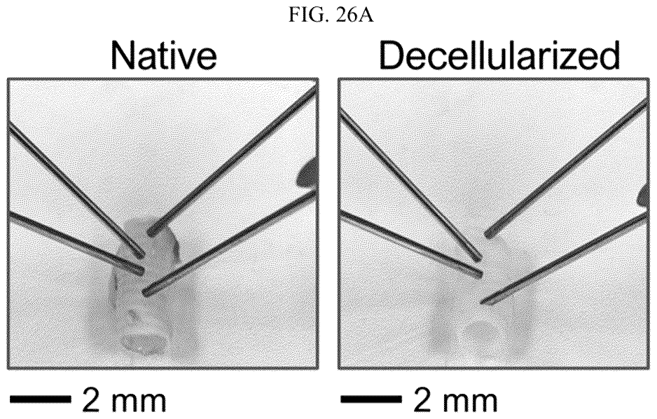

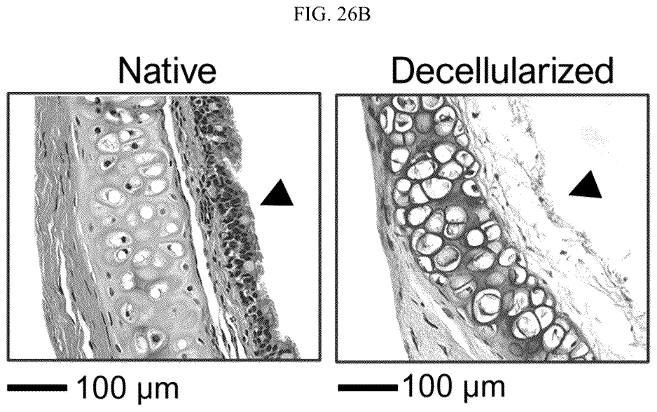

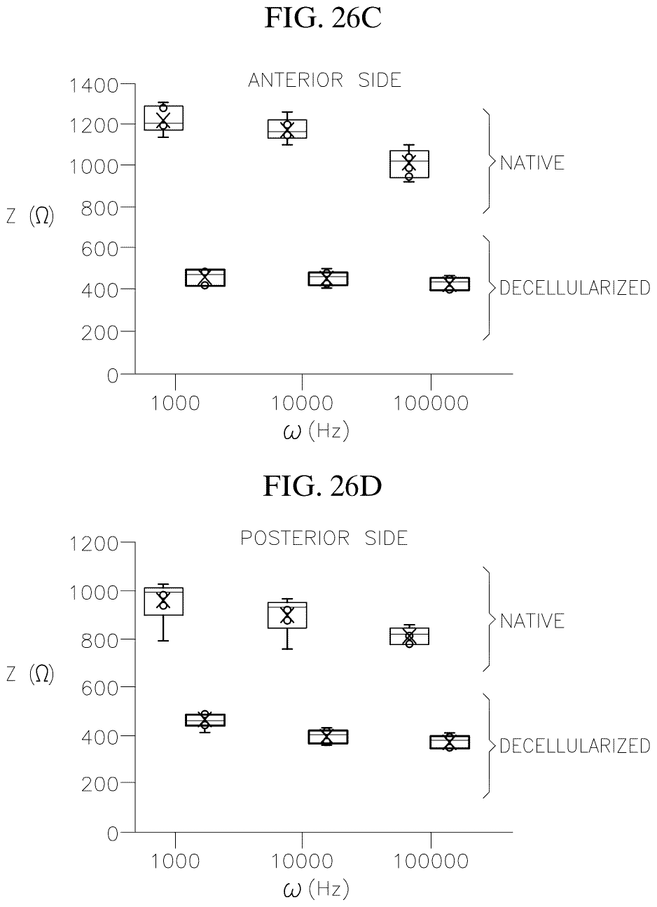

Since all cellular components are removed from the tissue during decellularization, and the cell membrane significantly contributes to the overall electrical impedance of the tissue, the influence of decellularization on the electrical impedance of rat lungs and trachea (FIGS. 25A-25D and 26A-26D) was investigated. To create decellularized ex vivo lungs, the lung tissue sections (thickness: 2-3 mm) were treated with a series of decellularization agents (Triton X-100 and sodium dodecyl sulfate (SDS)). The impedance was then measured for both native and decellularized lung sections (FIG. 25A). The photograph of FIG. 25A shows the positions of electrodes on the surface of native and decellularized lung sections. Alternating current with varying frequency of 100 Hz-300 kHz was injected into lung tissues and voltage was measured. The histological images (Hematoxylin & Eosin, DAPI) of native and decellularized lung tissues showed the absence of cellular content and nuclear materials in decellularized lung sections (FIG. 25B). The decellularized lung sections show the removal of all cell nuclei (>99%) compared to the native lung sections. Notably, the impedance measurement of decellularized lungs showed significantly lower impedance compared to native lungs (FIGS. 25C and 25D). FIG. 25C shows impedance measured at different frequencies for native and decellularized lung tissues. The left and right y-axes represent the impedance values of native and decellularized lung sections, respectively. Results show decreased impedance in decellularized lungs. FIG. 25D shows Cole-Cole plots for native and decellularized tracheas. Left and right y-axes represent the reactance values of native and decellularized lung sections, respectively (Z: impedance. R: resistance, X: reactance. ω: frequency).

Further, the decellularized tracheas were prepared by treating the trachea with a series of Triton X-100 solutions (FIGS. 26A-26D). The electrical impedance was measured at the interior and posterior sides of the native and decellularized tracheas (FIG. 26A). The photograph of FIG. 26A shows the positions of electrodes on the surface of native and decellularized tracheas. Alternating current with varying frequency of 100 Hz-300 kHz was injected into the tracheas, and voltage was measured. The Hematoxylin & Eosin and DAPI staining confirmed the removal of the cellular components in decellularized tracheas. The histological images of FIG. 26B show native and decellularized tracheas. The decellularized tracheas show reduction of cell nuclei (>99%) compared to the native trachea. The electrical impedance of native and decellularized tracheas was measured at three different frequencies (i.e., 1000, 10000, and 100000 Hz) on the anterior and posterior sides of the trachea. Similar to lung tissue measurements, the electrical impedance of decellularized tracheas was lower than that of native intact tracheas for both anterior (FIG. 26C) and posterior (FIG. 26D) sides of the trachea. In the plot of FIG. 26C, impedance was measured at three distinct frequencies for the anterior side (i.e., cartilage-rich part) of native and decellularized tracheas. In the plot of FIG. 26D, impedance was measured at three distinct frequencies for the posterior side (i.e., muscle-rich part) of native and decellularized tracheas. In all frequencies, the decellularized tracheas showed lower impedance values compared to the native tracheas (Z: impedance. ω: frequency. Decell: decellularized. Arrowhead indicates trachea lumen).

### Example 7: Bioimpedance Measurements of Fibrotic and Acutely Injured Lung Tissues

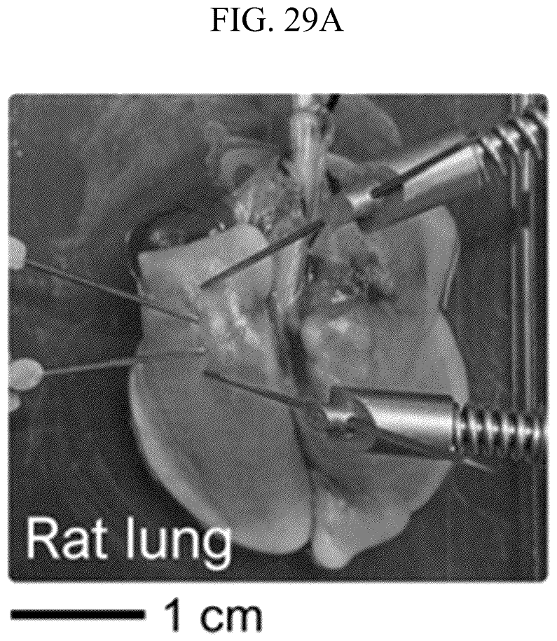

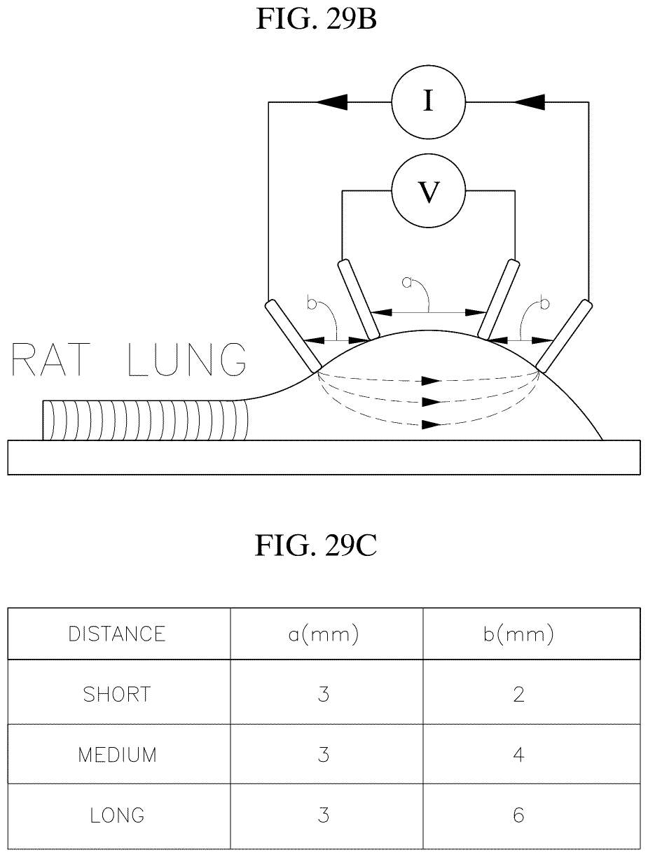

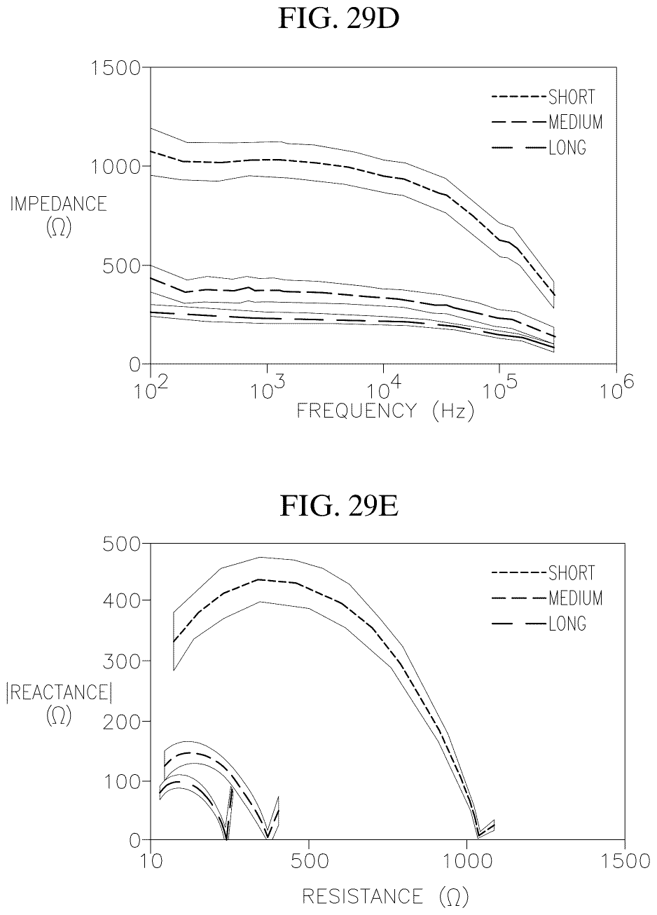

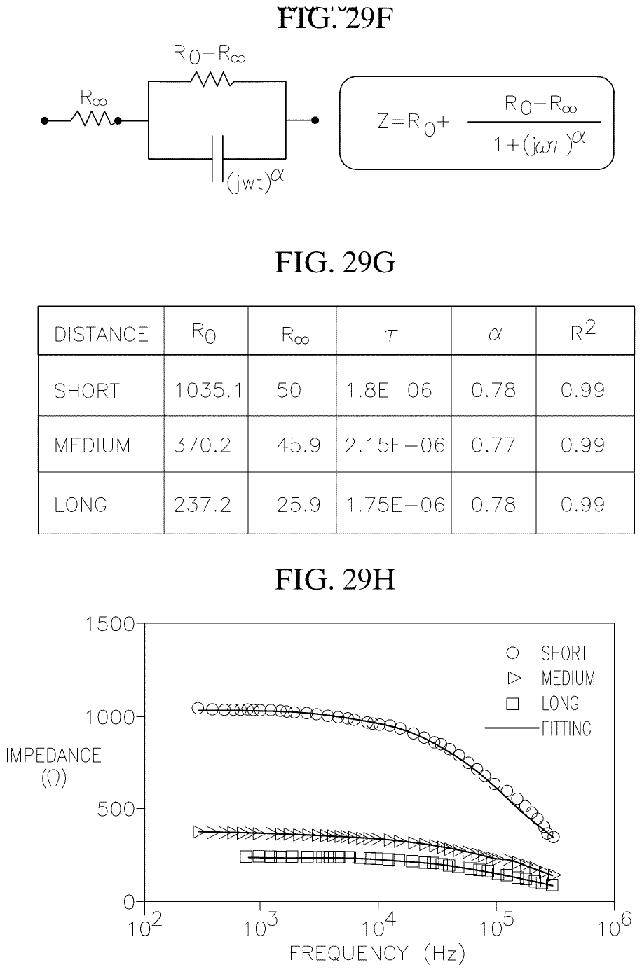

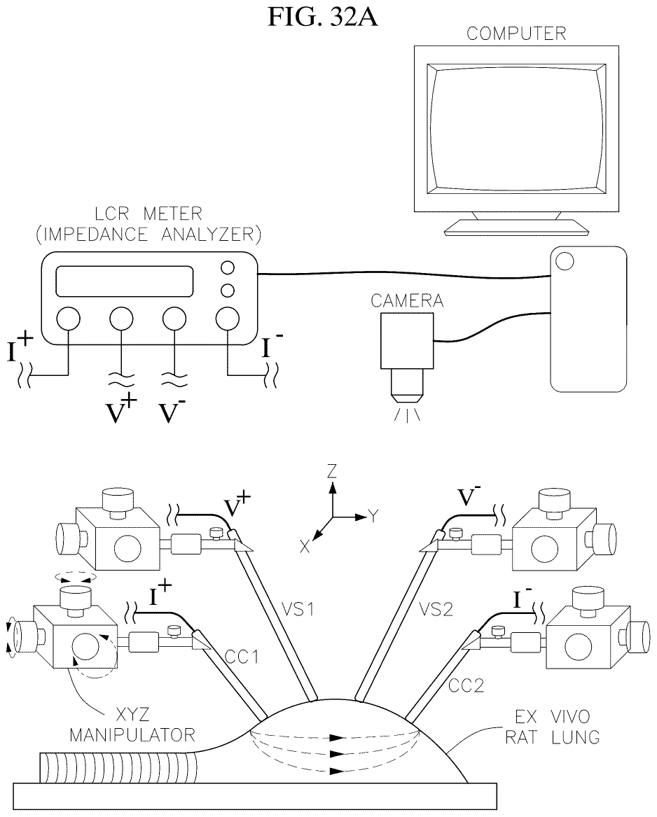

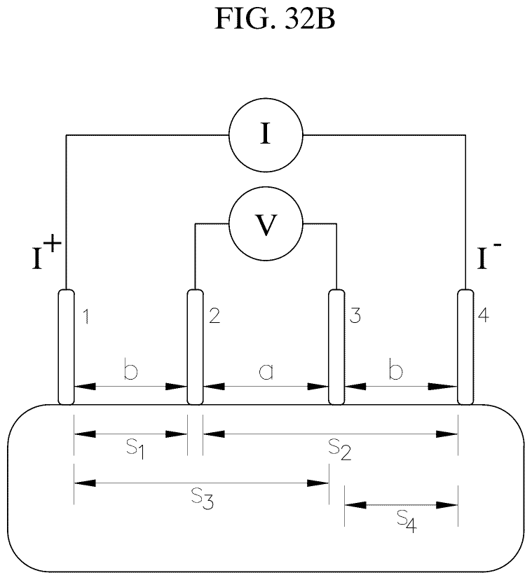

The role of the electrode probe spacing on the bioimpedance characterization of the rat lung parenchyma tissue was initially investigated (FIGS. 29A-29H). The electrode probes were arranged in a linear fashion on the pleural surface of the lung. The distance between the voltage-sensing probes (i.e., inner probes, a) was kept constant at 3% mm, and the spacing between the voltage-sensing and current-carrying probes (i.e., outer probes, b) was varied (2% mm: short, 4% mm: medium, and 6 mm: long) using XYZ micromanipulators (FIGS. 29A-29H; FIGS. 32A-32B). During measurements, the alveolar pressure (P_{ALV}) of the lungs was maintained at 2% cmH2O, and no mechanical ventilation applied. Each probe is attached to an XYZ micro-manipulator (see FIG. 32A) that enables precise arrangement on the tissue surface. The arrangement of electrodes is monitored by a custom-built imaging system. A schematic of the linear arrangement of probes with unequal spacing on the 3D tissue and different spacing parameters is seen FIG. 32B. LCR: inductance-capacitance-resistance. I: current. V: voltage. CC: current-carrying electrode probe. VS: voltage-sensing electrode probe.

FIG. 29A is a photograph showing the position of electrode probes on the pleura surface of ex vivo rat lungs FIG. 29B is a schematic showing the spacing variables (i.e., a and b) in linear arrangement of the probes: a: distance between voltage-sensing probes; b: distance between current-carrying electrodes and voltage-sensing probes. FIG. 29C is a table presenting different spacings between the probes: a was kept constant while b was changed. FIG. 29D shows bioimpedance of lung parenchyma tissue measured at different alternating current (AC) with frequency values (range: 0.1 to 300 kHz). The bioimpedance level was decreased by increasing b. FIG. 29E is Cole-Cole plots at different probe spacings. FIG. 29F shows a Cole-Cole circuit and equation. FIG. 29G calculated parameters of a Cole-Cole bioimpedance model. Z: bioimpedance. ω: angular frequency. R₀: resistance at zero frequency (i.e., DC). R∞: resistance at the infinite frequency. a: a dimensionless number (0<a<1). r: characteristic time constant. FIG. 29H shows measured bioimpedance data and the best fit indicated by a solid line for the extraction of Cole-Cole parameters. Data are presented as mean+standard deviation.

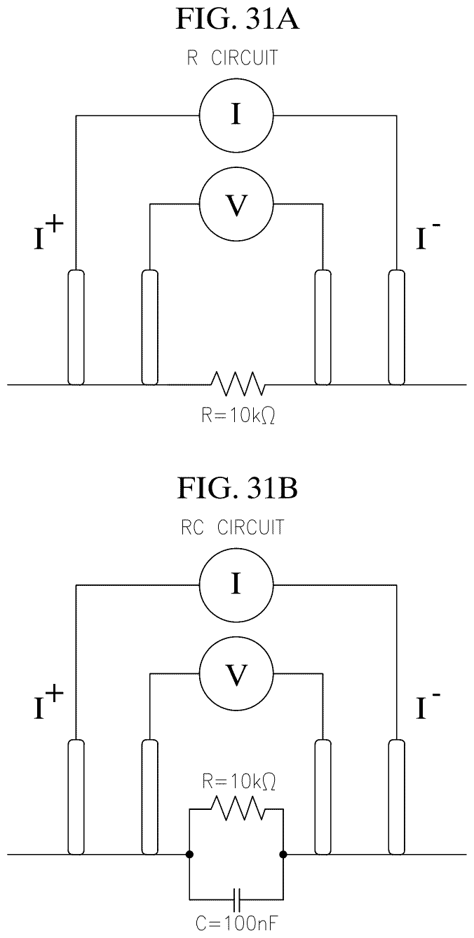

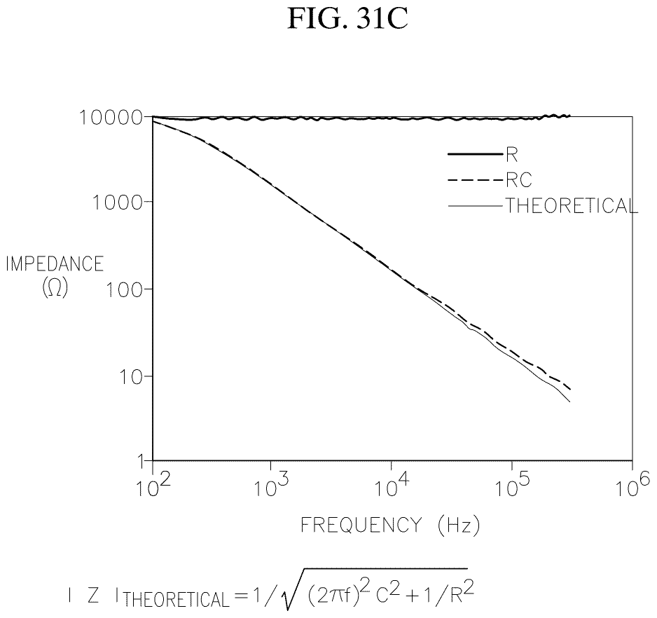

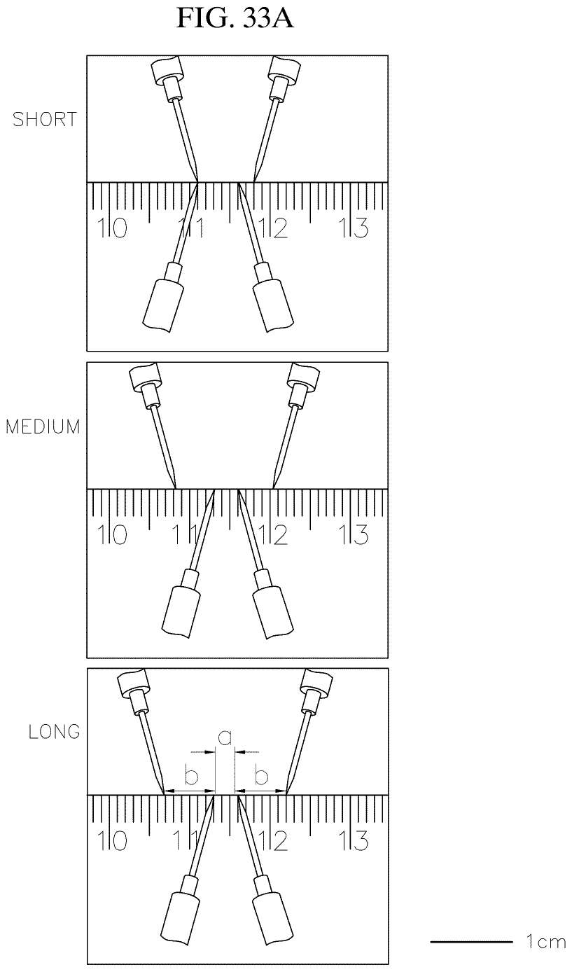

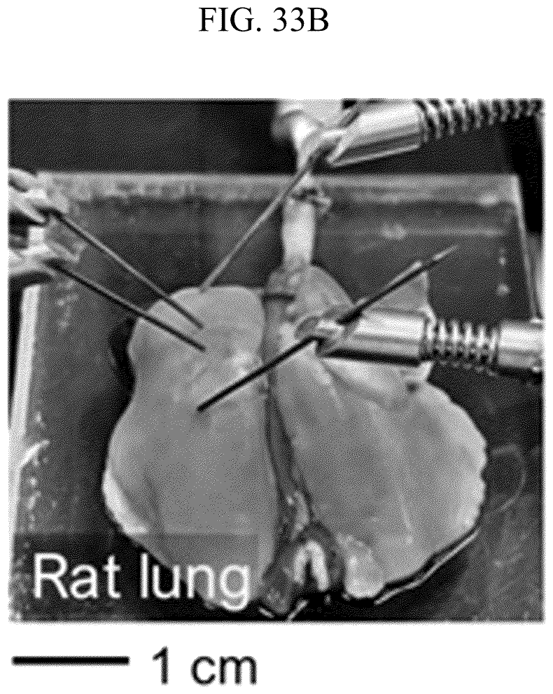

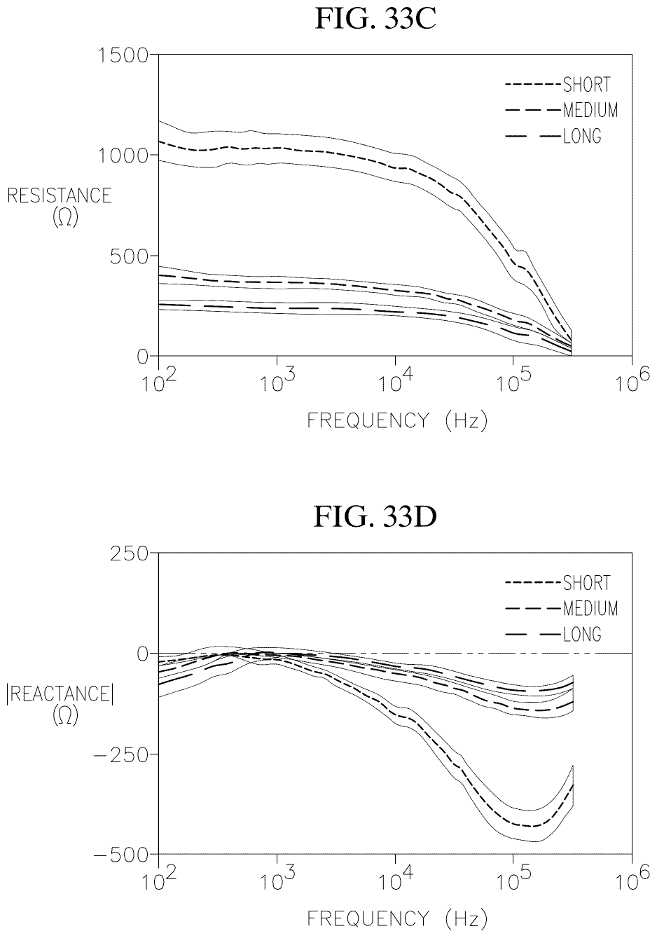

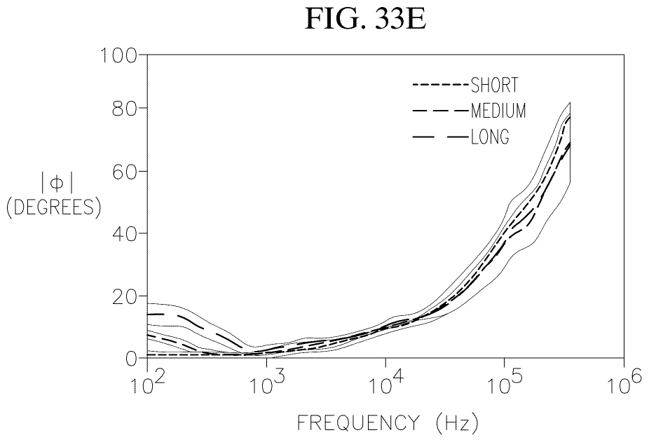

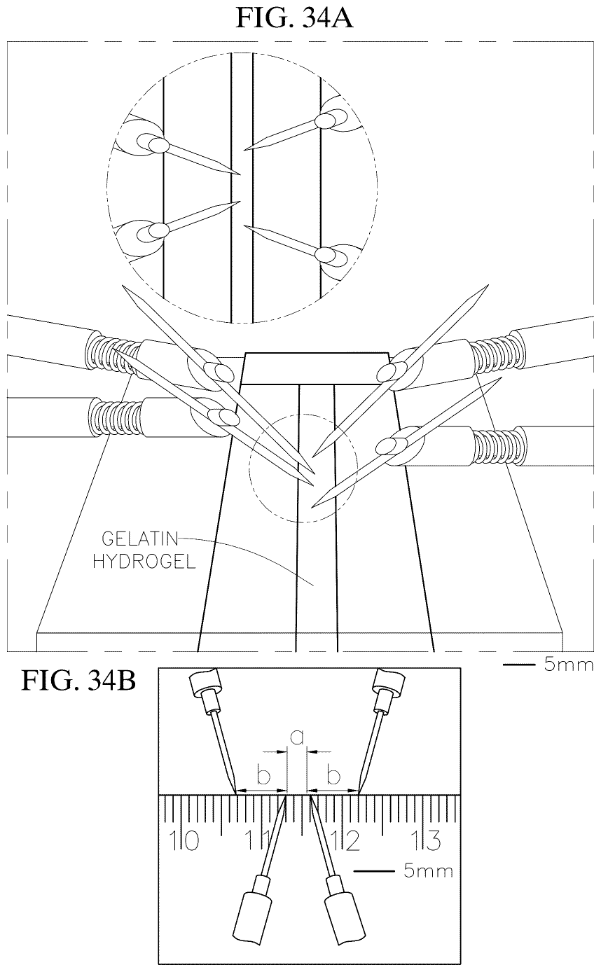

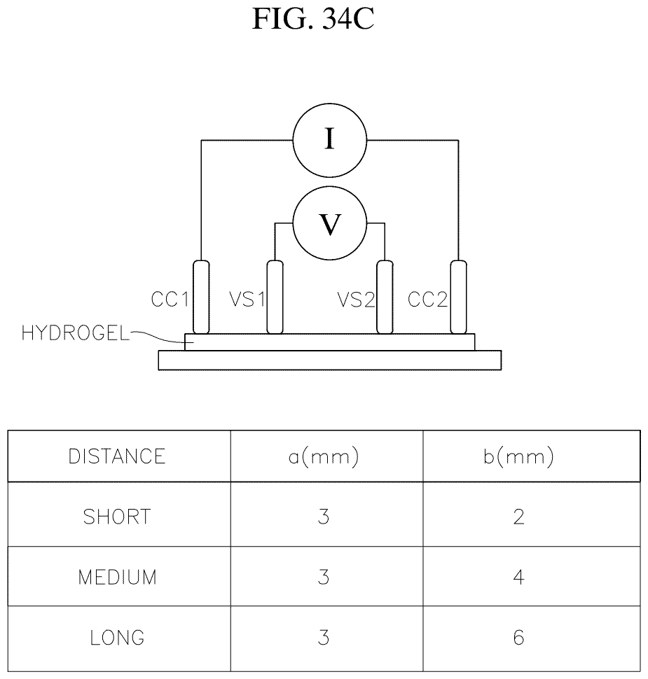

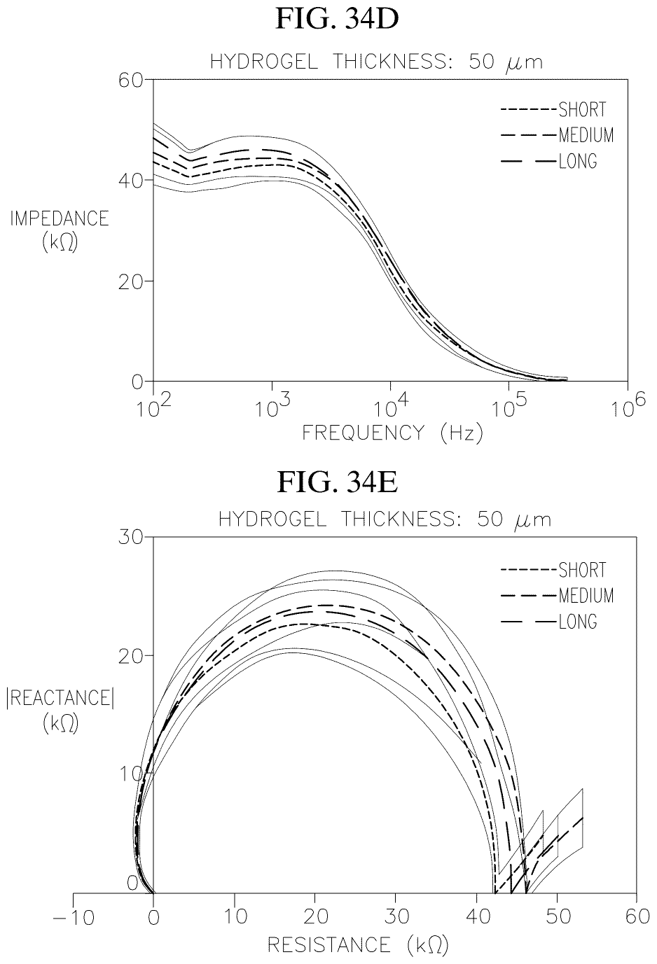

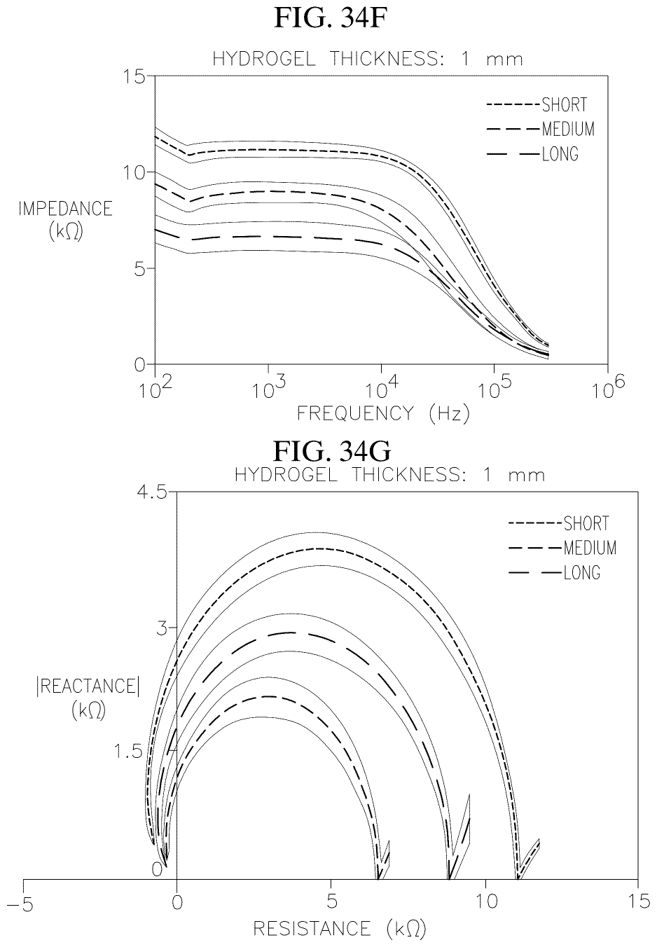

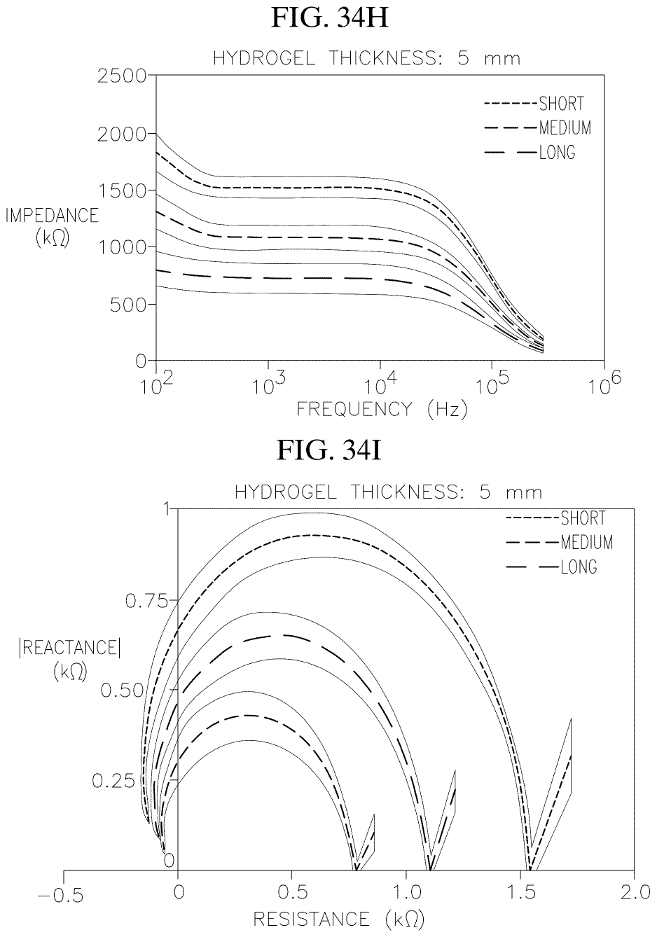

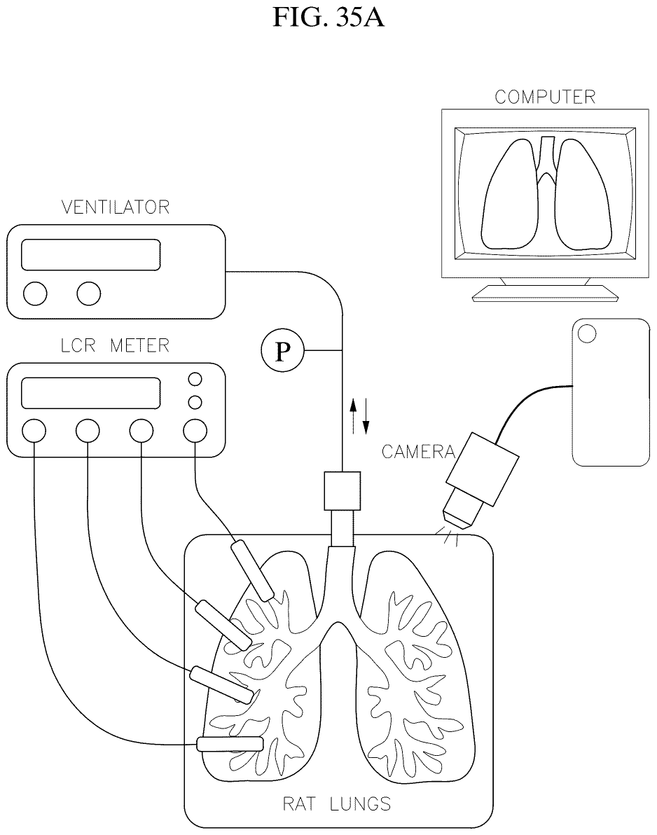

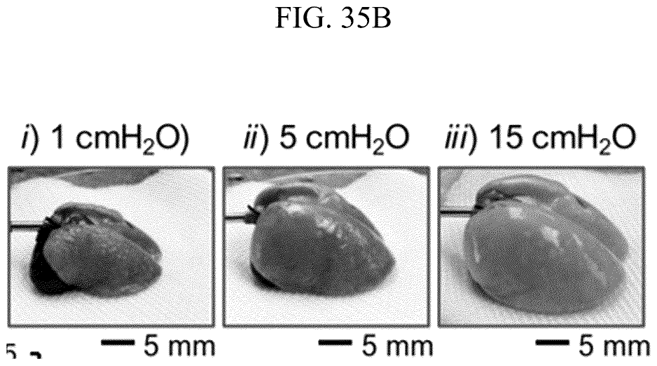

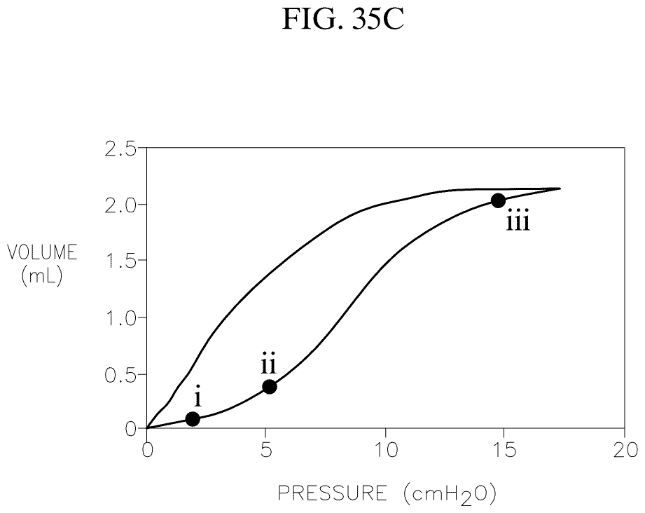

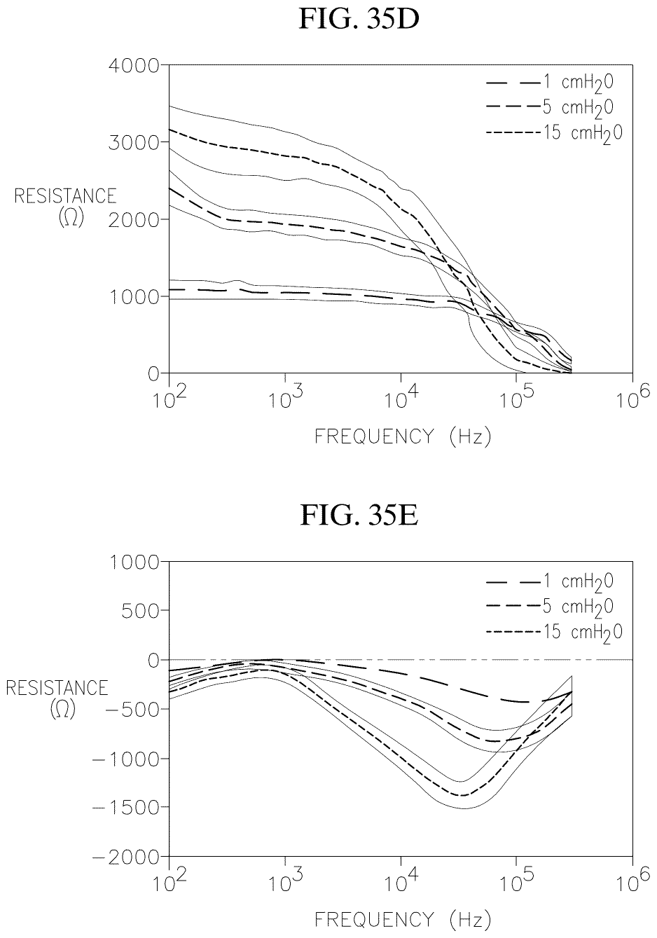

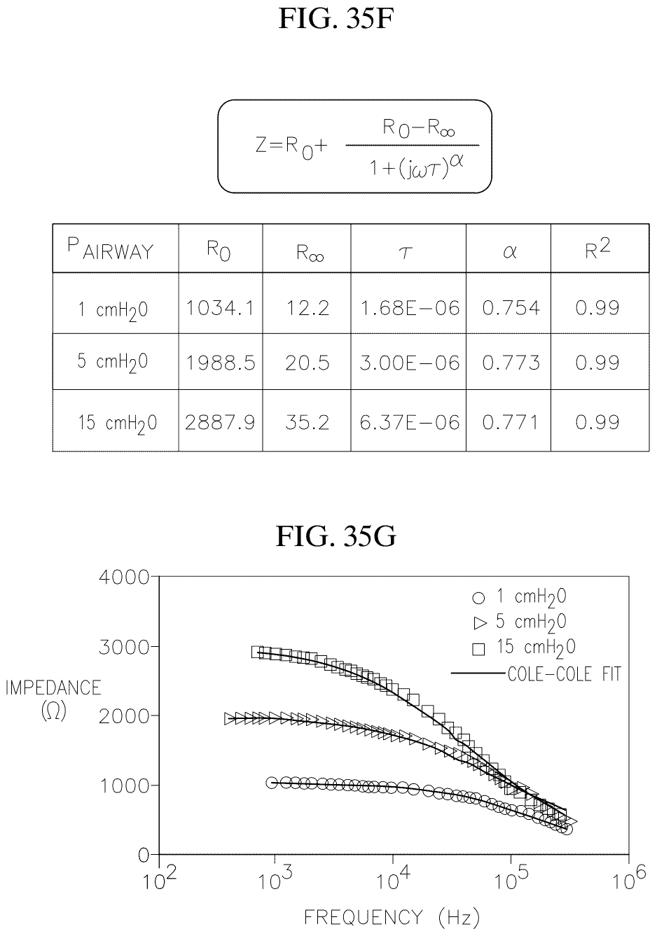

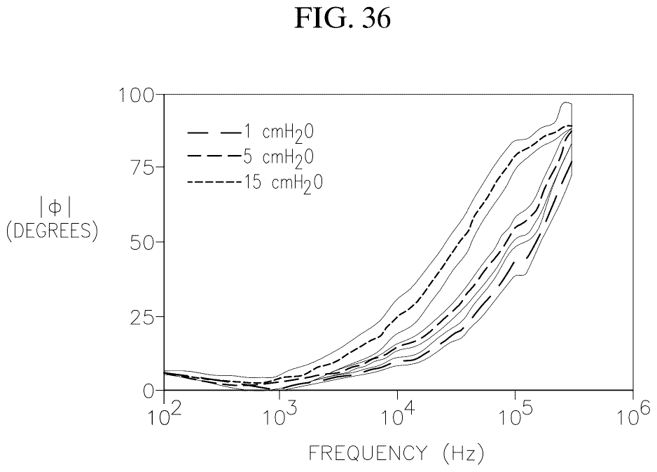

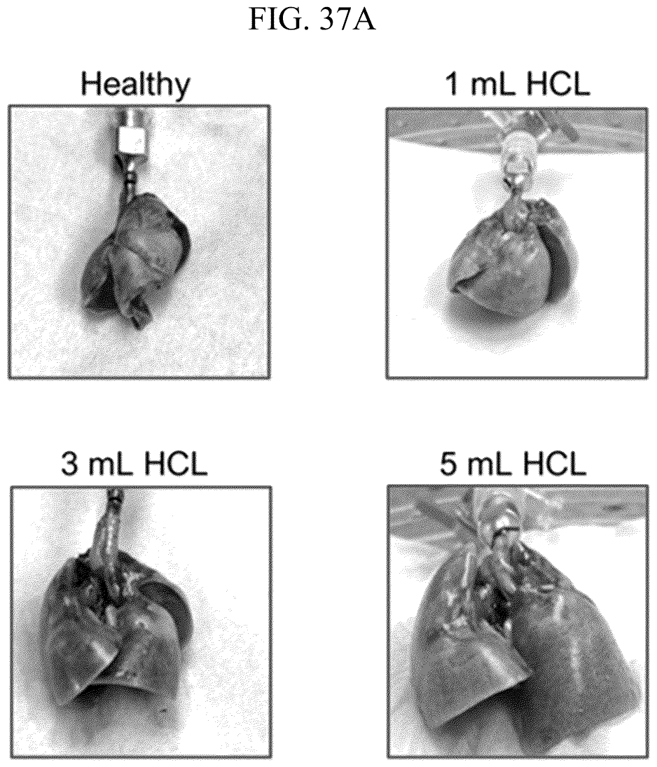

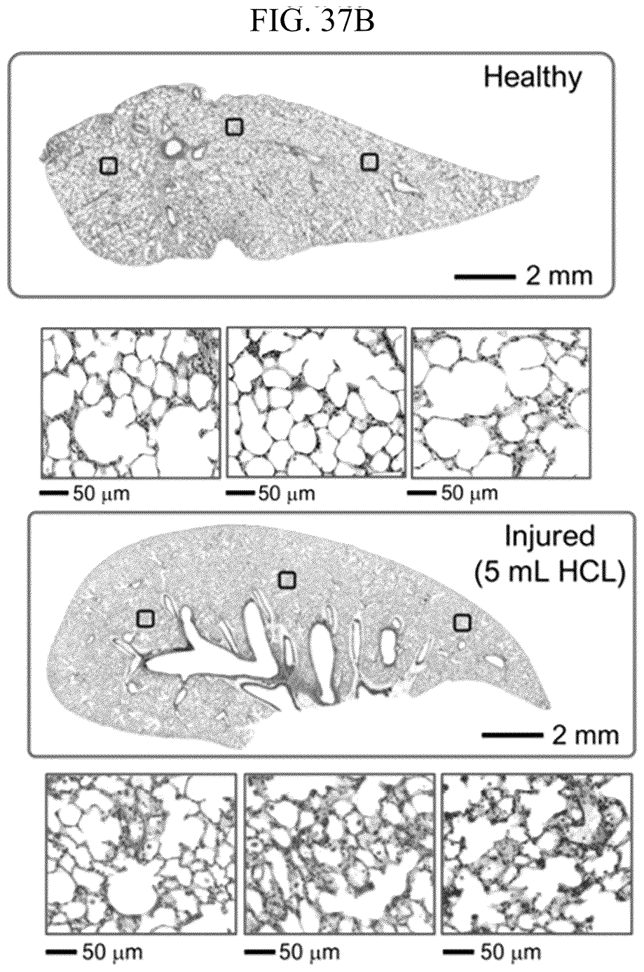

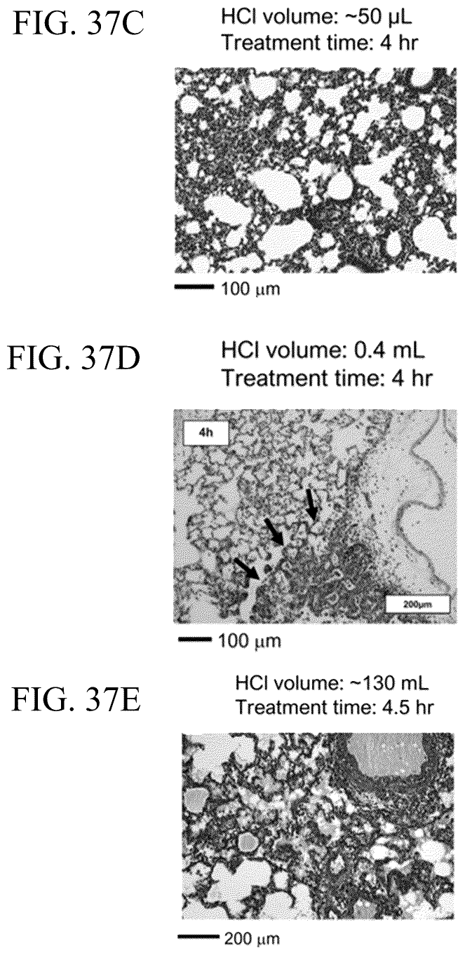

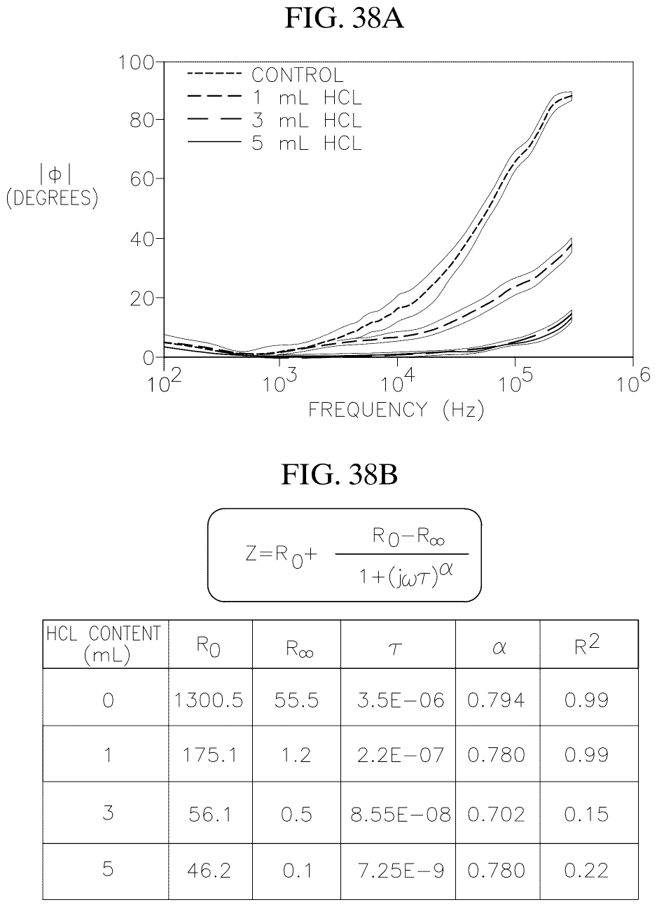

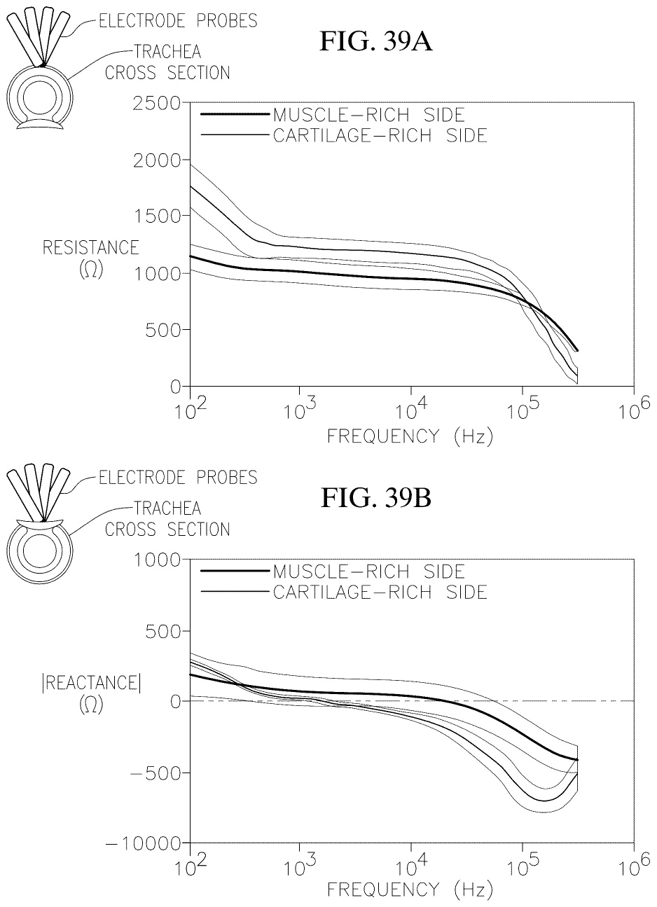

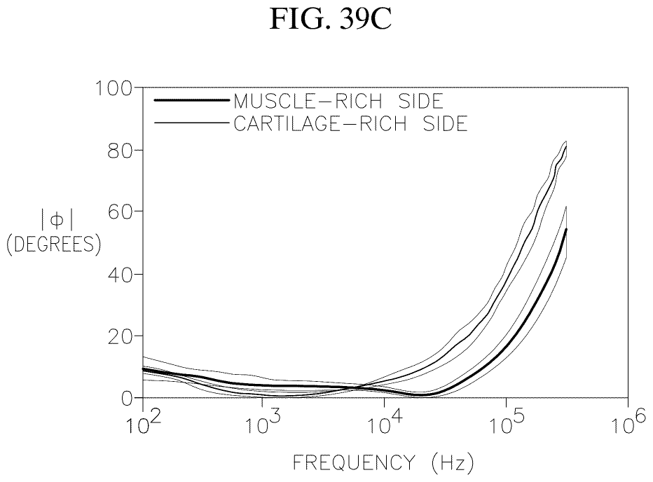

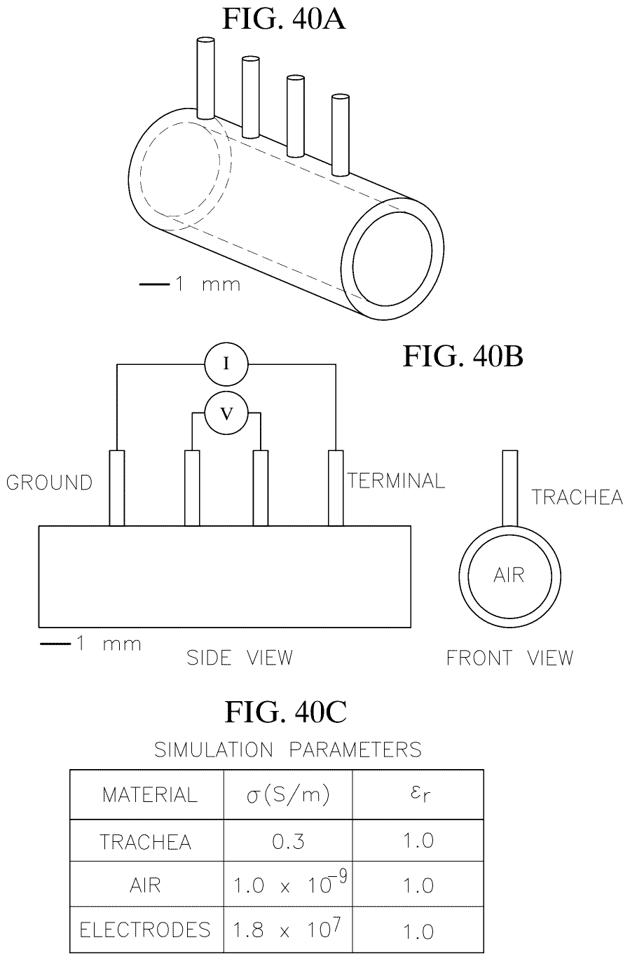

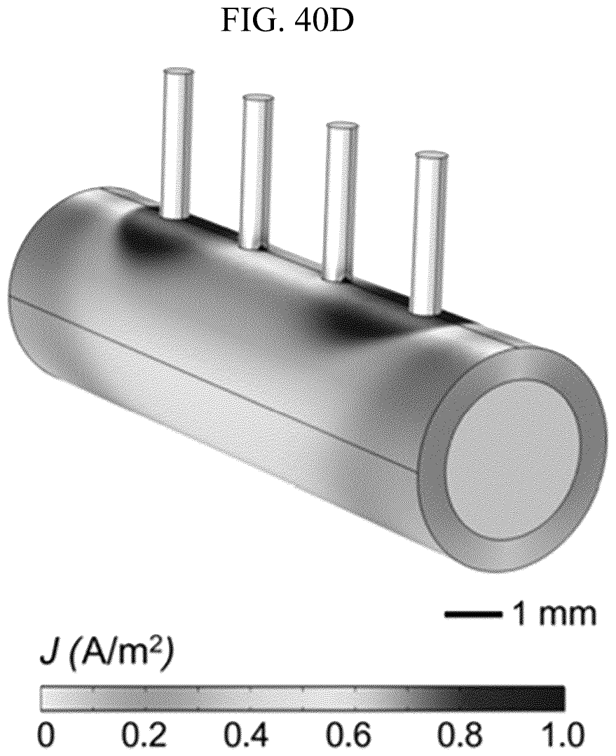

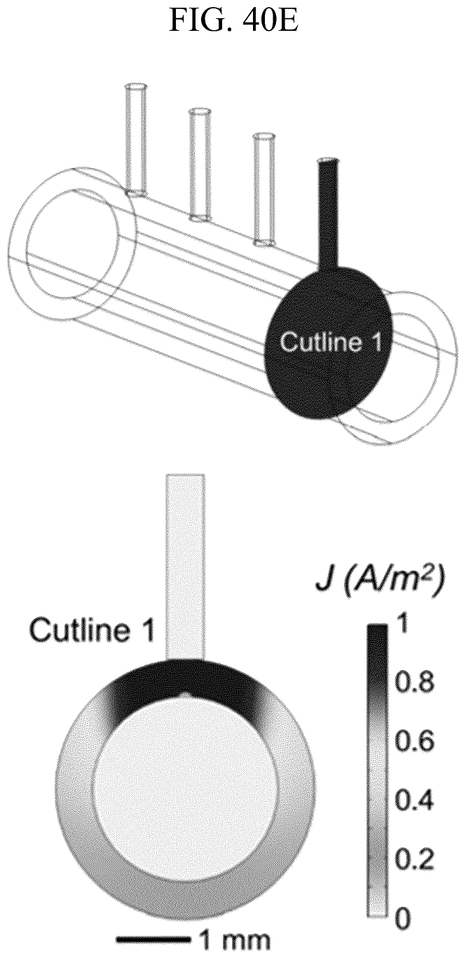

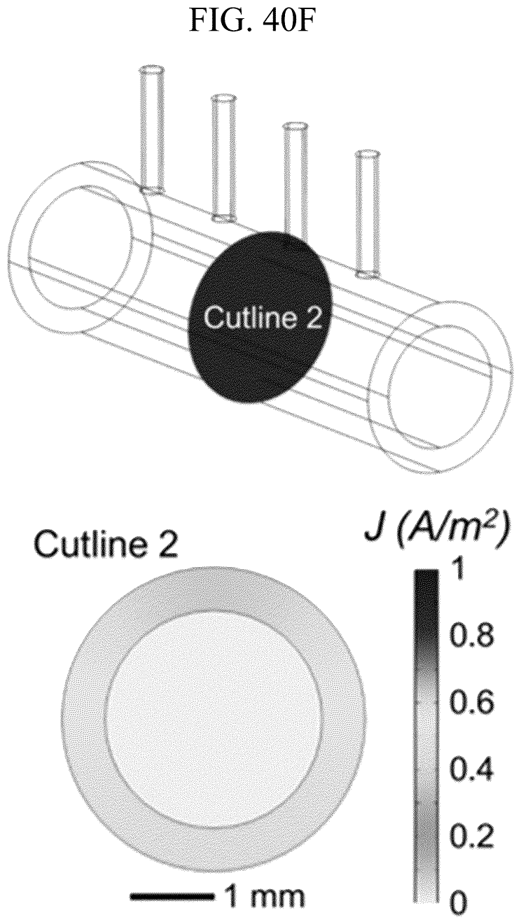

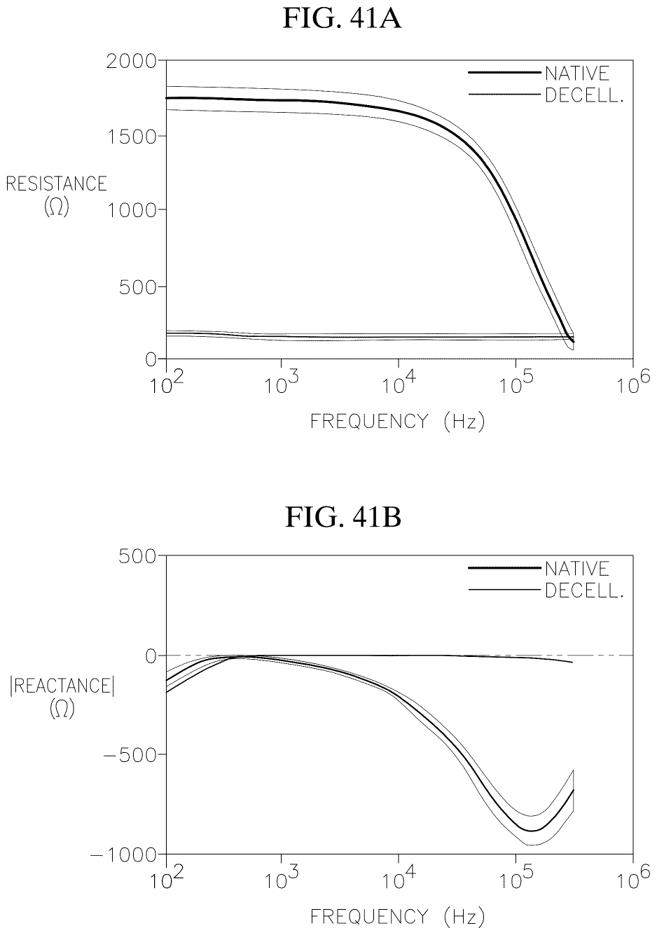

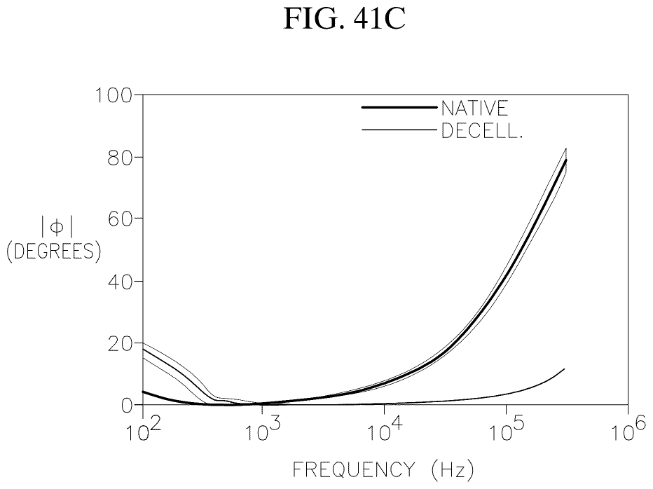

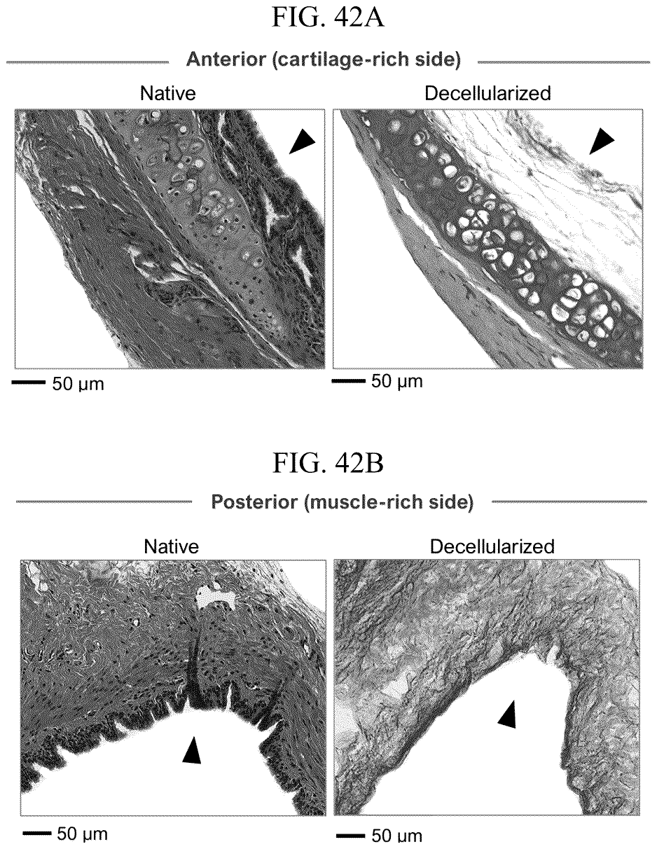

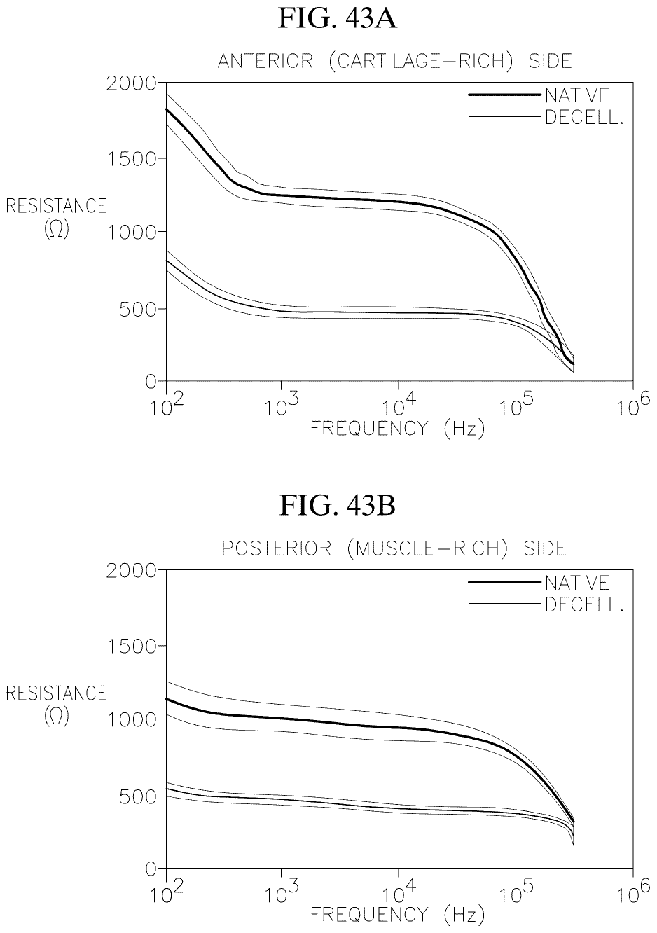

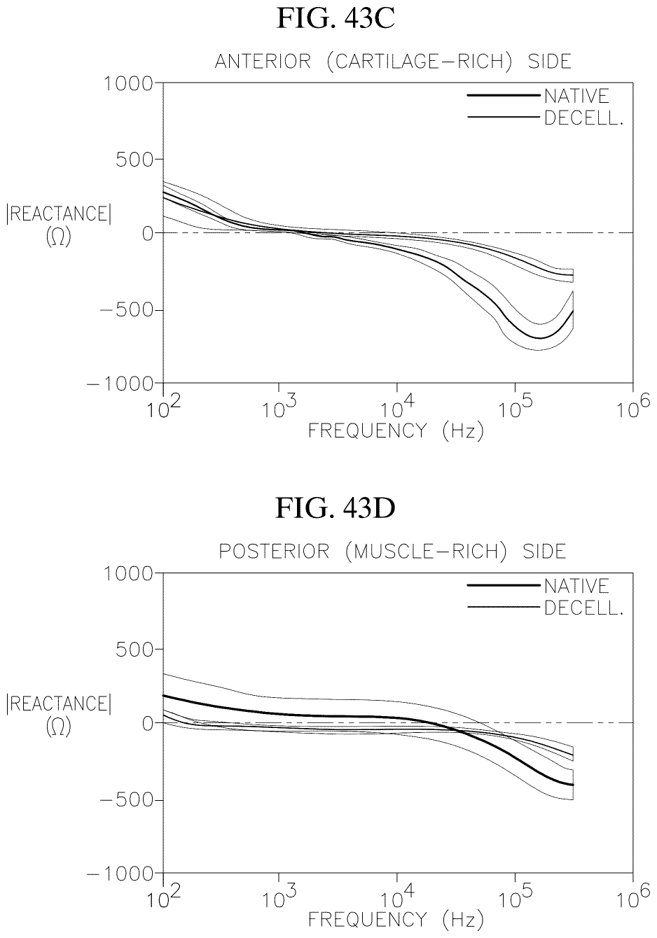

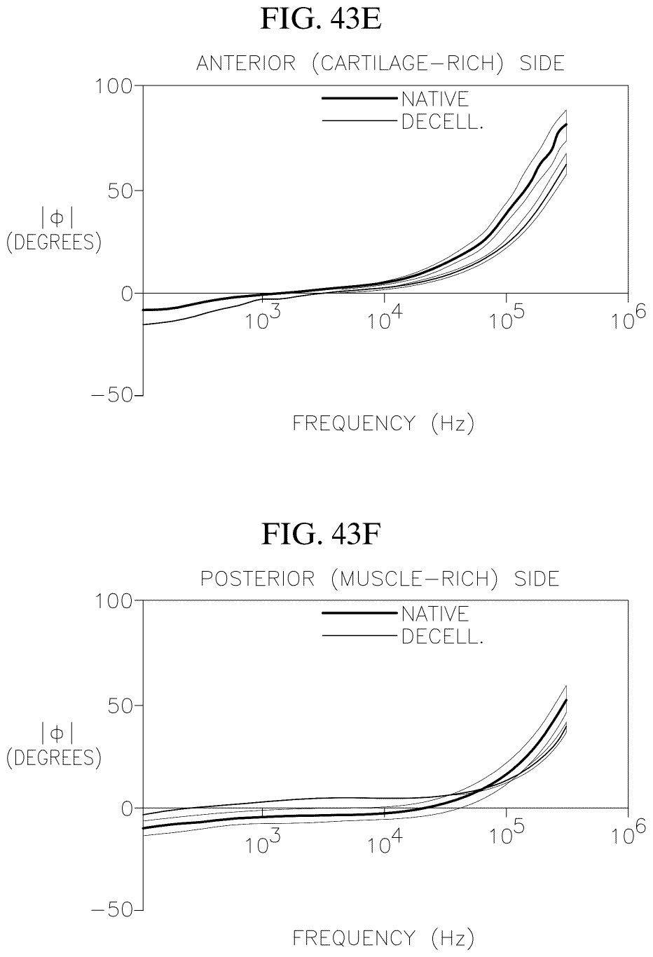

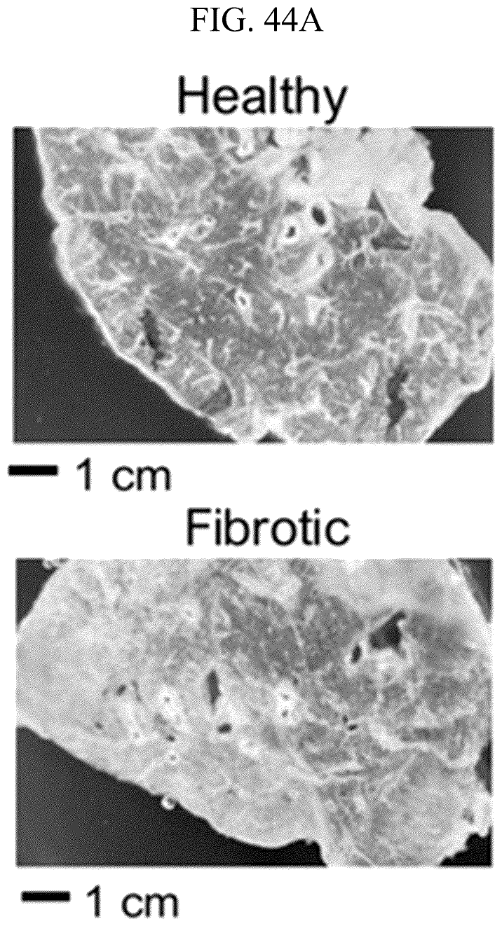

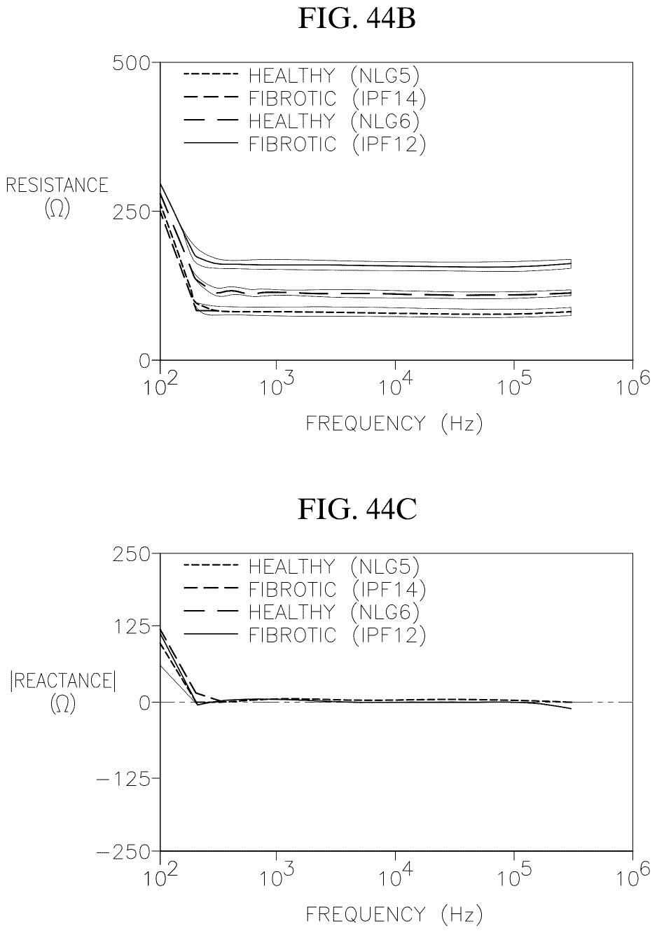

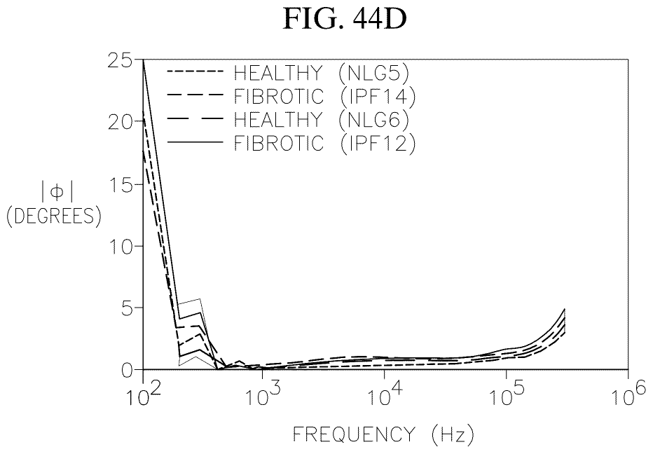

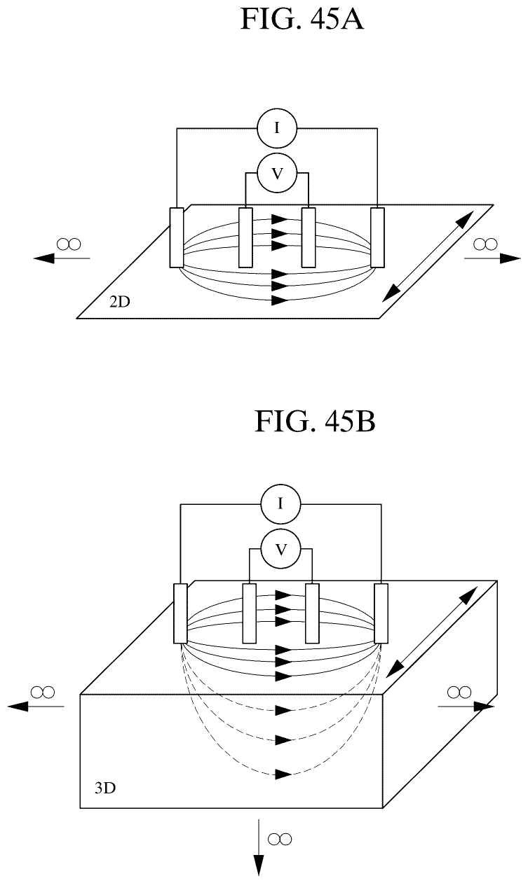

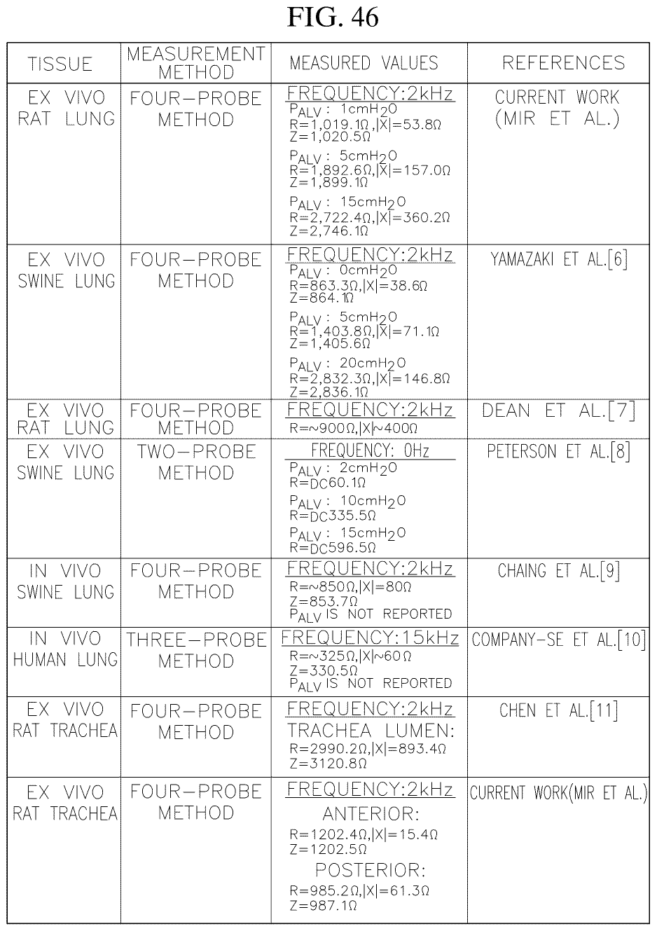

FIGS. 31-46 detail additional data, including RC circuit measurement using Keysight E4980AL-032 LCR meter (FIGS. 31A-31C), schematic of the bioimpedance measurement setup (FIGS. 32A-32B), the effect of electrode probes spacing on lung tissue bioimpedance (FIGS. 33A-33E), bio-impedance measurements of gelatin-based tissue phantoms (FIG. 34A-34I), the effect of airway pressure on lung tissue bioimpedance (FIGS. 35A-35G), phase angle assessment of lung parenchyma tissue at different lung pressure (P_{ALV}) levels (FIG. 36), histological assessment of acid-injured lungs (FIGS. 37A-37E), phase angle plot and Cole-Cole parameters for acid-injured lungs (FIGS. 38A-38B), bioimpedance measurement of rat trachea (FIGS. 39A-39C), Computer-simulated electrical current flow in the tracheal tissue (FIGS. 40A-40F), bioimpedance measurement of decellularized lung sections (FIGS. 41A-41C), H&E images of decellularized tracheas (FIGS. 42A-42B), bio-impedance measurement of decellularized rat trachea (FIGS. 43A-43F), bio-impedance measurement of native and fibrotic human-derived lung sections (FIGS. 44A-44D), schematic of current flow propagation in 2D and 3D materials (FIGS. 45A-45B), and electrical resistance, reactance, and bio-impedance of lung tissues reported in the literature (FIG. 46).

The lung tissue bioimpedance showed a consistent frequency-dependent behavior at different probe spacings throughout the frequency spectrum ranging from 0.1 to 300 kHz (FIG. 29D). The magnitude of bioimpedance decreased by enlarging the spacing b for all frequencies. This behavior is primarily due to decrease in electric field (E) and the voltage drop (ΔV) between the electrode probes, which results in a decrease in overall resistance and bioimpedance. The reduction in bioimpedance due to an increase in the spacing b was more pronounced at frequencies below 100 kHz. Notably, this probe spacing dependency of bioimpedance aligns with the theoretical models developed for electrical resistance of three-dimensional (3D) materials, where the resistance of 3D materials is inversely proportional to the spacing between the probes.

Moreover, the measured resistance and reactance values showed contrasting trends to the low- and high-frequency current at different probe spacings (FIGS. 33A-33E). FIG. 33A is a schematic showing linear arrangements of the probes (a-b-a: 2-3-2, short; 4-3-4, medium; 6-3-6 long, unit: mm). FIG. 33B is a photograph presenting the arrangement of the probes in a linear fashion on the lung pleural surface. Plots for resistance (33C), reactance (33D), and phase angle (33E) of lung parenchyma across the frequency range of 0.1 to 300 kHz.

At low frequencies, the difference between resistance values was more significant. This behavior may be due to the high resistance of the cell membrane to low-frequency electrical current, which results in the electrical current passing only in the extracellular matrix surrounding the cells. As the frequency of the current increased, the difference between resistance values decreased, while the difference between reactance values increased. This observation may be due to the penetration of high-frequency currents into the cell membrane, causing the capacitance characteristic of the membrane to become more pronounced. Notably, as high-frequency current passes through the membrane of the cells, the current field uniformly spreads across the tissue, leading to a substantial decrease in both tissue resistance and overall bioimpedance. The magnitude of resistance was higher than the magnitude of reactance (|R|<|X|) at frequencies below 100 kHz, indicating that the bioimpedance of lung tissue is primarily determined by its resistance (i.e., resistive response) in this frequency range. Conversely, at frequencies above 100 kHz, the lung tissue showed a capacitive response (|X|<|R|). Notably, the calculated phase angles of tissues at different probe spacings showed no significant difference at all frequencies (FIGS. 33A-33E).

To further confirm the role of probe spacing on the bioimpedance of 3D tissues using the above measurement setup, gelatin-based tissue phantoms (concentration: 5% w/v) were prepared with different thickness levels (50 μm, 1 mm, and 5 mm) and their bioimpedance values measured (FIGS. 45A-45I). The results showed that the bioimpedance of the 50 μm thick phantom sheet did not change significantly across all frequencies, while the bioimpedance of tissue phantoms with thicknesses of 1 mm and 5 mm decreased as the spacing between probes increased. The change in bioimpedance due to probe spacing was greater in 5 mm thick phantoms compared to 1 mm thick phantoms. This observation suggests that as the thickness of the hydrogel sample decreases, the impact of probe spacing on the bioimpedance magnitude diminishes.

FIG. 34A is a schematic showing the positioning of the probes on hydrogel surface. FIG. 34B is a schematic and FIG. 34C a table showing probes' linear arrangements (2-3-2: short; 4-3-4: medium; 6-3-6 long; unit: mm). Also shown are plots of bioimpedance (FIG. 34D), Cole-Cole plots (FIG. 34E) for hydrogel with 50 pm thickness, a bioimpedance plot (FIG. 34F) and Cole-Cole plots (FIG. 34G) for phantoms with 1 mm thickness, and a bioimpedance plot (FIG. 34H) and Cole-Cole plots (FIG. 34I) of phantoms with 5 mm thickness. CC: Current-carrying electrode probe. VS: Voltage-sensing electrode probe

The Cole-Cole model describes the electrical impedance of biological tissue as a combination of two resistors (R1=R∞ and R2=R₀−R∞) and a capacitor or constant phase element (CPE, (iωτ)a) (FIG. 29F). The Cole-Cole model was investigated as to whether it can accurately describe the bio-impedance of the lung tissue in the frequency range between 0.1 to 300 kHz (FIGS. 29G-29H). The fitting results showed a high agreement between the measured bioimpedance and the theoretical model, indicating a depressed semicircle characteristic of lung parenchyma tissue bio-impedance (FIG. 29G). Moreover, the Cole-Cole parameters for best fittings were determined for different spacings between the electrode probes (FIG. 29H). The calculated relaxation time (r) and values of the lung tissue were in agreement with the values reported in the literature. Furthermore, while a remained unchanged, the strength of resistance (i.e., the length of the arc in the Cole-Cole plot; R₀−R∞) decreased when enlarging the spacing b.

Next, the bioimpedance of the native intact rat lungs ex vivo was measured when the lung's internal air pressure was subjected to different target alveolar pressures (P_{ALV}; FIGS. 35A-35G). The alveolar pressure was adjusted to constant levels (1, 5, 15 cmH2O), and the electrical measurements were conducted without ventilating the lungs to prevent tissue damage that could result from uncontrolled contact of the electrode probes with the lung pleural surface. A small animal ventilator was used to achieve the target pressure in the lungs, and pressure and airflow sensors were used to monitor the change of pressure and volume in the lungs (FIGS. 34A-34I). During the measurements, the spacing between the voltage-sensing probes was 3 mm, while the spacing between the voltage-sensing and the current-carrying probes was set to 2 mm.

FIG. 35A is a schematic of the setup for measurement of lung internal pressure (P_{ALV}) and bioimpedance. P: pressure sensor. LCR: inductance-capacitance-resistance. FIGS. 35B(i)-(iii) are photographs of ex vivo rat lungs at different P_{ALV} levels (1, 5, and 15 cmH2O). FIG. 35C is a pressure-volume (PV) curve of rat lungs at a tidal volume of 2.2 mL/kg. FIG. 35D is a plot of resistance, and FIG. 35E is a plot of reactance of lung parenchyma tissue at different P_{ALV} levels across the frequency spectrum (range: 0.1 to 300 kHz). FIG. 35F shows the Cole-Cole equation and Cole-Cole parameters obtained from fitting the equation with bioimpedance measurements. FIG. 35G is a graph illustrating bioimpedance of lung at different P_{ALV} levels and respective Cole-Cole fit plots. The best fitting is indicated by a solid line.

The lung tissue bioimpedance at different P_{ALV} levels showed a frequency-dependent behavior across the frequency range between 0.1 to 300 kHz. The bioimpedance level increased with an increase in the lung's internal pressure at all frequencies. The observed trend was consistent with the results obtained for swine and calf lungs at different P_{ALV}levels. Such P_{ALV} dependency of bioimpedance could be due to the increase in the internal lung volume that stretches the lung tissue at the alveoli septa. Specifically, with an increase in lung aeration level, the lung airspace volume—which acts as an electrical insulator between the measurement probes—increases, leading to an increase in the resistance between the probes (FIGS. 35A-35G). Additionally, with an increase in volume, the alveoli septa tissue becomes thinner, which narrows the conductive pathways for transferring ions and increases the measured bioimpedance. The increase in P_{ALV} led to a rise in the magnitude of reactance and phase angle, particularly at higher frequencies, due to the increased air volume within the lungs (FIGS. 35A-35G and 36). Further, having the experimental bioimpedance values fitted with the Cole-Cole function, R₀−R_{∞} and τ values increased with an increase in P_{ALV} (FIGS. 35A-35G).

It was further investigated whether the bioimpedance of the lung tissue is altered by pathological conditions, i.e., acid aspiration-induced injury, in the lungs. To this end, different volumes of HCl solution (1, 3, and 5 mL; concentration: 0.1 M, pH level: 1), were intratracheally instilled into the lungs (FIGS. 37A-37E). HCl exerts chemical damage to pulmonary airway and alveoli epithelium, triggering the inflammatory response followed by pulmonary edema and disruption of the alveolar-capillary membrane, which are the common hallmarks of gastric acid aspiration injury. Distribution of the HCl solution within the lung respiratory tract was confirmed through the color change in the lung tissue and histological analysis. Specifically, the regions impacted by HCl solution turned to nightingale-brown. Moreover, H&E images of the acid-injured lungs confirmed excessive accumulation of liquid and debris in the alveolar airspace and swelling of the alveolar septa tissue (FIGS. 37A-37E). The quantification of histological images showed decreased alveolar airspace and increased alveolar septa thickness.

FIG. 37A is a series of photographs showing different volumes of HCL solution (concentration: 0.1 N; volume: 1, 2, 3, and 5 mL) that were intratracheally instilled into the rat lungs. Following each instillation, 0.5 mL air was intratracheally injected to ensure the HCL has reached the distal parts of the lung. The color of impacted tissue regions was changed to nightingale-brown following HCL treatment. FIG. 37B is a series of H&E images of healthy and injured lungs. The histological images of the lungs treated by HCL (volume: 5 mL) shows accumulation of fluid and cell debris in alveolar space and increased alveolar septa thickness. Also seen, for comparison purposes, are histological images of acutely injured mouse (FIG. 37C), rat (FIG. 37D), and pig lungs (FIG. 37E) treated with HCl solution (concentration: 0.1 N).

The bioimpedance measurements were conducted in the same lobe (i.e., left lobe) for both native and acid-injured lungs. The distances were set to 3 mm between the voltage-sensing probes and 2 mm between the voltage-sensing and current-carrying probes, respectively. The HCl-induced injury led to a decrease in the lung bioimpedance between the frequency range of 0.1 to 300 kHz. Moreover, the magnitude of bioimpedance decreased with increasing volume of acid instilled into the lungs. The reduction in bioimpedance of HCl-injured lung tissue may be due to the replacement of air with fluid and cell debris in the airspace and an increase in the alveolar septa thickness, which collectively provide larger conductive pathways for electrons and ions. Notably, the R₀−R_{∞} level decreased substantially in acid-injured lungs compared to native intact lungs. While the bioimpedance reduction was significant between lungs treated with 1 mL and 3 mL of HCl, the smaller difference observed between lungs treated with 3 mL and 5 mL suggests fluid saturation, which limits further changes in conductive pathways. Similar to the bioimpedance, the phase angle of the tissue decreased with increase in the level of HCl volume up to 3 mL due to disruption of the cell membrane integrity and overhydration. However, there was no significant difference between the phase angle of the injured lungs treated with 3 mL and 5 mL HCl volumes. In Cole-Cole fittings, the deviation from depressed semi¬circle behavior was increased with an increase in delivered HCl volume (FIGS. 38A-38B).

Furthermore, the bioimpedance of local tissue regions of acid-injured lungs treated with 2 mL HCl solution (concentration: 0.1 N, pH level: 1) was measured. The spacing between the outer electrode probes was kept constant at 20 mm, while the inner electrode probes were positioned in a linear fashion with outer electrodes on the random regions. The distance between the inner electrode probes was 1 mm, and the bioimpedance was measured at a fixed frequency of 2 kHz. The injured regions were identified via the change in the color of lung tissue, with the nightingale-brown brown regions being primarily affected by the HCl solution. The results demonstrated different bioimpedance values across the lung regions. Moreover, although certain regions showed less surface discoloration than others, they exhibited a lower bioimpedance level, which could be due to more subpleural injury and internal damage that is not readily visible at the surface. Collectively, the results highlight the functionality of the bioimpedance measurements in detection of locally injured lung tissues impacted by acid.

The trachea is composed of a C-shaped cartilaginous ring (i.e., anterior side) and muscular tissue through which the trachea is connected to the esophagus (i.e., posterior side). It was hypothesized that this unique tissue composition could create a spatially varying bioimpedance across the trachea tissue cross-section. To test this hypothesis, the bioimpedance was measured along the anterior (i.e., cartilage-rich) and posterior (i.e., muscle-rich) sides of the rat trachea. The current-carrying and voltage-sensing probes were positioned in linear fashion on both sides, and the bioimpedance values were measured. Similar to lung tissue bioimpedance measurements, a sinusoidal current with a magnitude of 100 μA and a frequency range of 0.1-300 kHz was injected into the trachea via current-carrying probes, and the response was measured using voltage-sensing electrode probes. The spacing between the inner probes and the spacing between the inner and outer probes were set to 1 mm and 2 mm, respectively.

The bioimpedance of both anterior and posterior sides showed a frequency-dependent trend in the frequency sweep measurements. Notably, the bioimpedance magnitude of the cartilage-rich tissue was higher than that of the muscle-rich tissue across most frequencies (range: 0.1 to 250 kHz). The higher bioimpedance of cartilage-rich tissue could be due to the lower water content in cartilage tissue than in muscle tissue. At very high frequencies (range: 250 to 300 kHz), the bioimpedance values of both sides converged to the same range. This convergence may be due to an increase in permeability of the cell membrane to electrical current and the contribution of intra-cellular fluid in overall bioimpedance at these frequencies [67]. The electrical response in cartilage-rich and muscle-rich tissues was resistive (|R|<|X|) below frequencies of 100 kHz and 260 kHz, respectively (FIGS. 39A-39C). Below the frequency of 6 kHz, the phase angle values of the muscle-rich side were higher than those of the cartilage-rich side; however, this trend reversed at frequencies above 6 kHz (FIGS. 39A-39C). This could be attributed to differences in cell density or water content between these tissues. In Cole-Cole plots, both anterior and posterior sides exhibited a depressed semi-circle behavior, and the magnitude of R₀−R_{∞} for the cartilage-rich part was larger than that of the muscle-rich part.

Since air is nonconductive to electrical current, the current flows primarily through the trachea wall tissue. Consequently, the depressed semi-circle region observed in the plots obtained from the airway tissues makes the Cole Cole plot suitable for the fitting. The transmission of the electrical current flow through the trachea tissue was further validated through computer simulation using COMSOL software (FIGS. 40A-40F). Specifically, the simulation results indicated that the electrical current travels from the electrode probes through the tissue region to reach the bottom part of the trachea. Having the experimental data fitted on the Cole Cole Equation, the Cole Cole parameters of the anterior and posterior sides of the trachea were determined. Compared to lung tissue, the bioimpedance of the trachea on both parts showed agreement with the Cole-Cole model. The bioimpedance behavior of both cartilage-rich and muscle-rich tissues was consistent with the Cole-Cole model (R2>98). The a, τ₀, and R₀−R_{∞} parameters for the cartilage-rich tissue were higher than those of the muscle-rich tissue. Furthermore, the a value for cartilage-rich tissue was closer to 1, suggesting greater tissue homogeneity across the cartilage-rich tissue compared to that of the muscle-rich tissue.

FIG. 40A is a 3D model of the trachea, showing the positions of the electrode probes on its outer surface. FIG. 40B is side and front views of the trachea. FIG. 40C shows simulation parameters. FIG. 40D shows the current density normal (J) distribution within the tracheal tissue. showing the current primarily flows through the tissue region, with no current present in the luminal air space. FIGS. 40E and 40F are cross-sectional views of the electrical current flow in two different outlines (cutline 1: at the terminal electrode; cutline 2: at the centerline of voltage-sensing electrodes). The simulations confirm that the electrical current is concentrated in the tissue region. a: Electrical conductivity; er: relative permittivity, J: current density normal.

Tissue bioimpedance comprises a resistive component (real part), primarily attributed to the extracellular and intracellular fluids, and a reactive component (imaginary part), mainly due to the capacitance created by the lipid bilayer cell membranes. Decellularized lung and trachea tissues were prepared to investigate how a change in cellular composition can contribute to the change in the overall bio-impedance of these tissues. To this end, rat lung and trachea tissues were treated with a series of decellularization solutions (SDS and Triton X-100), and their bioimpedance values were measured. During the measurements, the spacing between the voltage-sensing probes was 3 mm, while the distance between the voltage-sensing and current-carrying probes was set to 2 mm. For trachea tissue measurements, these distances were set to 1 mm and 2 mm, respectively.

Decellularization of lung sections led to the generation of translucent tissues. The histological images of decellularized lung sections showed no discernable cellular content and nuclear materials. The bioimpedance of decellularized lung sections was substantially lower than that of native intact sections. The decreased bio-impedance could be due to the removal of cells and an increase in the permeability of decellularized tissues. Notably, the bioimpedance of the decellularized lung tissues showed a lower dependency to the frequency as a result of the removal of cell membranes (FIGS. 41A-41C). Moreover, the removal of cellular components led to the disappearance of a depressed semi-circle in the Cole-Cole plots in decellularized tissue sections (FIGS. 41A-41C). A reduction in both resistance and reactance was observed in the decellularized tissues (FIGS. 41A-41C). This indicates that the cell membrane contributes significantly to resistance, reactance, and the overall bioimpedance. Similar to lung tissue, decellularized tracheas showed translucency, and the histological analysis confirmed the removal of most of the cellular components (FIGS. 42A-42B). The measured bioimpedance of decellularized tracheas was lower than that of native intact tracheas for both cartilage-rich and muscle-rich sides. The bioimpedance behavior of decellularized tracheas at both sides deviated from depressed semi-circle behavior (FIGS. 43A-43F). Moreover, the strength of resistance (R₀−R_{∞}) decreased on both sides of the trachea following the decellularization process.

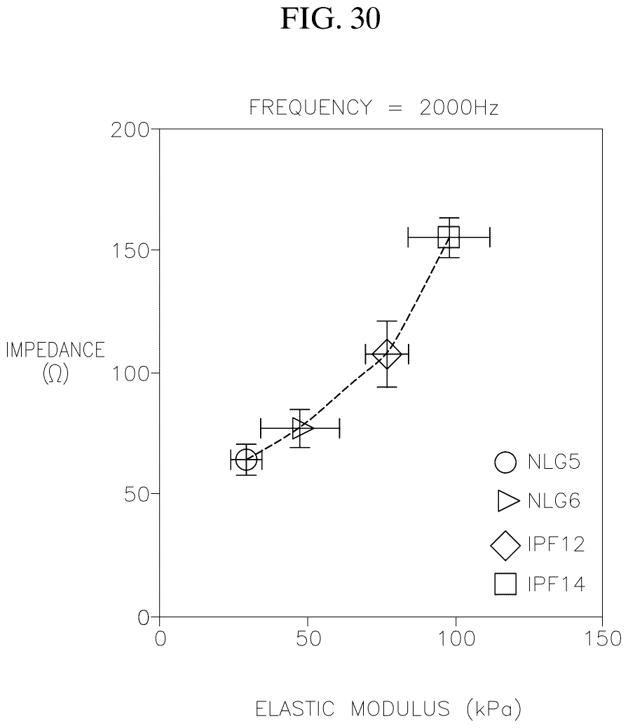

Further investigation characterized the bioimpedance of decellularized healthy and fibrotic human lungs to investigate the role of ECM (i.e., tissue fibrosis) on the lung tissue bioimpedance (FIG. 30). The tissues were obtained from two healthy donors (labels: NLG5 and NLG6) and two individuals with end-stage lung disease diagnosed with IPF (labels: IPF12 and IPF14). The tissues were cut into sections (thickness: ~2 mm thickness) and decellularized using a series of CHAPS-based decellularization solutions. During the bioimpedance measurements, the spacing between the voltage-sensing probes was set at 2 mm, while the distance between the voltage-sensing and current-carrying probes was maintained at 4 mm. The results showed higher bioimpedance levels in fibrotic tissues compared to healthy lungs throughout the frequency spectrum of 0.1-300 kHz. Moreover, the bioimpedance of both healthy and fibrotic lung tissues showed frequency-independent bioimpedance due to the removal of the cellular component. Specifically, there was no significant difference between the reactance and phase angle values of healthy and fibrotic lungs (FIGS. 44A-44D). Since the cells were absent in both healthy and fibrotic tissues, the higher levels of bio-impedance in fibrotic tissues could be due to ECM composition, resulting in high resistance to alternating current flow.

FIG. 44A is a series of photographs of decellularized healthy and fibrotic human lung tissues. Healthy tissues (NLG5 and NLG6) were obtained from two healthy donors and fibrotic tissues (IPF12 and IPF 14) collected from two patients with end-stage lungs diagnosed as idiopathic pulmonary fibrosis (IPF). Resistance (44B), reactance (44C), and phase angle (44D) plots of healthy and fibrotic lung tissues across the frequency spectrum (range: 0.1 to 300 kHz) are shown.

Mechanical stiffness is defined as tissue resistance to deformation in response to an applied force, and it is often represented by the elastic modulus (E). Similar to bioimpedance, tissue stiffness is inter-connected with the ultrastructure and composition of ECM in the tissue. Therefore, the correlation between tissue stiffness and bioimpedance in native and fibrotic lung tissues was investigated. To this end, tensile testing was used to assess the mechanical stiffness (i.e., elastic modulus) of both healthy and fibrotic tissues. The measured elastic moduli from stress-strain curves showed higher stiffness in fibrotic tissues than healthy tissues. Plotting bioimpedance values of healthy and fibrotic tissues at 2 kHz against the corresponding elastic moduli indicated a positive correlation, with stiffer tissues showing higher bioimpedance values. (FIG. 30). The increase in both bioimpedance and stiffness of fibrotic tissues could be due to a change in composition and structural organization in these tissues.

Biological tissues are comprised of two electrically conductive components, including intracellular and extracellular fluids, that are separated by a cell plasma membrane. The membrane acts as a constant element phase (a frequency-dependent capacitor), being an insulator at low frequencies but permeable to electrical currents at high frequencies. As a result, biological tissues behave as conductors, dielectrics, or insulators depending on their composition and structural organization. Therefore, the electrical impedance behavior of biological tissue across a certain frequency spectrum can provide information about their properties and physiological states and offer diagnostic and prognostic insights. In lung diseases, the intracellular and extracellular compositions of the lung alter with the progression of the disease. In procedures consistent with embodiments of the present invention, the bioimpedance of ex vivo lung tissues was investigated by placing electrode probes on the surface of these tissues (FIGS.). The findings suggest that electrical bioimpedance can effectively characterize the lung tissues and differentiate various tissue states.

First, the bioimpedance of intact rat lung tissue at different electrode probe spacings (FIGS. 29A-29H) and lung internal pressure levels was investigated. The probe configuration experiment showed that the lung bioimpedance level was decreased by enlarging the spacing between the current-carrying and the voltage-sensing electrodes (b) in the frequency sweep test. This counter-intuitive observation is consistent with theoretical models and electrical resistance measurements of semi-infinite 3D materials. Specifically, the electrical field generated between the current-carrying probes spreads into a 3D sample in all directions perpendicular to the probes, which causes the resistance to be inversely proportional to the probe spacing (FIGS. 45A-45B). Overall, the lung tissue showed a resistive response (|R|>|X|) to the injected current at frequencies below 100 kHz, which caused the probe spacing to have a significant impact on measured bioimpedance. In the bioimpedance assessment of gelatin-based hydrogel samples with varying thickness levels, the results indicated that as the sample thickness decreased, the influence of probe spacing on both resistance and bioimpedance magnitudes diminished (FIGS. 34A-34I). Moreover, the bioimpedance showed dependency on the volume of air in the alveolar airspace. The bioimpedance changes with P_{ALV} were consistent with literature data and showed an increase with an increase in pressure and volume. In both experiments, the bioimpedance of intact lungs showed a depressed semi-circle behavior of the lung and high agreement with the Cole-Cole function.

Next, the bioimpedance of lung tissue impacted by an acid solution was measured to investigate whether the bioimpedance measurement can differentiate native tissue and acid-injured lungs. The goal of acid-induced lung injury was not to recapitulate the stereotypical presentation of the ARDS condition, but rather to create an acute injury in the lung tissue and assess its bioimpedance change. The acid component of gastric aspirates is frequently modeled by intratracheal instillation of acids in animal models. Specifically, The HCl solution (concentration: 0.1N, pH level: 1) has been used to mimic gastric aspiration-induced lung injury. As the presence of local fluid can impact regional bioimpedance, the accumulation of fluid in the lung, as observed in gastric acid-induced lungs, can be detected through a reduction in bioimpedance. In the present instance, acid-induced injury was created through intratracheal instillation of HCl acid into the lung tissues. The measurements showed that injury led to lowered bioimpedance in injured tissue due to the presence of fluid in alveolar airspace and swelling of the alveolar septa. Moreover, local measurements of bioimpedance in lungs with spatially distributed HCl highlighted the capability of the bioimpedance assessment in differentiating non-injured and injured regions. Since acute lung injury is characterized by severe pulmonary edema in the lungs, monitoring of lung fluid content via bioimpedance measurements can be used in early diagnosis of acute lung injury and timely treatments of the patients.

Previously, the change of bioimpedance of airway tissue was measured after creating a localized injury on the epithelial layer of ex vivo rat trachea tissue. It was found that disruption of the airway epithelium leads to reduced bioimpedance measured at the airway lumen due to local disruption of epithelial cells, which are the main contributors to the hinderance of alternating current flow at low frequencies. It was hypothesized that the spatial composition across the trachea ring leads to spatially distributed bio-impedance. The bioimpedance characterization of the trachea at the anterior (i.e., cartilage-rich) and posterior (i.e., muscle-reach) sides confirmed this hypothesis, as both sides showed different bioimpedance levels. Specifically, the results indicated that the cartilage-rich side showed higher bioimpedance levels compared to muscle-rich side. This observation demonstrates the utility of the bioelectrical impedance measurements in the regional evaluation of heterogeneous tissues.

Since both cells and ECM contribute to overall tissue bioimpedance, the impact of the removal of cells on the bio-impedance of lung parenchymal and trachea tissues tissues was studied. To this end, the tissues were treated with a series of decellularization solutions, and their bioimpedance values were measured. The results showed a significant reduction at all frequencies in both decellularized trachea and lung parenchyma tissues compared to native tissues. Bioimpedance measurements are useful not only for characterizing injured or diseased lung tissue but also for providing quantifiable data that supports innovative tissue engineering approaches aimed at treatment of severely injured lung tissues. For instance, the bioimpedance measurement technique can be utilized in “whole lung bioengineering”, which involves generating acellular lung scaffolds (i.e., decellularization) and seeding new primary and stem cells onto the acellular scaffold (i.e., recellularization). The bioimpedance of ex vivo lungs can be monitored during the decellularization of tissues to ensure the complete removal of cellular components. Additionally, the method can be used to monitor acellular tissue scaffolds during the attachment, growth, proliferation, and differentiation of newly seeded cells, and to evaluate tissue vascularization during tissue culture in the bioreactor.

Further, to investigate the role of ECM on the bioimpedance, the bioimpedance of decellularized healthy and fibrotic human lung tissues were studied (FIG. 30). The measurements showed higher bio-impedance in fibrotic decellularized tissues compared to the healthy tissues. This observation was connected to the compromised ECM of the fibrotic tissues due to excessive fibrillar collagen deposition during ECM remodeling. It is believed that fibrotic tissues show greater electrical resistance to current flows compared to healthy tissues. However, a few studies have reported contrasting results for bioimpedance measurements of fibrotic tissues in vivo. During in vivo measurements, severe inflammation, local edema, and lowered cellular content may contribute to reduced bioimpedance of fibrotic tissues compared to healthy tissues. Given that increased mechanical stiffness is a hallmark of fibrotic tissues, the mechanical and electrical evaluations of healthy and fibrotic lung samples showed a positive correlation between tissue stiffness and bioimpedance. This observation can suggest the potential utility of bioimpedance measurements as a diagnostic tool for pulmonary fibrosis. Moreover, the present invention can help investigate how the ECM alteration in fibrotic lung tissues influences the bioimpedance. Future work can explore bio-impedance measurement across a variety of organs and tissues with different levels of fibrosis in in vivo conditions.

It is important to recognize the influence of geometric factors, such as electrode probes' arrangement, probe size, probe spacing, and the shape and dimensions of the tissue on the bioimpedance measurements. For instance, in the four-electrode impedance measurement method, the arrangement of electrodes in square and linear configurations can produce different results due to variations in the electric field distribution between the probes.

To ensure a reliable comparison between the experimental groups, the probes were arranged in-line, and the spacings between the probes were precisely controlled using XYZ micromanipulators and a camera. As the curved shape of the lung lobes can introduce variability in tissue thickness across different regions, the measurements were taken on the same anatomical location of the lungs (i.e., right middle lobe) and under consistent lung pressure and volume to ensure a reliable comparison between the experimental groups. In future studies, the electrodes can be securely fixed or printed onto fixture to minimize the variations caused by tissue thickness and probe spacing arrangement. Additionally, depending on the organ type and probe geometry, a geometric correction factor can be calculated and applied to the measurements to ensure accurate bioimpedance assessment.

Tissue deformation caused by electrode probes and the pressure generated by the electrode probes around the probe-tissue interface are critical factors in the bioimpedance measurements. These factors become particularly important when probe diameter size becomes smaller than 5 mm. It is believed that a high indentation (i.e., high contact force) can lead to squeezing the fluid out of the tissue around and between the probes which results in a decrease in measured bio-impedance. It is suggested that using larger electrodes that are spaced apart can reduce surface conductive liquid effects and contact impedance. Notably, the four-probe measurements are generally preferred as they eliminate impact caused by probe-tissue contact impedance on the measurements. For the present experimental setup, the XYZ micromanipulators were used to precisely translate and position the electrodes onto the tissue surface and compress the tissue by 500 μm. Future studies can be designed to measure bioimpedance while simultaneously recording the applied force on the target tissue surface using a force sensor to ensure consistent measurements.

It should be emphasized that the Cole-Cole function was applicable only over a certain frequency range in the bioimpedance measurements of lung and the trachea tissues. In both tissues, a linear trend appeared in the low-frequency region of the Cole-Cole plots. This region is often attributed to the properties of the electrode probes, the tissue surface, and the probe-tissue interface. The Cole-Cole function assumes a characteristic semi-circular arc in the Cole-Cole plot; however, it does not adequately explain deviations such as the linear trend observed at low frequencies. In these analyses, to apply the Cole-Cole function to the measured data, low-frequency data exhibiting linear trends were excluded, and the fitting analyses were focused on the semi-circular region where the Cole-Cole function shows high accuracy. Future studies could explore incorporating more complex models, such as a series connection of two Cole-Cole circuits, to accurately capture the entire frequency spectrum.

Variations in bioimpedance measurements across different studies can be attributed to factors such as the measurement device used, the number of electrode probes, probe geometry, measurement setup, the physiological condition of the tissue, and the moisture content of the tissue surface (FIG. 46). Consistency in these parameters is critical for achieving reliable and reproducible bioimpedance data across different experimental conditions. For instance, as demonstrated in the present investigation, the spacing and arrangement of the probes can directly influence the distribution of the electric field, which impacts the magnitude of the bioimpedance values (FIG. 29A-29H). For lung tissues, respiratory pressure and volume affect the measured bio-impedance; therefore, these parameters can be monitored when measuring the electrical properties. Moreover, as the moisture content of the tissue surface greatly influences its bioimpedance, it is important to maintain consistent moisture and hydration levels across all experimental groups to ensure reliable comparisons.

In clinical settings, bioelectric signals originated from the human body, such as electromyograph (EMG) and electrocardiograph (ECG) signals, can contribute to increased noise levels during lung bio-impedance measurements. Specifically, diaphragmatic and cardiac activities may interfere with the accurate detection of impedance changes in the lungs. To mitigate these effects and achieve a robust bioimpedance output from the measurement device, signal processing techniques, such as adaptive filtering or noise cancellation algorithms can be employed. For instance, the cardiovascular bioimpedance change can be removed from the output signals using the low-pass filtering or frequency separation methods such as empirical mode decomposition (EMD). An effective approach to improving the analysis of impedance changes within the lung is to select a smaller region of interest for the measurements. Collectively, these approaches can allow clinicians to achieve more accurate lung bio-impedance assessments, thereby enhancing diagnosis and monitoring of respiratory conditions.

The bioimpedance measurement through direct electrode probe-tissue contact can be translated to clinics to aid in the diagnosis, evaluation, and treatment of lung injury and diseases. Specifically, the electrode probes can be utilized in various tools and devices to locally measure the bioimpedance of lung tissue in operating rooms. To this end, the electrode probes can be integrated into a robotic surgical tool and provide real-time bioimpedance of the tissue during open chest surgery, video-assisted thoracoscopic surgery (VATS), and minimally invasive robot-assisted surgery (RMIS) and locally evaluate lung tissue quality. For example, the integration of bioimpedance probes with surgical tools can allow intraoperative detection of tumor boundaries in real-time with high spatial resolution to facilitate effective tumor resection while maximizing the preservation of healthy tissue. Moreover, bioimpedance assessment can aid in diagnosing lung pathologies that are typically challenging to distinguish without performing a tissue biopsy. In the future, the electrode probes can be integrated into a bronchoscope and intratracheally deployed into the patient's body to locally evaluate tissue bioimpedance of pathologic lung tissue in situ. For instance, flexible thin film-based electrodes can be attached to the bronchoscope tip and be introduced into the respiratory tract of a patient with airway injury to allow minimally invasive quantification of airway tissue injury. Moreover, the integration of electrodes can allow surgeons to establish the accurate orientation of regular or robotic bronchoscopes when advancing to the smaller airways.

Future studies might aim to enhance the device configuration and measurement conditions to enable clinical translations. Although the bioimpedance of fresh tissues was measured within 30 min of tissue being exposed to air, the condition can be different from in vivo situations. For example, during in vivo measurement, the dynamic ECM and cell activities, blood circulation, local edema, and ventilation can impact the bioimpedance measurement outcomes. Moreover, it was observed that the tissue surface desiccation over time can impact the electrical measurement. To minimize tissue desiccation, the measurements were taken immediately after explantation. In the future, the ex vivo measurements can be conducted in environments with maintained ambient humidity at 100% to prevent dehydration during testing. Further studies can also investigate a broader frequency spectrum, extending up to the GHz level, while ensuring a safe and clinically relevant current magnitude (current level<100 μA) is injected into the tissue. Such analysis may provide a clearer picture of the tissue response to electrical current and enhance the diagnostic capabilities of the impedance characterization.

In conclusion, the electrical properties of tissues, measurable as bioelectrical impedance (bioimpedance), are closely linked to their health and pathological conditions. In this study, the four-probe measurement technique was employed to characterize the electrical properties of ex vivo lung tissues by recording their bioimpedance under different conditions. The results demonstrated that bioimpedance can serve as a biomarker for the accurate and rapid determination of tissue injuries and diseases, such as fibrosis. This electrically-based technique can be extended to evaluate lung conditions during minimally invasive surgery or to monitor ex vivo lungs in a bioreactor during regeneration. Beyond the lungs, this approach can be used to diagnose pathologies in various organs and tissues, aiding in the treatment of diseases and injuries.

### Methods:

Tracheas and lungs were isolated from Sprague-Dawley rats (SAS SD Rats; Charles River Laboratories; body weight: 200-250 g, total animals: 20). All animal experiments were conducted with approval from the Institutional Animal Care and Use Committee (IACUC) at Stevens Institute of Technology (Protocol number: 2023-002 (AP)). All animal care was performed under the Guide for the Care and Use of Laboratory Animals standards published by the National Institutes of Health (NIH) and according to the Animal Welfare guidelines and regulations.

The rats were anesthetized via inhalation of 2.5% isoflurane (Cat. Number: VED1350, Penn Veterinary Supply) in the air for 20 min in an induction chamber using a vaporizer (SomnoSuite®, Kent Scientific). The depth of anesthesia was confirmed by evaluating the pedal reflex. The animal was then transferred to the surgical board and connected to an anesthesia nose mask. To prevent vascular clotting in the lung tissue, 0.5 mL of 1,000 units/mL heparin was injected into the tail vein. To isolate the lungs, surgical scissors were first used to enter the peritoneal cavity by a midline laparotomy. Next, the animal was euthanized by transecting the inferior vena cava (IVC) and further exsanguination. Before rupturing the thoracic cavity, an incision was made cranially past the manubrium into the neck to expose the surface of the trachea. The trachea was then cannulated with a tracheal cannula (diameter: 2 mm; 73-2727, Harvard Apparatus) and secured with a suture (size: 4.0, Matrix Wizard). 1 mL of air was then injected into the lungs via a syringe connected to the trachea cannula to partially inflate the lungs. Midline thoracotomy was then performed to cut between the ribs and open the chest. The lungs were removed by cutting connective tissues around the lungs and trachea. The lungs were rinsed with 1×phosphate-buffered saline (1×PBS, pH=7.2, Gibco™) prior to bioimpedance measurements and further tests.

To isolate only the trachea from the rat, after euthanasia, a midline incision was made in the anterior neck to expose the surface of the trachea. Midline thoracotomy was then performed by making an incision in the chest wall to reach the trachea end connected to the lungs' bronchi. To extract the trachea, both ends of the trachea were cut using scissors. The trachea was rinsed with 1×PBS before the bioimpedance measurements and further analyses.

The human lungs were procured from the New York Organ Donor Network (NYODN) under a protocol approved by the Institutional Review Board at Columbia University. The lungs were from two healthy donors with no history, diagnosis, or evidence of smoking and lung diseases and two patients with end-stage lungs diagnosed as idiopathic pulmonary fibrosis (IPF) lungs by transplant surgeons. Upon harvest, the lungs were cleared of blood and immediately stored at 80° C. until use. The lung tissues were partially thawed and sectioned into 2 mm-thick slices using a vibratome device (VT1200S, Leica). The sections were washed in 2×PBS for 15 min and decellularized four times with 8 mM 3-[(3-Cholamidopropyl)dimethylammonio]-1-propanesulfonate (CHAPS, duration: 2 h), each followed by distilled H₂O (duration: 5 min) and 2×PBS (duration: 15 min). Before the bioimpedance measurements, the decellularized sections were kept in Dulbecco's phosphate-buffered saline (10×, Cat. Number: D1283, Sigma Life Science) solution. The sections were cut in rectangle shapes (2 cm height×1 cm width×2 mm thickness), and the bioimpedance of the tissue sections was measured at room temperature.

The acid-induced lung injury was created according to protocol used in previous studies. To induce injury in the lungs caused by acid, different volumes (1, 3, and 5 mL) of hydrochloric acid (HCl) solution (0.1 N, pH level: 1) were intratracheally instilled into the lung via a 10 mL syringe connected to the trachea cannula. After each HCL instillation, 0.5 mL of air was injected through tubing to ensure that HCl had reached the distal parts of the lungs. The bioimpedance of injured lungs was measured 10 min following HCl administration. Native intact lungs were used as a control group for comparison.

Rat lungs were connected to a small animal ventilator (PhysioSuite, Kent Scientific) through the endotracheal cannula and ventilated with a tidal volume of 7 mL/kg at a rate of 70 breaths per minute (bpm). During ventilation, pressure and flow were monitored through integrated sensors in the ventilator and tubing. The volume for each breath was determined by the area under the flow-time curve.

To decellularize the rat lungs, lungs were cut into slices (thickness: −3 mm) using a sharp blade and were washed with 1×PBS. The sections were transferred to deionized (DI) water and were decellularized using a series of decellularization solutions. The lung sections were first placed in 0.2% sodium dodecyl sulfate (SDS, Cat. Number: 0227, VWR) solution for 48 h at 4° C. on a tube revolver. Next, the sections were washed with 1×PBS and placed in 0.1% Triton X-100 solution (Cat. Number: M143, VWR) for 96 h at 4° C. on a tube revolver. The decellularization solutions were changed every 24 h. The sections were then rinsed with DI water and 1M sodium chloride (NaCl) solutions for 15 min. All samples were washed with DI water prior to bioimpedance measurements.

To decellularize the trachea, the residual connective tissues were removed after extracting the trachea from the rat. The tracheas were decellularized in a series of decellularization solutions: First, the tracheas were placed in a 3% Triton X-100 solution at 4° C. for 48 h on a tube revolver. The decellularization solutions were changed every 24 h. Subsequently, the tracheas were exposed to a 0.1% Triton X-100 solution at 4° C. for 12 h on a tube revolver. Next, the tracheas were treated with 96% DI water and 4% ethanol for 2 h. Finally, the tracheas were immersed in a 0.9% NaCl solution in DI water for 15 min. The trachea samples were washed with DI water prior to bioimpedance measurements.

Gelatin-based tissue phantoms were prepared by dissolving gelatin powder (G2500, Sigma Aldrich) in 1×PBS at 70° C. (concentration: 5 w/v) and physically crosslinking the solutions at room temperature for 10 min. To prepare phantoms with 50 μm and 1 mm thicknesses, the rectangular molds (width: 12 mm) were created by depositing the layers of polyimide tape (thickness: 25 μm; Kapton® Tape) on a glass slide and pouring the gelatin solution in the molds for crosslinking. To create gelatin phantoms with 5 mm thickness, polydimethylsiloxane (PDMS) molds (ratio of prepolymer to crosslinker: 10:1; width: 1 cm) were created, and the gelatin solution was poured into the molds for crosslinking.

The four probes method with alternating current (AC) was employed to measure the bioimpedance of the lung and trachea tissues. Bio-impedance values were collected using a Keysight impedance analyzer (model: E4980AL-032; frequency range: 20 Hz-300 kHz; Keysight Technologies). For all measurements, the probes were aligned along a straight line on the surface of the tissue samples, and the impedance analyzer was set to apply a sinusoidal current of 100 μA as the electrical excitation. The bioimpedance values were measured using electrode interfaces made of tungsten (SE-TL, Lucas Signatone; length: 30 cm; shank diameter: 0.6 mm; tip diameter: 5 μm). Frequency (w) sweep measurements were conducted in the frequency range from 0.1 to 300 kHz, and resistance (R, real part of bioimpedance) and reactance (X, imaginary part of bioimpedance) were collected for 50 discrete frequencies within this range. For single frequency-point measurements, a frequency was set to a specific value, and resistance and reactance were measured. The magnitude of bioimpedance (|Z|) was calculated from resistance and reactance values:

Z=R+jX (1)

where

|Z|=R²+X²Ω (2)

The phase angles (φ) were calculated using equation (3) below:

$$ϕ = {tan}^{- 1} \frac{X}{R} * \frac{π}{180} ( degree ) \qquad ( 3 )$$

Prior to all the measurements, the instruction protocol was followed for the Keysight LCR Meter (E4980AL) to remove the residual impedance and other parasitic effects within the device and from the measurement path. To this end, open and short circuits were created and the open and short corrections were made in the device setup according to the manufacturer documents. Briefly, in the setting panel, in the “measurement setup” windows, the “Correction” option was selected. The “Open” and “Short” options were turned on, and the “Measure Open” and “Measure Short” procedures were performed separately for both options. In the “Short Correction” step, a short circuit was created within the all the probes. The correction was performed on all frequency points. Following the correction procedure, the electrical impedance of a known resistance-capacitance (RC) circuit containing resistor (10 k52) and capacitor (100 nF) in a parallel mode was measured along with the resistance and capacitance values across the frequency range (0.1-300 kHz). The impedance (Z) values obtained by the device were closely aligned with the theoretical Z values of the RC circuit for all the frequency points (FIGS. 31A-31C).

The measurements were performed through controlled positioning the electrode probes on the surface of the tissues. To ensure precise placement of the probes, XYZ micromanipulators were used, and the contact was visually monitored with the naked eye throughout the process. Once initial contact was established, approximately an additional 500 μm of indentation depth was applied to ensure secure and stable probe-tissue contact while minimizing the risk of tissue surface injury. To maintain consistency and achieve a reliable comparison between the experiment groups, the same experimental setup was used (probe arrangement, probes spacing, and current magnitude), and measurements were performed on the same the anatomical location of the lungs (i.e., right middle lobe) for the experimental groups. The spacing between the probes were selected based on previous studies as well as the size of the rat lung and trachea. Moreover, to minimize tissue desiccation, the bioimpedance of fresh lung and trachea was measured immediately after isolation and washing with DI water. For treated tissues, such as decellularized lung and trachea, measurements were taken immediately after DI water washing and blotting them dry.

The Cole-Cole model mathematically describes the depressed-semicircle characteristics of biological tissue [67]. The experimental bioimpedance data for lung and trachea tissues were fitted with the Cole-Cole equation as described below:

$$Z = {R}_{∞} + \frac{{R}_{0} - {R}_{∞}}{1 + {( j ω {τ}_{0} )}^{a}} \qquad ( 4 )$$

where R₀ is the resistance at zero frequency and R_{∞} is the resistance at the infinite frequency. a is a dimensionless numerical distribution parameter with a value between 0 and 1. When a is close to 1, the sample composition is more homogenous. τ is the characteristic time constant corresponding to a characteristic frequency (f_{c}), which is the frequency at which the magnitude of reactance is maximum (f_{c}=ω_{c}/2π=1/πτ). Given that the Cole-Cole equation represents a non-linear relationship between bioimpedance and frequency, and it generates complex numbers, by applying j^{α}=cos(απ/2)+j sin(απ/2) expression with j=(−1)^{1/2} on Eq. (4), the real and imaginary parts were decoupled, and the Cole-Cole equations were reformulated:

$$Z = R + Xj = {R}_{∞} + \frac{{R}_{0} - {R}_{∞}}{1 + {( ω τ )}^{a} {j}^{a}} = {R}_{∞} + \frac{{R}_{0} - {R}_{∞}}{1 + {{( ω τ )}^{a} [ cos ( \frac{a π}{2} ) + j sin ( \frac{a π}{2} ) ]}^{a}} \qquad ( 5 )$$

$$where$$

$$R = {R}_{∞} + \frac{( {R}_{0} - {R}_{∞} ) ( 1 + {( ω τ )}^{a} sin ( \frac{a π}{2} ) )}{1 + 2 {( ω τ )}^{a} cos ( \frac{a π}{2} ) + {( ω τ )}^{2 a}} \qquad ( 6 )$$

$$X = \frac{( {R}_{0} - {R}_{∞} ) {( ω τ )}^{a} sin ( \frac{a π}{2} )}{1 + 2 {( ω τ )}^{a} cos ( \frac{a π}{2} ) + {( ω τ )}^{2 a}} \qquad ( 7 )$$

R₀, R_{∞}, a, and τ values were calculated by fitting the R and X equations to experimental data. Since the Cole-Cole function is primarily designed to describe the depressed semicircular region of the Cole-Cole (R-X) plot, the measured data outside this region were excluded for the purpose of fitting. The fitting was achieved concurrently for both the measured resistance and reactance values. All fittings were performed on the average measured values for each experiment group. The best fit was determined by minimizing the Least Squares (LSQ) function, defined as:

$$LSQ = {\sum }_{i = 1}^{N} [ {( {R}_{meas} - {R}_{fit} )}^{2} + {( {X}_{meas} - {X}_{fit} )}^{2} ] \qquad ( 8 )$$

Where N is the number of frequency data point and Rₘₑₐₛ. and Xₘₑₐₛ are the measured resistance and reactance values, respectively, and the R_{fit} and X_{fit} are the fitted resistance and reactance values obtained by the Cole-Cole function. To achieve the best fit, the optimization process was performed using the sequential least squares programming (SLSQP) algorithm in Python. The R² values were calculated to evaluate the quality of the best fit.

To obtain histological images of fresh lungs at varying alveolar pressures (P_{ALV}), first, the target pressures (1, 5, and 15 cmH₂O) were achieved using a syringe connected to a pressure sensor. When the target pressure was reached, the trachea was sutured to maintain the pressure, and the trachea cannula was removed. The lungs were then submerged in 4% neutral buffered paraformaldehyde solution in 1×PBS (pH: 7.4) at 4° C. overnight and transferred to a 70% ethanol solution. The fixed tissues were dehydrated in a series of isopropyl alcohol (IPA) solutions (concentrations: 80%, 85%, 90%, 95%, and 100%) and Citrisolv™ solution (Cat. Number: 1061, Decon) before paraffin embedding. The tissue samples were then embedded in paraffin and were cut into thin slices (thickness: 5% mm). The tissue slices were deparaffinized with Citrisolv™ solution and rehydrated with a series of IPA solutions (concentrations: 100%, 95%, 90%, 85%, 80%, 70%, and 50%) and DI water, respectively. The lung and trachea sections were stained with hematoxylin and eosin (H&E) staining kit (Cat. Number: ab245880, Abcam) and mounted with mounting medium (Permount™, Cat. No. SP15-100, Fisher Scientific) for better preservation. The same fixation, sectioning, and staining procedures were used to obtain the H&E images of acid-injured and decellularized lungs, as well as native and decellularized trachea tissues.

To quantitively assess the impact of HCl on the lungs, the area of alveolar space and alveolar septa thickness were used to compare native intact (control) and injured lungs. The histology images of native and injured lungs were imported to the ImageJ software (NIH). The measurement scale was set using a known distance, i.e., the image scale bar, as a ruler. The scale of the image was set using the “Set Scale” function. Random regions were selected from images, and the septa thicknesses were measured by drawing a line across the alveoli septa and using the “Analyze” function. To measure the alveolar airspace area, the H&E images were converted into binary images, and the alveolar tissue regions were selected by adjusting the threshold values using the “Threshold” function. The area of alveolar airspace was determined using the “Analyze Particles” function.

The healthy and fibrotic lung sections were cut in rectangular shapes (2% cm height×1% cm width×2% mm thickness) and were stored in 10×PBS solution prior to the mechanical tests. Tensile properties of the tissue slices were measured using a universal testing machine (Instron 5965; cell load: 5% kN; Instron Inc., USA) at a crosshead speed of 5% mm/min. Sandpaper was used at both ends of the tissue slices to hold the samples firmly on the grips. The elastic modulus of tissue slices was determined by calculating the slope of the linear region (σ=Eε, where σ is stress, E is elastic modulus, and C is strain) of the stress-strain curve at a low strain region (0.5%%).

COMSOL Multiphysics software (Version 6.0) was used to simulate the electrical current propagation within the trachea tissue (FIGS. 40A-40F). The current flow was modelled using the Electric Current (EC) physics from the AC/DC module. The governing equations of the stationary study were used as follows:

∇·J=Q_{j,v} (9)

J=σE+Jₑ (10)

E=−∇V (11)

where J is the current density, Q_{j,v} is the volumetric source current, E is electrical field, Jₑ is the externally generated current density, V is electrical potential. The trachea tissue model was represented as a hollow cylinder (inner diameter: 3.5% mm; thickness: 500% μm; length: 15% mm). Four electrode probes (material: tungsten; diameter: 500% μm) were arranged in-line and placed on the surface of the trachea tissue. The electrical conductivity of trachea tissue and the tungsten electrodes was obtained from COMSOL library and the literature. In order to ensure that the stationary problem was well-defined, the conductivity of the air was considered very low but nonzero (conductivity value: 10⁻⁹ S/m). Moreover, as the model was solved under the quasi-static assumption, it was insensitive to changes in relative permittivity; therefore, a default value of 1 was assigned to all the materials. An extremely fine mesh (shape: free triangular) was created for the simulation, and the solution was obtained for the stationary study with default COMSOL Multiphysics 6.0 settings. Direct electrical current (DC, level: 100% μA) was injected into the tissue through a current-carrying probe (i.e., current terminal), while the other outer probe served as the ground. Current density normal (J, expression: ec.normJ; unit: A/m²) which describes the strength of the electric current flow was obtained for the tissue volume and the cut planes.

All data were presented as mean±standard deviation (SD). All experiments were performed at least three times (n>3, n: number of samples) to ensure reproducibility and to evaluate statistical significance between experiment groups. One-way analysis of variance (One-way ANOVA) followed by Tukey's honest significant post hoc test was used for comparison between different groups, with p<0.05 considered statistically significant.

For a linear arrangement of electrode probes with unequal spacing on a 3D sample (FIG. 32A-32B), the voltage drop between two voltage-sensing probes (ΔV=V2−V3) can be calculated using the following equation:

$$V = {V}_{2} - {V}_{3} = \frac{I ρ}{2 π} [ ( \frac{1}{{s}_{1}} - \frac{1}{{s}_{2}} ) - ( \frac{1}{{s}_{3}} - \frac{1}{{s}_{4}} ) ] \qquad ( 1 )$$

where I is the current injected into the sample through the current-carrying probes, p is the resistivity of the sample, and s₁ (i=1, 2, 3, 4) is the distance between the probes. To investigate the effect of probe spacing, the distance between the voltage-sensing probes (a) was kept constant at 3 mm, and the distance between the voltage-sensing and current-carrying robes (b) was varied (short: 2 mm, medium: 4 mm, long: 6 mm):

| | s₁ (mm) | s₂ (mm) | s₃ (mm) | s₄ (mm) |
|---|---|---|---|---|
| Short | 2 | 5 | 5 | 2 |
| Medium | 4 | 7 | 7 | 4 |
| Long | 6 | 9 | 9 | 6 |

The voltage drop between the inner probes (11 and sample resistance R_{3D}=VII were calculated as follows:

| | Short | Medium | Long |
|---|---|---|---|
| V | 0.3Iρ/π | 0.1Iρ/π | 0.05Iρ/π |
| R_{3D} | 0.3ρ/π | 0.1ρ/π | 0.05ρ/π |

This indicates that increasing the spacing b reduces the resistance between the voltage-sensing probes.

Further details relating to the present invention can be found in the paper entitled “Bioimpedance Measurements of Fibrotic and Acutely Injured Lung Tissues” by Mir et al., the entirety of which is incorporated herein by reference and made a part of the present application for all purposes.

It will be understood that the embodiments described herein are merely exemplary and that a person skilled in the art may make many variations and modifications without departing from the spirit and scope of the invention. All such variations and modifications are intended to be included within the scope of the invention.

What is claimed is:

1. A method of assessing tissue function or conditions, comprising the steps of:
placing a probe in contact with a tissue of interest, said probe comprising at least one electrode configured to deliver electrical current to the tissue of interest and at least another electrode configured to take potential measurements of the tissue of interest;
delivering electrical current to the tissue of interest via said at least one electrode of said probe;
taking potential measurements of the tissue of interest using said at least another electrode of said probe;
calculating impedance measurements in response to said potential measurements; and
assessing tissue function or conditions based on said impedance measurements.

2. The method of claim 1, wherein said at least one electrode and/or said at least another electrode comprise tungsten.

3. The method of claim 1, further comprising the step of placing said at least one electrode and/or said at least another electrode on said tissue using a micropositioner device.

4. The method of claim 1, further comprising the step of monitoring the position of said at least one electrode and said at least another electrode relative to the tissue of interest using a video bronchoscopy device.

5. The method of claim 1, wherein said assessing step comprises the step of monitoring the spatial arrangement of genes or gene-editing machinery in proximity to the tissue of interest.

6. The method of claim 5, wherein said delivering step involves delivering high frequency current to determine the spatial arrangement of genes and/or gene-editing machinery internalized within cells.

7. The method of claim 5, wherein said delivering step involves delivering low frequency current to determine the spatial arrangement of genes and/or gene-editing machinery external to cells.

8. The method of claim 1, wherein said tissue is lung tissue, and further comprises the steps of depositing genes or gene editing machinery on the luminal surface of the lung tissue and electroporating the luminal surface to internalize said genes or gene-editing machinery into cells.

9. The method of claim 1, wherein the tissue of interest is a donor organ.

10. The method of claim 9, further comprising the step of using said impedance measurements to localize injured or diseased tissue or defects in the tissue of interest.

11. The method of claim 1, wherein the tissue of interest is lung tissue, and wherein said assessing step involves assessment of a mucus layer in the respiratory tract.

12. The method of claim 1, further comprising the step of diagnosing one or more airway diseases using said impedance measurements.

13. The method of claim 1, wherein said at least one electrode and/or said at least another electrode comprise functionalized surfaces.

14. The method of claim 13, further comprising the step of recognizing and binding said functionalized surfaces to specific target molecules, proteins, or pathogens.

15. The method of claim 14, further comprising the steps of measuring change in electrical properties of said at least one electrode and/or said at least another electrode following said recognizing and binding step.

16. The method of claim 1, wherein said delivering and taking steps are performed via a frequency sweep of said electrical current.

17. The method of claim 16, wherein said frequency sweep is conducted in a frequency range from 100 Hz to 300 kHz.

18. The method of claim 16, wherein said frequency sweep is conducted in a frequency range from 100 Hz to 135 kHz.

19. The method of claim 16, wherein said frequency sweep is conducted in a frequency range from 30 Hz to 300 kHz.

20. The method of claim 16, wherein said electrical current is direct current.

21. The method of claim 1, wherein said delivering step includes the step of varying measurement parameters for variable penetration of electrical current into the tissue of interest, thereby varying the depth of penetration of said electrical current and allowing for said impedance measurements at different depths.

22. The method of claim 21, wherein said measurement parameters include frequency of said electrical current, spatial arrangement of said at least one electrode and/or said at least another electrode, and size of said at least one electrode and/or said at least another electrode.

23. Apparatus for evaluating the function or conditions of a tissue of interest, comprising:
a probe with a plurality of electrodes adapted to be placed in direct contact with a tissue of interest, including at least one electrode adapted to deliver electrical current to the tissue of interest and at least another electrode adapted to make potential measurements of the tissue of interest; and
an impedance analyzer adapted to receive said potential measurements from said probe, wherein said impedance analyzer is adapted to evaluate tissue properties or conditions based on said potential measurements.

24. The apparatus of claim 23, further comprising a micropositioner adapted to place said at least one electrode and/or said at least another electrode on the tissue of interest.

25. The apparatus of claim 23, wherein said apparatus is integrated in a video bronchoscopy device.

26. The apparatus of claim 23, wherein said apparatus is integrated as part of a robotic surgery device.

27. The apparatus of claim 23, wherein said impedance analyzer is adapted to determine the presence of defects or diseased tissue in a donor organ.

28. The apparatus of claim 23, wherein said impedance analyzer is adapted to determine the presence or condition of a mucus layer in the respiratory tract.

29. The apparatus of claim 23, wherein said at least one electrode and/or said at least another electrode comprise surfaces functionalized with molecules, proteins, aptamers or antibodies, such that said at least one electrode and/or said at least another electrode can recognize and bind to specific target molecules, proteins, or pathogens.

30. The apparatus of claim 29, wherein said specific target molecules are pathogens associated with airway injury or disease.
